(19) United States
(12) Patent Application Publication
(10) Pub. No.: US 2025/0041325 A1
(43) Pub. Date: Feb. 6, 2025

(54) LIPID-BASED COMPOSITIONS AND METHODS THEREOF

(71) Applicants: The Brigham and Women's Hospital, Inc., Boston, MA (US); The General Hospital Corporation, Boston, MA (US)

(72) Inventors: Yuling Xiao, Brookline, MA (US); Gabriel D Duda, Belmont, MA (US); Jinjun Shi, Newton, MA (US)

(21) Appl. No.: 18/716,970

(22) PCT Filed: Dec. 7, 2022
(86) PCT No.: PCT/US2022/052158

§ 371 Date: Jun. 6, 2024

Related U.S. Application Data
(60) Provisional application No. 63/286,786, filed on Dec. 7, 2021.

(51) Int. Cl. A61K 31/7105; A61K 47/54; A61N 5/10; A61P 35/04; C07K 16/18

(52) U.S. Cl. CPC A61K 31/7105; A61K 47/543; A61N 5/10; A61P 35/04; C07K 16/18

(57) ABSTRACT

The present disclosure relates to compositions having a lipid-like compound of Gₘ-Cₙ, as well as methods of making and using such compositions. Also described herein are methods of treating cancer using such compounds with a combination therapy.

### CROSS-REFERENCE TO RELATED APPLICATION

This application claims the benefit of U.S. Provisional Patent Application No. 63/286,786, filed on Dec. 7, 2021, which is incorporated by reference herein in its entirety.

### STATEMENT OF GOVERNMENT INTEREST

This invention was made with Government support under Contract No. W81XWH1910482 awarded by the U.S. Department of Defense. The Government has certain rights in the invention.

### FIELD

The present disclosure relates to compositions having a lipid-like compound of Gₘ-Cₙ, as well as methods of making and using such compositions. Also described herein are methods of treating cancer using such compounds in combination with other therapies.

### BACKGROUND

Targeted delivery of cargo to cells remains a challenge. Particular difficulties remain for therapies to target cancer cells, such as those characterized by the loss or mutation of tumor suppressor genes.

### SUMMARY

The present disclosure relates to compositions including a lipid-like compound of Gₘ-Cₙ, as well as methods of using compositions for treatment (e.g., treatment of cancer with other combination therapies). In particular embodiments, such compounds can be used to form particles (e.g., nanoparticles) that can deliver cargo (e.g., any described herein, such as mRNA). Such particles can have any useful form, such as lipid particles, solid lipid particles, liposomes, micelles, and the like.

Accordingly, in a first aspect, the present disclosure describes the use of particles for treating cancer. In addition to providing a therapeutic agent by way of such particles, treatment can be combined with other therapies, such as, e.g., immunotherapy, anti-angiogenesis therapy, or radiotherapy. As described herein, such particles can be used to deliver p53-encoding mRNA to p53-deficient cancer cells. Thus, in certain embodiments, methods can include administering a therapeutically effective amount of an mRNA encoding tumor suppressor protein p53 to a subject in need thereof. Such methods can be used in conjunction with other therapies, in which methods can then include administering a therapeutically effective amount of p53-encoding mRNA with a combination therapy to a subject in need thereof.

In some embodiments, the present disclosure encompasses a method of treating a cancer, the method including administering a therapeutically effective amount of an mRNA encoding tumor suppressor protein p53 with a combination therapy to a subject in need thereof.

In some embodiments, the combination therapy includes immunotherapy, anti-angiogenesis therapy, radiotherapy, or a combination thereof.

In some embodiments, the immunotherapy includes administering a therapeutically effective amount of at least one immune checkpoint inhibitor to the subject. Non-limiting immune checkpoint inhibitors include an anti-PD1 antibody, an anti-PD-L1 antibody, an anti-CTLA-4 antibody, an anti-TIM3 antibody, an anti-LAG3 antibody, an anti-CD137 antibody, an anti-CD40 antibody, as well as any others described herein.

In some embodiments, the anti-angiogenesis therapy includes administering a therapeutically effective amount of at least one angiogenesis inhibitor to the subject. Non-limiting angiogenesis inhibitors include an anti-VEGF antibody, an anti-VEGF receptor (VEGFR) antibody, a VEGF receptor kinase inhibitor, an anti-FGF antibody, an anti-FGF receptor (FGFR) antibody, an FGF receptor kinase inhibitor, an anti-PDGF antibody, an anti-PDGF receptor (PDGFR) antibody, a PDGF receptor kinase inhibitor, an anti-EGF antibody, an anti-EGF receptor (EGFR) antibody, an EGF receptor kinase inhibitor, as well as any others described herein.

In some embodiments, the radiotherapy includes administering a therapeutically effective amount of irradiation to the subject. In particular embodiments, the irradiation includes x-rays, gamma rays, electron beam radiation, proton beam radiation, or ionizing particles.

In some embodiments, the p53-encoding mRNA is within a delivery vehicle capable of providing release of the p53-encoding mRNA in a cancer cell. In particular embodiments, the delivery vehicle is a particle including the p53-encoding mRNA and a complexing agent within a core. In other embodiments, the particle further includes an outer layer including at least one amphiphilic material disposed around the core. In some embodiments, the amphiphilic material is selected from lecithin, a phospholipid (e.g., phosphatidic acid, phosphatidylcholine, phosphatidylethanolamine, phosphatidylglycerol, phosphatidylserine, phosphatidylinositol, lysophosphatidyl, cardiolipin, or β-acyl-y-alkyl phospholipid), and a pegylated lipid (e.g., ceramide-polyethylene glycol (PEG), 1,2-distearoyl-sn-glycero-3-phosphoethanolamine (DSPE)-terminated PEG, and any others described herein). In yet other embodiments, the complexing agent is selected from a cationic lipid (e.g., any described herein), an ionizable lipid-like compound (e.g., Gₘ-Cₙ, wherein m≥0 and n<20, as well as any described herein), or an ionizable lipid (e.g., DLin-MC3-DMA, SM-102, ALC-0315, as well as any described herein).

In some non-limiting embodiments, the core further includes a water-insoluble polymer. Non-limiting water-insoluble polymers include a polyester selected from a group consisting of poly(lactic-co-glycolic acid) (PLGA), poly(lactic acid) (PLA), and poly(glycolic acid) (PGA); or a copolymer of polyethylene glycol (PEG) and a polyester selected from PLGA, PLA, and PGA.

In a second aspect, the present disclosure describes the use of particular cargos with targeting ligands to target certain cells. For example and without limitation, the cargo can encode a tumor suppressor, and the targeting ligand can be configured to bind to receptors or other moieties disposed on a target cell lacking the tumor suppressor. In this way, delivery of the tumor suppressor can restore proper regulation to that cell.

In some embodiments, the composition includes: a core and an outer layer surrounding the core. In particular embodiments, the core includes: a tumor suppressor-encoding mRNA (e.g., a p53-encoding mRNA, a PTEN-encoding mRNA, or any others described herein); an ionizable lipid-like compound of Gₘ-Cₙ, wherein m≥0 and n<20; and an optional water-insoluble polymer (e.g., PLGA or any others described herein). In some embodiments, the outer layer includes: a targeting ligand for a chemokine receptor (e.g., CXCR4, GPC3, or any others described herein); and a pegylated lipid (e.g., a lipid-PEG or any others described herein).

In some embodiments, the targeting ligand includes: a targeting moiety configured to bind to the chemokine receptor, a pegylated lipid configured to form a portion of the outer layer, and a linker disposed between the targeting moiety and the pegylated lipid.

In some embodiments, the tumor suppressor-encoding mRNA includes an mRNA encoding a p53 protein, and/or the targeting ligand includes a CXCR4-targeting ligand. In particular embodiments, the CXCR4-targeting ligand includes a CXCR4-targeting moiety bound to a lipid and an optional linker (e.g., a PEG linker) disposed between the CXCR4-targeting moiety and the lipid. In other embodiments, the CXCR4-targeting moiety includes a sequence having at least 80% sequence identity to KGVSLSYRCRYSLSVGK (SEQ ID NO: 1) or any one of SEQ ID NOs: 20-39 (e.g., as described herein), or a fragment thereof.

In some embodiments, the tumor suppressor-encoding mRNA includes an mRNA encoding a p53 protein, and/or the targeting ligand includes a GPC3-targeting ligand.

In particular embodiments, the GPC3-targeting ligand includes a GPC3-targeting moiety bound to a lipid and an optional linker (e.g., a PEG linker) disposed between the GPC3-targeting moiety and the lipid. In other embodiments, the GPC3-targeting moiety includes a sequence having at least 80% sequence identity to THVSPNQGGLPS (SEQ ID NO: 7), RLNVGGTYFLTTRQ (SEQ ID NO: 8), SNDRPPNILQKR (SEQ ID NO: 9), or a fragment thereof.

In some embodiments, a density of the targeting ligand (e.g., CXCR4-targeting ligand, GPC3-targeting ligand, and the like) is between about 3% to about 10% (e.g., as determined within the outer layer).

In some embodiments, the outer layer further includes a pegylated lipid (e.g., DSPE-PEG), a lipid, or a combination thereof.

In some embodiments, the ionizable lipid-like compound of Gₘ-Cₙ, and m≥0 and n<20; m is an integer from 0 to 5, and n is an integer from 6 to 18; and the like. In other embodiments, the ionizable lipid-like compound includes G₀-C₈, G₀-C₁₀, G₀-C₁₂, G₁-C₈, G₁-C₁₀, G₁-C₁₂, G₁-C₁₄, G₂-C₈, G₂-C₁₀, G₂-C₁₂, G₂-C₁₄, G₃-C₈, G₃-C₁₀, G₃-C₁₂, G₃-C₁₄, G₄-C₈, G₄-C₁₀, G₄-C₁₂, or G₄-C₁₄. In yet other embodiments, the ionizable lipid-like compound is not G₀-C₁₄.

In some embodiments, a weight ratio of the ionizable lipid-like compound to the mRNA is from about 1:1 to about 40:1 (wt:wt).

In some embodiments, the core and the outer layer forms a nanoparticle. In other embodiments, the nanoparticle has an average size from about 80 nm to about 150 nm.

In a third aspect, the present disclosure describes a lipid-like compound of Gₘ-Cₙ to complex a cargo within a core of a delivery vehicle. For example and without limitation, structure-activity relationships are provided for Gₘ-Cₙ compounds having varying combinations of m and n values, which can impart effective stabilization and/or delivery of the cargo. Accordingly, described herein are Gₘ-Cₙ compounds for use with cargos.

In some embodiments, the core includes: a nucleic acid; a lipid-like compound of Gₘ-Cₙ, wherein (i) m=0 and n<14; or (ii) m>0 and n<20; and an optional water-insoluble polymer (e.g., PLGA). In some embodiments, m=0 and n<14. In some embodiments, m>0 and n<20. In some embodiments, m is 0; and n is an integer from about 6 to about 12. In some embodiments, m is 1; and n is an integer from about 6 to about 12. In some embodiments, m is 2, 3, or 4; and n is an integer from about 6 to about 14. In some embodiments, m is 0, 1, 2, or 3; and n is about 8. In some embodiments, m is 3 or 4; and n is about 14.

In some embodiments, the composition includes an outer layer surrounding the core, wherein the outer layer includes: a lipid or a pegylated lipid (e.g., DSPE-PEG); and an optional targeting ligand. In other embodiments, the outer layer includes the lipid and the pegylated lipid.

In some embodiments, the composition includes a targeting ligand. Non-limiting target ligands include a protein, a peptide, an aptamer, a nucleic acid, a monosaccharide, a polysaccharide, a carbohydrate, a vitamin, or a small molecule, as well as any described herein.

In a fourth aspect, the present disclosure encompasses a formulation including a therapeutically effective amount of a composition (e.g., any described herein) and a pharmaceutically acceptable excipient (e.g., any described herein). In some embodiments, the formulation is formulated for injection, implantation, and the like.

In a fifth aspect, the present disclosure encompasses a method of treating cancer, the method including: administering a therapeutically effective amount of a composition (e.g., any described herein) or a formulation (e.g., any described herein) to a subject in need thereof.

In some embodiments, the cancer is p53-deficient cancer, a p53-deficient primary liver cancer (e.g., hepatocellular carcinoma or cholangiocarcinoma), or liver metastases from a p53-deficient cancer (colon, lung, pancreatic, etc.). In other embodiments, the p53-deficient cancer is primary colon cancer, primary lung cancer, primary liver cancer, primary hepatocellular carcinoma, primary cholangiocarcinoma, primary pancreatic cancer, metastatic colon cancer, metastatic lung cancer, metastatic liver cancer, or metastatic pancreatic cancer.

In some embodiments, the method further includes: administering a therapeutically effective amount of at least one immune checkpoint inhibitor at a timepoint before, after, or during said administering the composition. In other embodiments, the at least one immune checkpoint inhibitor is administered at a timepoint before, after, or during administering of an angiogenesis inhibitor. In yet other embodiments, the at least one immune checkpoint inhibitor is administered at a timepoint before, after, or during administering of irradiation. In particular embodiments, the at least one immune checkpoint inhibitor includes an anti-PD-1 (aPD1) antibody, an anti-PD-L1 (aPD-L1) antibody, an anti-CTLA-4 antibody, an anti-TIM3 antibody, an anti-LAG3 antibody, an anti-CD137 antibody, or an anti-CD40 antibody.

In some embodiments, the method further includes: administering a therapeutically effective amount of at least one angiogenesis inhibitor at a timepoint before, after, or during said administering the composition. In other embodiments, the at least one angiogenesis inhibitor is administered at a timepoint before, after, or during said administering an immune checkpoint inhibitor. In yet other embodiments, the at least one angiogenesis inhibitor is administered at a timepoint before, after, or during said administering irradiation. In some embodiments, the at least one angiogenesis inhibitor includes an anti-VEGF antibody, an anti-VEGF receptor antibody, a VEGF receptor kinase inhibitor, an anti-FGF antibody, an anti-FGF receptor antibody, an FGF receptor kinase inhibitor, an anti-PDGF antibody, an anti-PDGF receptor antibody, a PDGF receptor kinase inhibitor, an anti-EGF antibody, an anti-EGF receptor antibody, or an EGF receptor kinase inhibitor, as well as any others described herein.

In some embodiments, the method further includes: administering a therapeutically effective amount of irradiation at a timepoint before, after, or during said administering the composition. In other embodiments, irradiation is administered at a timepoint before, after, or during said administering an immune checkpoint inhibitor. In other embodiments, irradiation is administered at a timepoint before, after, or during said administering an angiogenesis inhibitor.

In a sixth aspect, the present disclosure encompasses a method of modulating an interaction between a tumor and an immune cell, the method including: administering a therapeutically effective amount of a composition (e.g., any disclosed herein) or a formulation (e.g., any disclosed herein) to a subject in need thereof. In some embodiments, the immune cell includes an NK cell, a T cell, and/or a tumor-associated macrophage (TAM).

In some embodiments, the method further includes: administering a therapeutically effective amount of at least one immune checkpoint inhibitor at a timepoint before, after, or during said administering the composition. In some embodiments, the at least one immune checkpoint inhibitor includes an anti-PD-1 (aPDI) antibody, an anti-PD-L1 (aPD-L1) antibody, an anti-CTLA-4 antibody, an anti-TIM3 antibody, an anti-LAG3 antibody, an anti-CD137 antibody, or an anti-CD40 antibody.

In some embodiments, the method further includes: administering a therapeutically effective amount of at least one VEGF inhibitor (e.g., an anti-VEGF antibody, an anti-VEGFR2 antibody, an anti-VEGFR kinase inhibitor, or any described herein) at a timepoint before, after, or during said administering the composition.

In some embodiments, the method further includes: administering a therapeutically effective amount of irradiation at a timepoint before, after, or during said administering the composition.

In a seventh aspect, the present disclosure encompasses a method of making a composition, the method including: complexing an mRNA with a lipid-like compound of Gₘ-Cₙ in an acidic environment, wherein m≥0 and n<20, in the presence of an optional water-insoluble polymer, thereby forming a core; and surrounding the core with an outer layer including a lipid, a pegylated lipid, or a target ligand, thereby providing the composition.

In some embodiments, the acidic environment includes a pH from about 4 to about 2. In other embodiments, the acidic environment includes an acidic buffered solution.

In some embodiments, said forming includes the water-insoluble polymer in an organic solvent.

In some embodiments, said surrounding includes stirring at a rate of about 500 rpm to about 1500 rpm. In other embodiments, the outer layer includes the pegylated lipid and the target ligand (e.g., a CXCR4-targeting ligand). In yet other embodiments, said surrounding includes providing the target ligand.

In some embodiments, the method provides any composition described herein.

In any embodiment herein, the cancer is associated with loss of p53 expression or activity.

In any embodiment herein, the cancer is selected from liver cancer, lung cancer, prostate cancer, breast cancer, glioblastoma, melanoma, pancreatic cancer, colorectal cancer, and leukemia, as well as any others described herein.

In any embodiment herein, the composition, the delivery vehicle, or the particle can include at least one amphiphilic material. Non-limiting amphiphilic materials includes lecithin, a phospholipid, a pegylated lipid, as well as others described herein.

In any embodiment herein, the composition, the delivery vehicle, or the particle can include at least one lipid. Non-limiting lipids include a neutral lipid, a cationic lipid, an anionic lipid, an ionizable lipid (e.g., a cationic ionizable lipid), a pegylated lipid, as well as any others described herein.

In any embodiment herein, the composition, the delivery vehicle, or the particle can include at least one pegylated lipid (e.g., a lipid-PEG), e.g., any described herein.

In any embodiment herein, the composition, the delivery vehicle, or the particle can include at least one ionizable lipid-like compound. Non-limiting ionizable lipid-like compounds include Gₘ-Cₙ, wherein m≥0 and n<20; or wherein m=0 and n<14; or wherein m>0 and n<20; or wherein m is 0 and n is an integer from about 6 to about 12; or wherein m is 1 and n is an integer from about 6 to about 12; or wherein m is 2, 3, or 4 and n is an integer from about 6 to about 14; or wherein m is 0, 1, 2, or 3, and n is about 8; or wherein m is 3 or 4 and n is about 14; or wherein m is an integer from 0 to 5 and n is an integer from 6 to 18. In yet other embodiments, the compound is or includes G₀-C₈, G₀-C₁₀, Go-C₁₂, G₁-C₈, G₁-C₁₀, G₁-C₁₂, G₁-C₁₄, G₂-C₈, G₂-C₁₀, G₂-C₁₂, G₂-C₁₄, G₃-C₈, G₃-C₁₀, G₃-C₁₂, G₃-C₁₄, G₄-C₈, G₄-C₁₀, G₄-C₁₂, or G₄-C₁₄. In other embodiments, the compound is not G₀-C₁₄.

In any embodiment herein, the composition, the delivery vehicle, or the particle can include at least one water-insoluble polymer. In particular embodiments, the composition, the delivery vehicle, or the particle includes a hybrid polymer-lipid particle.

In any embodiment herein, the composition, the delivery vehicle, or the particle does not include a water-insoluble polymer. In particular embodiments, the composition, the delivery vehicle, or the particle includes a lipid particle.

In any embodiment herein, the composition, the delivery vehicle, or the particle can include at least one complexing agent. Non-limiting complexing agents include a cationic lipid (e.g., any described herein), an ionizable lipid-like compound (e.g., any described herein), or an ionizable lipid (e.g., any described herein).

In any embodiment herein, the composition, the delivery vehicle, or the particle can include at least one tumor suppressor-encoding mRNA. Non-limiting tumor suppressor-encoding mRNAs include, a p53-encoding mRNA, a PTEN-encoding mRNA, as well as any described herein.

In any embodiment herein, the composition, the delivery vehicle, or the particle can include at least one targeting ligand. In some embodiments, the targeting ligand includes a targeting moiety. In other embodiments, the targeting ligand includes a pegylated lipid and a linker disposed between the targeting moiety and the pegylated lipid (e.g., any described herein). Non-limiting targeting moieties include CXCR4, GPC3, or any described herein.

Additional details follow.

### Definitions

By “aliphatic” is meant a hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well. Yet other aliphatic groups can include C₁₋₁₀, C₁₋₁₂, C₁₋₁₃, C₁₋₁₄, C₁₋₁₅, C₁₋₁₆, C₁₋₁₇, C₁₋₁₈, C₂₋₁₀, C₂₋₁₂, C₂₋₁₃, C₂₋₁₄, C₂₋₁₅, C₂₋₁₆, C₂₋₁₇, C₂₋₁₅, C₃₋₁₀, C₃₋₁₂, C₃₋₁₃, C₃₋₁₄, C₃₋₁₅, C₃₋₁₆, C₃₋₁₇, C₃₋₁₅, C₄₋₁₀, C₄₋₁₂, C₄₋₁₃, C₄₋₁₄, C₄₋₁₅, C₄₋₁₆, C₄₋₁₇, C₄₋₁₅, C₅₋₁₀, C₅₋₁₂, C₅₋₁₃, C₅₋₁₄, C₅₋₁₅, C₅₋₁₆, C₅₋₁₇, C₅₋₁₅, C₆₋₁₀, C₆₋₁₂, C₆₋₁₃, C₆₋₁₄, C₆₋₁₅, C₆₋₁₆, C₆₋₁₇, C₆₋₁₅, C₇₋₁₀, C₇₋₁₂, C₇₋₁₃, C₇₋₁₄, C₇₋₁₅, C₇₋₁₆, C₇₋₁₇, C₇₋₁₅, C₈₋₁₀, C₈₋₁₂, C₈₋₁₃, C₈₋₁₄, C₈₋₁₅, C₈₋₁₆, C₈₋₁₇, or C₈₋₁₈ aliphatic groups. The aliphatic group can also be substituted or unsubstituted. For example, the aliphatic group can be substituted with one or more substitution groups, as described herein for alkyl.

By “alkyl” and the prefix “alk” is meant a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C₃₋₂₄ cycloalkyl) or acyclic. The alkyl group can be branched or unbranched. The alkyl group can also be substituted or unsubstituted. For example, the alkyl group can be substituted with one, two, three or, in the case of alkyl groups of two carbons or more, four substituents independently selected from the group consisting of. (1) C₁₋₆ alkoxy (e.g., —OAk, in which Ak is an alkyl group, as defined herein); (2) C₁₋₆ alkylsulfinyl (e.g., —S(O)Ak, in which Ak is an alkyl group, as defined herein); (3) C₁₋₆ alkylsulfonyl (e.g., —SO₂Ak, in which Ak is an alkyl group, as defined herein); (4) amino (e.g., —NR^{N1}R^{N2} where each of R^{N1} and R^{N2} is, independently, H or optionally substituted alkyl, or R^{N1} and R^{N2}, taken together with the nitrogen atom to which each are attached, form a heterocyclyl group); (5) aryl; (6) arylalkoxy (e.g., —OA^{L}Ar, in which A^{L} is an alkylene group and Ar is an aryl group, as defined herein); (7) aryloyl (e.g., —C(O)Ar, in which Ar is an aryl group, as defined herein); (8) azido (e.g., an —N₃ group); (9) cyano (e.g., a —CN group); (10) carboxyaldehyde (e.g., a —C(O)H group); (11) C₃₋₈ cycloalkyl; (12) halo; (13) heterocyclyl (e.g., a 5-, 6- or 7-membered ring, unless otherwise specified, containing one, two, three, or four non-carbon heteroatoms (e.g., independently selected from the group consisting of nitrogen, oxygen, phosphorous, sulfur, or halo)); (14) heterocyclyloxy (e.g., —OHet, in which Het is a heterocyclyl group); (15) heterocyclyloyl (e.g., —C(O)Het, in which Het is a heterocyclyl group); (16) hydroxyl (e.g., a —OH group); (17)N-protected amino; (18) nitro (e.g., an —NO₂ group); (19) oxo (e.g., an═O group); (20) C₃₋₈ spirocyclyl (e.g., an alkylene diradical, both ends of which are bonded to the same carbon atom of the parent group to form a spirocyclyl group); (21) C₁₋₆ thioalkoxy (e.g., —SAk, in which Ak is an alkyl group, as defined herein); (22) thiol (e.g., an —SH group); (23) —CO₂R^{A}, where R^{A}is selected from the group consisting of (a) hydrogen, (b) C₁₋₆ alkyl, (c) C₄₋₁₈ aryl, and (d) C₁₋₆ alk-C₄₋₁₈ aryl; (24) —C(O)NR_{B}R^{C}, where each of R^{B} and R^{C} is, independently, selected from the group consisting of (a) hydrogen, (b) C₁₋₆ alkyl, (c) C₄₋₁₈ aryl, and (d) C₁₋₆ alk-C₄₋₁₈ aryl; (25) —SO₂R^{D}, where R^{D} is selected from the group consisting of (a) C₁₋₆ alkyl, (b) C₄₋₁₈ aryl, and (c) C₁₋₆ alk-C₄₋₁₈ aryl; (26) —SO₂NR^{E}R^{F}, where each of R^{E} and R^{F} is, independently, selected from the group consisting of (a) hydrogen, (b) C₁₋₆ alkyl, (c) C₄₋₁₈ aryl, and (d) C₁₋₆ alk-C₄₋₁₈ aryl; and (27) —NR^{G}R^{H}, where each of R^{G} and R^{H} is, independently, selected from the group consisting of (a) hydrogen, (b) an N-protecting group, (c) C₁₋₆ alkyl, (d) C₂₋₆ alkenyl, (e) C₂₋₆ alkynyl, (f) C₄₋₁₈ aryl, (g) C₁₋₆ alk-C₄₋₁₈ aryl, (h) C₃₋₈ cycloalkyl, and (i) C₁₋₆ alk-C₃₋₈ cycloalkyl, wherein in one embodiment no two groups are bound to the nitrogen atom through a carbonyl group or a sulfonyl group. The alkyl group can be a primary, secondary, or tertiary alkyl group substituted with one or more substituents (e.g., one or more halo or alkoxy). In some embodiments, the unsubstituted alkyl group is a C₁₋₃, C₁₋₆, C₁₋₁₂, C₁₋₁₆, C₁₋₁₈, C₁₋₂₀, or C₁₋₂₄ alkyl group.

By “alkylene” is meant a multivalent (e.g., bivalent, trivalent, tetravalent, etc.) form of an alkyl group, as described herein. Exemplary alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₆, C₂₋₂₀, or C₂₋₂₄ alkylene group. The alkylene group can be branched or unbranched. The alkylene group can also be substituted or unsubstituted. For example, the alkylene group can be substituted with one or more substitution groups, as described herein for alkyl.

By “alkenyl” is meant an unsaturated monovalent hydrocarbon having at least two carbon atom to 50 carbon atoms (C_{2-═}), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z). An exemplary alkenyl includes an optionally substituted C₂₋₂₄ alkyl group having one or more double bonds. The alkenyl group can be monovalent or multivalent (e.g., bivalent) by removing one or more hydrogens to form appropriate attachment to the parent molecular group or appropriate attachment between the parent molecular group and another substitution. The alkenyl group can also be substituted or unsubstituted. For example, the alkenyl group can be substituted with one or more substitution groups, as described herein for alkyl. In some embodiments, the unsubstituted alkenyl group is a C₂₋₃, C₂₋₆, C₂₋₁₂, C₂₋₁₆, C₂₋₁₆, C₂₋₂₀, or C₂₋₂₄ alkenyl group.

By “alkynyl” is meant an unsaturated monovalent hydrocarbon having at least two carbon atom to 50 carbon atoms (C_{2-═}), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl). An exemplary alkynyl includes an optionally substituted C₂₋₂₄ alkyl group having one or more triple bonds. The alkynyl group can be cyclic or acyclic and is exemplified by ethynyl, 1-propynyl, and the like. The alkynyl group can be monovalent or multivalent (e.g., bivalent) by removing one or more hydrogens to form appropriate attachment to the parent molecular group or appropriate attachment between the parent molecular group and another substitution. The alkynyl group can also be substituted or unsubstituted. For example, the alkynyl group can be substituted with one or more substitution groups, as described herein for alkyl. In some embodiments, the unsubstituted alkynyl group is a C₂₋₃, C₂₋₆, C₂₋₁₂, C₂₋₁₆, C₂₋₁₆, C₂₋₂₀, or C₂₋₂₄ alkynyl group.

By “amide” or “amido” is meant a —C(═O)NR^{N1}— moiety, a —NRN¹C(═O)— moiety, or a compound including any of these moieties, in which R^{N1} is H or optionally substituted aliphatic (e.g., as described herein, including optionally substituted C₁₋₁₂ aliphatic or C₁₋₁₂ alkyl).

By “amino” or “amine” is meant a —NR^{N1}R^{N2} moiety, a —NR^{N1}— moiety, or a compound having any of these moieties, in which each of R^{N1} and R^{N2}, independently, can be H or aliphatic (e.g., as described herein, including optionally substituted C₁₋₁₂ aliphatic or C₁₋₁₂ alkyl).

By “heteroalkyl” is meant an alkyl group, as defined herein, containing one, two, three, or four non-carbon heteroatoms (e.g., independently selected from the group consisting of nitrogen, oxygen, phosphorous, sulfur, or halo). In turn, such heteroatoms can provide other functional groups. Non-limiting heteroatom-containing functional groups include —NR^{N1}—, —C(O)NR^{N1}—, —NR^{N1}C(O)—, —C(O)NR^{N1}C(O)—, —C(O)—, —O—, ═O, ═NR^{N1}, —C(NR^{N1})—, —SO₂—, —S—, —P(O)OR^{P1}—, —P(O)R^{P1}—, and the like, in which each R^{N1} and R^{P1} is, independently, H or optionally substituted aliphatic (e.g., as described herein, including optionally substituted C₁₋₁₂ aliphatic or C₁₋₁₂ alkyl).

By “heteroalkylene” is meant a divalent form of an alkyl group, as defined herein, containing one, two, three, or four non-carbon heteroatoms (e.g., independently selected from the group consisting of nitrogen, oxygen, phosphorous, sulfur, or halo). In other embodiments, the heteroalkylene is a divalent form of any heteroalkyl group described herein.

By “linker” is meant any useful multivalent (e.g., bivalent) component useful for joining to different portions or segments. Exemplary linkers include a bond, a nucleic acid sequence, a chemical linker, etc. The linker may include a covalent linker or a non-covalent linker. In some embodiments: the linker may comprise a flexible arm, e.g., 2, 3, 4, 5, 6, 7, 8, 9, 10, 11, 12, 13, 14, or 15 carbon atoms. Exemplary linkers include BS3 ([bis(sulfo-succinimidyl)suberate]; BS3 is a homobifunctional N-hydroxysuccinimide ester that targets accessible primary amines), NHS/EDC (N-hydroxysuccinimide and N-ethyl-N-(dimethylaminopropyl)carbodiimide; NHS/EDC allows for the conjugation of primary amine groups with carboxyl groups), sulfo-EMCS ([N-ε-maleimidocaproic acid]hydrazide; sulfo-EMCS are heterobifunctional reactive groups (maleimide and NHS-ester) that are reactive toward sulfhydryl and amino groups), hydrazide (most proteins contain exposed carbohydrates and hydrazide is a useful reagent for linking carboxyl groups to primary amines), and SATA (N-succinimidyl-S-acetylthioacetate; SATA is reactive towards amines and adds protected sulfhydryls groups). Examples of other suitable linkers are succinic acid, Lys, Glu, Asp, a dipeptide such as Gly-Lys, an α-helical linker, an alkyl chain (e.g., an optionally substituted C₁₋₁₂ alkylene or alkynyl chain), or a polyethylene glycol (e.g., (CH₂CH₂O)ₘ, where m is from 1 to 50).

By “micro” is meant having at least one dimension that is less than 1 mm and, optionally, equal to or larger than about 1 μm. For instance, a microstructure (e.g., any structure described herein, such as a microparticle) can have a length, width, height, cross-sectional dimension, circumference, radius (e.g., external or internal radius), or diameter that is less than 1 mm.

By “nano” is meant having at least one dimension that is less than 1 m but equal to or larger than about 1 nm. For instance, a nanostructure (e.g., any structure described herein, such as a nanoparticle) can have a length, width, height, cross-sectional dimension, circumference, radius (e.g., external or internal radius), or diameter that is less than 1 m but equal to or larger than 1 nm. In other instances, the nanostructure has a dimension that is of from about 1 nm to about 1 μm.

As used herein, the terms “administering,” and “introducing” are used interchangeably and refer to the delivery of a compound or a composition to a subject by any useful method or route, such that a desired effect(s) is produced.

As used herein an “effective amount” or “therapeutically effective amount” means an amount necessary to at least partly attain the desired response, or to delay the onset or inhibit progression or halt altogether, the onset or progression of a particular symptom being treated. The amount varies depending upon the health and physical condition of the subject to be treated, the taxonomic group of subject to be treated, the degree of protection desired, the formulation of the composition, the assessment of the medical situation, and other relevant factors. It is expected that the amount will fall in a relatively broad range that can be determined through routine trials.

As used herein, “modulating” or “to modulate” generally means either reducing or inhibiting the activity of, or alternatively increasing the activity of, a target, such as any described herein, as measured using a suitable in vitro, cellular, or in vivo assay. In particular, “modulating” or “to modulate” can mean either reducing or inhibiting the activity of, or alternatively increasing a (relevant or intended) biological activity of, a target, as measured using a suitable in vitro, cellular, or in vivo assay (which will usually depend on the target involved), by at least 5%, at least 10%, at least 25%, at least 50%, at least 60%, at least 70%, at least 80%, at least 90%, or more, inclusive, compared to activity of the target in the same assay under the same conditions but without the presence of an agent. An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100% or more, inclusive, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as any described herein. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

As used herein, the term “subject” can be a human, non-human primate, horse, pig, rabbit, dog, sheep, goat, cow, cat, guinea pig or rodent. A “patient” or “subject in need thereof” refers to a mammal afflicted with a disease or disorder. The term “patient” includes human and veterinary subjects.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The phrase “pharmaceutically acceptable excipient” as used herein means a pharmaceutically acceptable material, composition, carrier, or vehicle, such as a liquid or solid filler, diluent, solvent, media, encapsulating material, manufacturing aid (e.g., lubricant, talc magnesium, calcium or zinc stearate, or steric acid), or solvent encapsulating material, involved in maintaining the stability, solubility, or activity of, an agent for modulating any target described herein or treating any disease described herein. Each excipient must is “acceptable” in the sense of being compatible with the other ingredients of the formulation and not injurious to the patient. Such excipients can refer to an organic or inorganic ingredient, natural or synthetic inactive ingredient in a formulation, with which one or more active ingredients are combined. Excipients can include sterile aqueous or nonaqueous solutions, dispersions, suspensions, or emulsions, as well as sterile powders for reconstitution into sterile injectable solutions or dispersions just prior to use. Non-limiting examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions and by the use of surfactants. These compositions can also contain additives such as preservatives, wetting agents, emulsifying agents, and dispersing agents. Prevention of the action of microorganisms can be ensured by the inclusion of various antibacterial and antifungal agents, such as paraben, chlorobutanol, phenol, sorbic acid and the like. It can also be desirable to include isotonic agents, such as sugars, sodium chloride, and the like. Prolonged absorption of the injectable pharmaceutical form can be brought about by the inclusion of agents, such as aluminum monostearate and gelatin, which delay absorption. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

By “salt” is meant an ionic form of a compound or structure (e.g., any formulas, compounds, or compositions described herein), which includes a cation or anion compound to form an electrically neutral compound or structure. Salts are well known in the art. For example, non-toxic salts, pharmaceutically acceptable salts are described in Berge S M et al., “Pharmaceutical salts,” J. Pharm. Sci. 66(1), 1-19 (1977); and in “Handbook of Pharmaceutical Salts: Properties, Selection, and Use,” Wiley-VCH, April 2011 (2nd rev. ed., eds. PH Stahl and CG Wermuth). The salts can be prepared in situ during the final isolation and purification of the compounds of the invention or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecylsulfate, edetate, ethanesulfonate, fumarate, glucoheptonate, glucomate, glutamate, glycerophosphate, hemisulfate, heptonate, hexanoate, hydrobromide, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2-naphthalenesulfonate, nicotinate, nitrate, oleate, oxalate, palmitate, pamoate, pectinate, persulfate, 3-phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, diethanolamine, ethylenediamine, methylglucamine, and procaine.

The terms “polynucleotide” and “nucleic acid,” used interchangeably herein, refer to a polymeric form of nucleotides of any length, either ribonucleotides or deoxyribonucleotides. Thus, this term includes, but is not limited to, single-stranded (e.g., sense or antisense), double-stranded, or multi-stranded ribonucleic acids (RNAs), deoxyribonucleic acids (DNAs), threose nucleic acids (TNAs), glycol nucleic acids (GNAs), peptide nucleic acids (PNAs), locked nucleic acids (LNAs), or hybrids thereof, genomic DNA, cDNA, DNA-RNA hybrids, or a polymer comprising purine and pyrimidine bases or other natural, chemically or biochemically modified, non-natural, or derivatized nucleotide bases. Polynucleotides can have any useful two-dimensional or three-dimensional structure or motif, such as regions including one or more duplex, triplex, quadruplex, hairpin, and/or pseudoknot structures or motifs.

The term “modified,” as used in reference to nucleic acids, means a nucleic acid sequence including one or more modifications to the nucleobase, nucleoside, nucleotide, phosphate group, sugar group, and/or internucleoside linkage (e.g., phosphodiester backbone, linking phosphate, or a phosphodiester linkage). Yet other non-limiting modifications can include anti-reverse cap analog (ARCA) capping; enzymatic polyadenylation to add a tail of 100-250 adenosine residues; and/or substitution of one or both of cytidine with 5-methylcytidine and/or uridine with pseudouridine.

The nucleoside modification may include, but is not limited to, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

A sugar modification may include, but is not limited to, a locked nucleic acid (LNA, in which the 2′-hydroxyl is connected by a C₁₋₆ alkylene or C₁₋₆ heteroalkylene bridge to the 4′-carbon of the same ribose sugar), replacement of the oxygen in ribose (e.g., with S, Se, or alkylene, such as methylene or ethylene), addition of a double bond (e.g., to replace ribose with cyclopentenyl or cyclohexenyl), ring contraction of ribose (e.g., to form a 4-membered ring of cyclobutane or oxetane), ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone), multicyclic forms (e.g., tricyclic), and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with a-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

A backbone modification may include, but is not limited to, 2′-deoxy- or 2′-O-methyl modifications. A phosphate group modification may include, but is not limited to, phosphorothioate, phosphoroselenates, boranophosphates, boranophosphate esters, hydrogen phosphonates, phosphoramidates, phosphorodiamidates, alkyl or aryl phosphonates, phosphotriesters, phosphorodithioates, bridged phosphoramidates, bridged phosphorothioates, or bridged methylene-phosphonates.

By “protein,” “peptide,” or “polypeptide,” as used interchangeably, is meant any chain of more than two amino acids, regardless of post-translational modification (e.g., glycosylation or phosphorylation), constituting all or part of a naturally occurring polypeptide or peptide, or constituting a non-naturally occurring polypeptide or peptide, which can include coded amino acids, non-coded amino acids, modified amino acids (e.g., chemically and/or biologically modified amino acids), and/or modified backbones.

The term “modified,” as used in reference to amino acids, means an amino acid including one or more modifications, such as a post-translation modification (e.g., acetylation, methylation, phosphorylation, ubiquitination, sumoylation, ribosylation, glycosylation, acylation, or isomerization), or including a non-natural amino acid. Such modifications can also include one or more amino acid substitution, as compared to the reference sequence for the protein.

The term “fragment” is meant a portion of a nucleic acid or a polypeptide that is at least one nucleotide or one amino acid shorter than the reference sequence. This portion contains, preferably, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, 1000, 1250, 1500, 1750, 1800 or more nucleotides; or 10, 20, 30, 40, 50, 60, 70, 80, 90, 100, 150, 200, 250, 300, 350, 400, 450, 500, 550, 600, 640 amino acids or more. In another example, any polypeptide fragment can include a stretch of at least about 5 (e.g., about 10, about 20, about 30, about 40, about 50, or about 100) amino acids that are at least about 40% (e.g., about 50%, about 60%, about 70%, about 80%, about 90%, about 95%, about 87%, about 98%, about 99%, or about 100%) identical to any of the sequences described herein can be utilized in accordance with the invention. In certain embodiments, a polypeptide to be utilized in accordance with the invention includes 2, 3, 4, 5, 6, 7, 8, 9, 10, or more mutations (e.g., one or more conservative amino acid substitutions, as described herein). In yet another example, any nucleic acid fragment can include a stretch of at least about 5 (e.g., about 7, about 8, about 10, about 12, about 14, about 18, about 20, about 24, about 28, about 30, or more) nucleotides that are at least about 40% (about 50%, about 60%, about 70%, about 80%, about 90%, about 95%, about 87%, about 98%, about 99%, or about 100%) identical to any of the sequences described herein can be utilized in accordance with the invention. For any sequence or SEQ ID NO described herein for a nucleic acid or for a polypeptide, fragments of these sequences are encompassed by the present disclosure.

The term “conservative amino acid substitution” refers to the interchangeability in proteins of amino acid residues having similar side chains (e.g., of similar size, charge, and/or polarity). For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamic acid and aspartic acid; and a group of amino acids having sulfur containing side chains consists of cysteine and methionine. Exemplary conservative amino acid substitution groups are valine-leucine-isoleucine, phenylalanine-tyrosine, lysine-arginine, alanine-valine, glycine-serine, glutamate-aspartate, and asparagine-glutamine. For any sequence or SEQ ID NO described herein for a polypeptide, conservative amino acid substitutions of these sequences are encompassed by the present disclosure.

As used herein, when a polypeptide or nucleic acid sequence is referred to as having “at least X % sequence identity” to a reference sequence, it is meant that at least X percent of the amino acids or nucleotides in the polypeptide or nucleic acid are identical to those of the reference sequence when the sequences are optimally aligned. An optimal alignment of sequences can be determined in various ways that are within the skill in the art, for instance, the Smith Waterman alignment algorithm (Smith T F et al., J. Mol. Biol. 147, 195-197 (1981)) and BLAST (Basic Local Alignment Search Tool; Altschul S F et al., J. Mol. Biol. 215, 403-410 (1990)). These and other alignment algorithms are accessible using publicly available computer software such as “Best Fit” (Smith T F et al., Adv. Appl. Math. 2(4), 482-489 (1981)) as incorporated into GeneMatcher Plus™ (Schwartz and Dayhoff, “Atlas of Protein Sequence and Structure,” ed. Dayhoff, M. O., pp. 353-358, 1979), BLAST, BLAST-2, BLAST-P, BLAST-N, BLAST-X, WU-BLAST-2, ALIGN, ALIGN-2, CLUSTAL, T-COFFEE, MUSCLE, MAFFT, or Megalign (DNASTAR). In addition, those skilled in the art can determine appropriate parameters for measuring alignment, including any algorithms needed to achieve optimal alignment over the length of the sequences being compared. In general, for polypeptides, the length of comparison sequences can be at least five amino acids, preferably 10, 20, 30, 40, 50, 60, 70, 80, 90, 100, 125, 150, 175, 200, 250, 300, 400, 500, 600, 700, or more amino acids, up to the entire length of the polypeptide. For nucleic acids, the length of comparison sequences can generally be at least 10, 20, 30, 40, 50, 60, 70, 80, 90, 100, 125, 150, 175, 200, 250, 300, 400, 500, 600, 700, 800, 900, 1000, 1100, 1200, 1300, 1400, 1500, 1600, 1700, 1800, 1900, 2000, 2100, or more nucleotides, up to the entire length of the nucleic acid molecule. It is understood that for the purposes of determining sequence identity when comparing a DNA sequence to an RNA sequence, a thymine nucleotide is equivalent to a uracil nucleotide.

By “substantial identity” or “substantially identical” is meant a polypeptide or nucleic acid sequence that has the same polypeptide or nucleic acid sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least about 50%, 60%, 70%, 75%, 80%, 85%, 90%, 95%, 96%, 97%, 98%, 99%, or 100% sequence identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). For nucleic acids, the length of comparison sequences will generally be at least 5, 10, 11, 12, 13, 14, 15, 16, 17, 18, 19, 20, 21, 22, 23, 24, or 25 contiguous nucleotides (e.g., the full-length nucleotide sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis., 53705). Such software may match similar sequences by assigning degrees of homology to various substitutions, deletions, and other modifications.

As used herein, the term “about” means+/−10% of any recited value. As used herein, this term modifies any recited value, range of values, or endpoints of one or more ranges.

As used herein, the terms “top,” “bottom,” “upper,” “lower,” “above,” and “below” are used to provide a relative relationship between structures. The use of these terms does not indicate or require that a particular structure must be located at a particular location in the apparatus.

Other features and advantages of the present disclosure will be apparent from the following detailed description, the figures, and the claims.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows CXCR4-targeted nanoparticles (NPs) for p53 mRNA delivery to hepatocellular carcinoma (HCC). a: Schematic of CXCR4-targeted p53 mRNA NPs and combinatorial strategy using anti-PD-1 therapy to reprogram the immunosuppressive tumor microenvironment for effective treatment of p53-deficient HCC. The combination of CTCE-p53 NPs and PD-1 blockade effectively and globally reprogrammed the immune TME of HCC, as indicated by activation of CD8⁺ T cells and NK cells, favorable polarization of TAMs towards the anti-tumor phenotype, and increased expression of anti-tumor cytokines. b: Flow cytometric analysis of cellular uptake of CTCE-EGFP mRNA NPs with different CTCE peptide densities versus SCP-EGFP mRNA NPs with 5% SCP density in RIL-175 HCC cells (n=3 cell samples/group). c: Confocal fluorescence imaging of RIL-175 cell uptake of SCP-Cy5-Luciferase (Luc) mRNA NPs versus CTCE-Cy5-Luc mRNA NPs after 4 h treatment. Scale bar: 100 μm. d: Effect of different cationic lipid-like materials G₀-Cₘ on the transfection efficacy of Luc-mRNA NPs (mRNA concentration: 0.25 g/mL, n=3 samples/group). e: TEM image of CTCE-mRNA NPs. Scale bar: 200 nm. f: Average particle size and zeta potential of the p53 NPs, SCP-p53 NPs, and CTCE-p53 NPs (n=3 samples/group). Data in b, d, and f are presented as mean values±SD. For c and e, a representative image from one of five independent fields of view in a single experiment.

FIG. 2 shows the chemical structure and ¹H-NMR spectra of DSPE-PEG-CTCE (top) and DSPE-PEG-SCP (bottom).

FIG. 3 shows cationic lipid-like compounds. a, c: Chemical structure of non-limiting G₀-Cₘ lipid-like compounds. b: Chemical synthetic route of non-limiting cationic lipid-like compounds.

FIG. 4A-4B shows the chemical structure of other lipid-like compounds, including (A) Go to G₄ compounds and (B) G₅ compounds.

FIG. 5 shows ¹H-NMR spectra of different cationic lipids.

FIG. 6 shows the effect of different cationic lipid-like materials G₀-Cₘ on the transfection efficacy of Luciferase-mRNA NPs (mRNA concentration: 0.5 g/mL). All data are presented as mean±S.D. This experiment was repeated thrice independently with similar results.

FIG. 7 shows the effect of different cationic lipid-like materials G₀-Cₘ on the cellular uptake of Cy5-Luc-mRNA NPs (mRNA concentration: 0.25 g/mL). This experiment was repeated thrice independently with similar results.

FIG. 8 shows (A) average particle size (nm) and (B) zeta potential (mV) of the CTCE-Luc NPs, CTCE-GFP NPs, CTCE-p53 NPs, CTCE-Cy5-Luc NPs, and CTCE-Cy5-GFP NPs (n=3 samples/group). All data are presented as mean±S.D.

FIG. 9 shows characterization of p53-mRNA NPs. a: Agarose gel electrophoresis assay of EGFP-mRNA stability at various weight ratios of G₀-C₈/mRNA (Lane 1: 1; Lane 2: 2; Lane 3: 5; Lane 4: 10; Lane 5: 20) and at naked forms (Lane 6: free mRNA in DMF, Lane 7: free mRNA in 1×PBS buffer). This experiment was repeated thrice independently with similar results. b: Stability of p53-mRNA NP in 10% serum at 37° C. was evaluated by measuring particle size changes with dynamic light scattering at various time points up to 96 h. This experiment was repeated thrice independently with similar results.

FIG. 10 shows stability of p53-mRNA NP in 10% serum at 37° C. evaluated by measuring cell viabilities towards RIL-175 cells at various time points up to 96 h (n=3 cell samples/group). All data are presented as mean±S.D.

FIG. 11 shows EGFP-mRNA complexation ability of G₀-C₈ at different pH and weight ratios of G₀-C₈/mRNA by agarose gel electrophoresis assay (Lane 1: pH 7.4, G₀-C₈/mRNA=2; Lane 2: pH 7.4, G₀-C₈/mRNA=5; Lane 3: pH 7.4, G₀-C₈/mRNA=50; Lane 4: pH 7.4, G₀-C₈/mRNA=200; Lane 5: free mRNA in pH 7.4; Lane 6: free mRNA in pH 3.5; Lane 7: pH 3.5, G₀-C₈/mRNA=2; Lane 8: pH 3.5, G₀-C₈/mRNA=5). This experiment was repeated thrice independently with similar results.

FIG. 12 shows in vitro cell viability of G₀-C₈ at different ratios of G₀-C₈/EGFP mRNA. All data are presented as mean±S.D. (n=3 cell samples/group).

FIG. 13 shows an in vitro toxicity study of Luciferase-mRNA NPs. The viability of the (a)p53-null RIL-175 cells and (b) normal liver cells THLE-3 cells after the treatment with PBS, empty NPs, free Luciferase-mRNA (1 μg/ml), CTCE-Luciferase-mRNA NPs (0.125, 0.25, 0.5, 0.75 or 1 g/mL), Lipo2k (Lipo2k only), and Luciferase-mRNA Lipo2k 1 (Luciferase mRNA transfected by Lipo2k at the mRNA concentration of 1 g/mL), as measured by AlamarBlue assay. All data are presented as mean±S.D. (n=3 cell samples/group).

FIG. 14 shows CXCR4-mediated HCC-targeting of CTCE-mRNA NPs in vitro and in vivo. a: Flow cytornetry analysis of in vitro transfection efficiency (% GFP positive cells) of SCP-EGFP NPs vs. CTCE-EGFP NPs in p33-null RIL-175 cells. b: Immunofluorescence of RIL-175 cells transfected with SCP-EGFP NPs vs. CTCE-EGFP NPs (magnification, ×50). Cells were treated with SCP-EGF P NPs or CTCE-EGFP NPs for 12 h and further incubated for 24 h with fresh cell culture medium (mRNA concentration: 0.5 μg/mL). Scale bar: 100 μm. c: Circulation profile of free Cy5-Luc mRNA, SCP-Cy5-Luc NPs, and CTCE-Cy5-Luc NPs (mRNA dose: 350 μg/kg) after i.v. administration. d, e: Quantification of biodistribution of free Cy5-Luciferase mRNA, SCP-Cy5-Luciferase (Luc) NPs, and CTCE-Cy5-Luc NPs in orthotopic (d) and ectopic (e) HCC grafts (n=3 mice/group) at 24 h post-i.v. injection (mRNA dose: 350 μg/kg). f: Western blot analysis of p53 protein expression after treatments (mRNA concentration: 0.5 μg/mL). β-actin was used as the loading control. g: Immunofluorescence for p53 in RIL-175 cells after treatment with saline or CTCE-p53 NPs (p53 mRNA concentration: 0.25 μg/mL). Scale bar: 50 Lm. h: RIL-175 cell growth rate after treatment with control (saline), CTCE-EGFP NPs, empty NPs, SCP-p53 NPs, or CTCE-p53 NPs (mRNA concentration: 0.5 μg/mL) (n 3 cell samples/group). i: RIL-175 cell viability after treatment with control (saline), empty NPs, control NPs (CTCE-EGFP N-Ps), or CTCE-p53 NPs with different mRNA concentrations (0.0625-0.75 μg/mL) (n=3 cell samples/group). Statistical significance was calculated using one-way ANOVA with a Tukey post-hoc test. Data in c, d, e, h, and i are presented as mean values i S.D. **P<0.01; ***P<0.001; ****P<0.0001. For b and g, a representative image from one of five independent fields of view in a single experiment, For f, this experiment was repeated five times independently with similar results.

FIG. 15 shows low cytometry analysis of in vitro transfection efficiency (% GFP positive cells) of free EGFP mRNA, SCP-EGFP NPs, and CTCE-EGFP NPs in p53-null RIL-175 cells. Mean fluorescent intensity (MFJ) in transfected cells was analyzed by Flowjo. All data are presented as mean±S.D. (n=3 cell samples/group).

FIG. 16 shows (A) fluorescent images of RIL-175 cells treated with CTCE-Cy5-Luciferase mRNA NPs and (B) fluorescent images of RIL-175 cells after blocking the CXCR-4 receptor. This experiment was repeated thrice independently with similar results.

FIG. 17 shows western blotting of the CXCR4 expression of the CXCR4-KO RIL-175 cells (sgRNA2) by CRISPR/Cas9 editing. This experiment was repeated thrice independently with similar results.

FIG. 18 shows (A) fluorescent images of sg control RIL-175 cells treated with CTCE-Cy5-Luciferase mRNA NPs and (right) fluorescent images of CXCR4-KO cell line (sgRNA2 RIL-175 cells). This experiment was repeated thrice independently with similar results.

FIG. 19 shows representative immunofluorescence of CTCE-Cy5-Luciferase mRNA NP cellular uptake at different time points. This experiment was repeated thrice independently with similar results.

FIG. 20 shows representative biodistribution of free Cy5-Luciferase mRNA (F), non-targeted Cy5-Luciferase mRNA NP (NT), and CTCE targeted Cy5-Luciferase-mRNA NP (T) in different organs including tumors from C57BL/6 mice bearing RIL-175 tumor 24 h post-injection (mRNA dosage: 350 μg/kg). Results are provided for (a) orthotopic RIL-175 HCC model and (b) s.c. grafted RIL-175 HCC model. Representative image from one of three independent experiments.

FIG. 21 shows HCA-1 cell viability after treatment with control (saline), Empty NPs, Control NPs (CTCE-EGFP NPs), or CTCE-p53 NPs with different mRNA concentrations (0.25 and 0.5 g/mL, respectively). All data are presented as mean±S.D. (n=3 cell samples/group).

FIG. 22 shows PD-1 blockade combined with CXCR4-targeted p53 mRNA NPs reprograms the immune TME and promotes anti-tumor immunity in HCC. a: Timeline of tumor implantation and treatment schedule in the orthotopic HCC model. The mice with orthotopic RIL-175 tumor were treated with CTCE-EGFP mRNA NPs or CTCE-p53 mRNA NPs every 3 days for 4 i.v. injections. Anti-PD-1 (aPD1) was given at 10 mg/kg every 3 days by i.p. injection. b: High-frequency ultrasound images of the RIL-175 orthotopic tumor-bearing C57BL/6 mice at Day 7, 10, 13, 16, and 19 (n=7 mice/group). c, d: Tumor growth profile of each indicated treatment group (n=7 mice/group). e: Immunofluorescence staining of p53 expression in RIL-175 tumors (red signals) in different groups. Scale bar: 200 μm. f-n: Flow cytometry analysis (n=7 samples for CTCE-EGFP-NPs and aPD1 group; n=6 samples for CTCE-p53 NPs and CTCE-p53 NPs+aPD1 group) of tumor CD8⁺ cytotoxic T cells (f), IFN-g⁺TNF-α⁺ cells among CD8⁺ T cells (g), CD4⁺ T cells (h), CD11b⁺ cells when gating on NK cells (i), KLRG1⁺ cells when gating on CD11b⁺ NK cells (j), IFN-g⁺ cells when gating on NK cells (k), IFN-gR⁺ cells when gating on NK cells (1), M1-like tumor-associated macrophages (TAMs) (m), and M2-like TAMs (n). o-q: Increased levels of expression of TNF-α (o), IL-1β (p), and IFN-γ (q) in RIL-175 tumor tissues by protein array measurements after combination treatment (n=4 tumor samples/group). Statistical significance was calculated via one-way ANOVA with a Tukey post-hoc test. All data are presented as mean±S.E.M. For e, this experiment was repeated thrice independently with similar results. *P<0.05; **P<0.01; ***P<0.001; ****P<0.0001.

FIG. 23 shows PD1 blockade combined with CXCR4-targeted p53 mRNA NPs inhibits tumor growth and reprograms the immune TME. a: Tumor weight after 4 injections of CTCE-EGFP mRNA NPs (i.v.) and/or anti-PD1 (aPD1) antibody (10 mg/kg, i.p.) every 3 days (n=7 mice for CTCE-EGFP-NPs and aPDI group; n=6 mice for CTCE-p53 NPs and CTCE-p53 NPs+aPD1 group). b-d: Multiplexed array analysis of tumor tissue for expression levels of IL-10 (b), IL-6 (c), and MCP1 (CCL2) (d) (n=4 tumor samples for each group). *p<0.05 from one-way ANOVA with a Tukey post-hoc test. All data are presented as mean±S.E.M.

FIG. 24 shows the comparison of the therapeutic efficacy between the combination of CTCE-p53-mRNA NPs with anti-PD-1 (aPD1) and the combined treatment of anti-PD-L1 (aPD-L1) and anti-VEGFR2 (DC101) in orthotopic HCC model in C57BL/6 mice. a: Timeline of tumor implantation and treatment schedule for survival studies in RIL-175 orthotopic murine HCC model. To develop the RIL-175 orthotopic model, approximately 6×10⁵ RIL-175 cells 1:1 in Matrigel (Mediatech/Corning, Manassas, VA) were grafted into the left extrahepatic lobe of C57B1/6 mice (6-8 weeks old). b, c: Tumor growth kinetics in each treatment group measured by ultrasound imaging, data are presented as mean±S.E.M. d: Survival distributions in each treatment group. For b-d, day 0: the time for the first treatment. VEGFR2: Vascular Endothelial Growth Factor Receptor 2. Statistical significance was analyzed via one-way ANOVA with a Tukey post-hoc test. The statistical method for survival analysis is Logrank test. *P<0.05; **P<0.01; ****P<0.0001; n=12 mice/group.

FIG. 25 shows the therapeutic efficacy of the combination of CTCE-p53-mRNA NPs with anti-PD-1 (aPD1) in C3H mice via orthotopic HCA-1 HCC model. a: Timeline of tumor implantation and treatment schedule for survival studies in HCC models. b: Survival data from the HCA-1 orthotopic mice model (n=12). c, d: Tumor growth profile of each indicated treatment group (n=12 mice for CTCE EGFP-NPs and aPD1 group; n=11 mice for CTCE-p53 NPs and CTCE-p53 NPs+aPD1 group). Statistical significance was analyzed via one-way ANOVA with a Tukey post-hoc test. Data are presented as mean±S.E.M. *P<0.05; **P<0.01; ****P<0.0001.

FIG. 26 shows anti-tumor efficacy of p53-mRNA NPs with anti-PD-1 in a p53-null ectopically (subcutaneously) grafted RIL-175 HCC model. a: Experimental design and treatment schedule (n=12 mice/group). b: Individual tumor growth curves for mice treated with indicated formulations (n=7 mice/group). c: Representative immunofluorescence for p53 protein expression in tumor tissues after different treatments. Scale bar: 200 m. This experiment was repeated thrice independently with similar results.

FIG. 27 shows combining CXCR4-targeted p53 mRNA NPs with PD-1 blockade reprograms the immune TME and promotes antitumor immunity in ectopic HCC. a: Bioluminescence images of the luciferase-expressing RIL-175 tumors grafted subcutaneously in C57B1/6 mice after 6, 12, and 18 days of treatment (n=3 mice/group). b: Tumor growth rate in each treatment group (n=7 mice/group; ***P<0.001). c: Western blotting analysis on the expression levels of p53 protein in the s.c. RIL-175 tumors after treatment. GAPDH was used as the loading control. d-f: Flow cytometry analysis (n=3 tumor samples from each group) of lymph node CD80⁺CD86⁺ dendritic cells gating on CD11c⁺ cells (d) and tumor-infiltrating CD8⁺CD3⁺ T cells (e) and M2-like CD206⁺F4/80⁺CD11 b⁺ macrophages (f). g: Representative immunofluorescence for CD8 (in red) to confirm intratumoral T cell infiltration after treatment with CTCE-EGFP NPs, anti-PD-1 (aPD1), CTCE-p53 NPs, or the combination. Scale bar: 200 μm. h-k: Protein array analysis of differential expression of cytokines in s.c. HCC tissues after treatment (n=3 samples per group): TNF-α (h), IL-10 (i), IFN-7 (j), and IL-6 (k). Statistical significance was calculated using one-way ANOVA with a Tukey post-hoc test. All data are presented as mean±S.D. For c and g, this experiment was repeated thrice independently with similar results. *P<0.05; **P<0.01; ***P<0.001; ****P<0.0001.

FIG. 28 shows protein array analysis of cytokines tumor tissues after treatment in the s.c. grafted HCC model (n=3 tumor samples for each group): (a) IL-2, (b) IL-10, and (c) MCP-1. Statistical significance was calculated via one-way ANOVA with a Tukey post-hoc test. All data are presented as mean S.E.M. *P<0.05; **P<0.01; ***P<0.001.

FIG. 29 shows western blot analysis of MHC-1 expression in RIL-175 tumor cell line after p53 NPs treatment at p53 mRNA concentration of 0.25 g/mL (p53 NP 0.25) and 0.5 g/mL (p53 NP 0.5), respectively. This experiment was repeated thrice independently with similar results.

FIG. 30 shows immunofluorescence microscopic images of MHC class 1 expression in RIL-175 tumor cell line after p53 mRNA NPs treatment at p53 mRNA concentration of 0.25 g/mL and 0.5 g/mL, respectively. This experiment was repeated thrice independently with similar results.

FIG. 31 shows therapeutic efficacy of the combination of CTCE-p53-mRNA NPs with anti-PD-1 (aPDI) in orthotopic HCC model. a: Timeline of tumor implantation and treatment schedule for survival studies in HCC models. b, c: Tumor growth profile of each indicated treatment group (n=12 mice/group). d: Survival data from the RIL-175 orthotopic mouse model (n=12 mice/group). e, f: The combination of CTCE-p53-mRNA NPs with aPD1 reduces ascites (e) and pleural effusion (f). g: The combination of CTCE-p53-mRNA NPs with aPD1 reduces lung metastasis (n=12 mice for each group). Statistical significance was calculated via one-way ANOVA with a Tukey post-hoc test. All data are presented as mean S.E.M. *P<0.05; **P<0.01.

FIG. 32 shows average body weight of RIL-175 orthotopic tumor-bearing mice over the course of therapy. Error bars represent body weight in grams S.E.M. (n=12 mice/group).

FIG. 33 shows average body weight of RIL-175 s.c. tumor-bearing mice over the course of therapy. Error bars represent the S.E.M. (n=7 mice/group).

FIG. 34 shows in vivo safety of CTCE-p53 NPs and the combination with anti-PD-1 antibody. a-k: Serum biochemistry analysis (n=4 samples for CTCE-EGFP NPs group; n=5 samples for the left four groups). I-r: Whole blood panel tests analysis (n=5 samples for each group). All data are presented as mean S.E.M.

FIG. 35 shows in vivo toxicity studies. a-h: Hematological analysis based on serum biochemistry and whole blood panel tests. The following parameters were examined: alanine aminotransferase (ALT) (a), aspartate aminotransferase (AST) (b), creatinine (c), urea nitrogen (BUN) (d), glucose (e), carbon dioxide (f), albumin (g), and total protein (h) in HCC tissues in the s.c. RIL-175 model (n=3 mice/group). All data are presented as mean S.E.M.

FIG. 36 shows representative H&E staining of heart, liver, spleen, kidney, and lung tissues after treatment with PBS, CTCE-EGFP NPs, aPDI, CTCE-p53 NPs, and CTCE-p53 NPs combined with aPD1 in orthotopic RIL-175 tumor model. Scale bar: 100 m. This experiment was repeated thrice independently with similar results.

FIG. 37 shows representative H&E staining of heart, liver, spleen, kidney, and lung tissues after treatment with PBS, CTCE-EGFP NPs, aPD1, CTCE-p53 NPs, and CTCE-p53 NPs combined with aPD1 in s.c. RIL-175 tumor model. Scale bar: 200 m. This experiment was repeated thrice independently with similar results.

FIG. 38 shows a non-limiting flow cytometry gating strategy.

FIG. 39 shows the effect of different ionizable lipid-like materials Gₘ-Cₙ on the translation efficacy of Luc-mRNA NPs (mRNA concentration: 0.25 g/mL, n=6 samples/group).

FIG. 40 shows RIL-175 cell viability after treatment with empty NPs, control NPs (EGFP-mRNA NPs), p53 mRNA hybrid polymer-lipid nanoparticles (HNPs), or p53 mRNA lipid nanoparticles (LNPs) with different mRNA concentrations (0.0625-0.5 g/mL) (n=3).

FIG. 41A-41B shows the effects on cell viability/clonogenic survival after in vitro treatment with p53-mRNA NPs and/or radiation of p53-KO HCC cells. Provided are data for (A) murine RIL-175 HCC cells and (B) human Hep3B HCC cells. For (A), the concentration of p53 mRNA NPs mRNA was 0.5 μg/mL, and the dose of radiation was 6 Gy. For (B), the concentration of p53 mRNA NPs mRNA was 0.5 g/mL, and the dose of radiation was 8 Gy.

FIG. 42 shows the effects on tumor growth and mouse survival after in vivo treatment with p53 mRNA NPs and/or radiation of established p53-KO HCC cells. The concentration of p53 mRNA NPs mRNA was 6.5 μg per mice every 3 days for 15 days, and the dose of radiation was 8 Gy daily for 3 days.

FIG. 43 shows the effects on tumor morbidity and metastasis after in vivo treatment with p53-mRNA NPs and/or radiation of established p53-KO HCC cells.

### DETAILED DESCRIPTION

The present disclosure relates to compositions including a lipid-like compound of Gₘ-Cₙ. In use, the compositions can serve as delivery vehicles for a cargo (e.g., any described herein). In non-limiting embodiments, the composition can include a core and an outer layer surrounding the core, in which the core can include a cargo that is complexed with a Gₘ-Cₙ compound. Optionally, the core can further include a polymer (e.g., a water-insoluble polymer, such as any described herein). In some instances, the polymer may be absent from the core. The outer layer can have any useful lipid or a combination of lipids (e.g., any lipid described herein).

In some embodiments, the composition forms a lipid particle (e.g., a lipid nanoparticle or a lipid microparticle), a hybrid polymer-lipid particle (e.g., a hybrid polymer-lipid nanoparticle or a hybrid polymer-lipid microparticle), a solid lipid particle (e.g., a solid lipid nanoparticle or a solid lipid microparticle), a liposome having a bilayer or a multilayer, a micelle, and the like. In one embodiment, the lipid particle includes a cargo (e.g., mRNA), a lipid-like compound, a lipid, a pegylated lipid, and cholesterol. In another embodiment, the hybrid polymer-lipid particle includes a cargo (e.g., mRNA), a lipid-like compound, a water-insoluble polymer, and a lipid (e.g., pegylated lipid or a non-pegylated lipid). The lipid particle and the hybrid polymer-lipid particle can include, e.g., a target ligand.

In some aspects, the present disclosure describes compositions having a particular combinations of cargos and targeting ligands to target certain cells. For example and without limitation, the cargo can encode a protein, and the targeting ligand can be configured to bind to target cells lacking that protein. In one non-limiting embodiment, the composition includes:
a core having a tumor suppressor-encoding mRNA; a lipid-like compound of Gₘ-Cₙ, wherein m≥0 and n<20; and an optional water-insoluble polymer (e.g., PLGA); and
an outer layer surrounding the core, in which the outer layer has: a targeting ligand (e.g., any described herein); and an optional lipid (e.g., a pegylated lipid and/or a non-pegylated lipid).

The mRNA and targeting ligand can be chosen to provide selective delivery of the mRNA cargo to a target cell. For instance and without limitation, the mRNA can be a p53-encoding mRNA to promote translation of p53 in a p53-deficient cell, and the targeting ligand can be selected to provide targeting of that p53-deficient cell. In non-limiting embodiments, the targeting ligand can bind to chemokine receptors present on p53-deficient cells (e.g., targeting ligand for a CXCR4 receptor).

The polymer may be present or absent within the core. For instance, when present, the polymer can be used in combination with a lipid-like compound of Gₘ-Cₙ(e.g., any described herein). With such a core, any useful component can be used within the outer layer. In some embodiments, the outer layer can include one or more lipids to form an outer layer disposed around the polymeric core, thereby forming a hybrid polymer-lipid particle. Optionally, the outer layer can include a targeting ligand.

In other aspects, the present disclosure describes lipid-like compounds of Gₘ-Cₙ to complex a cargo within a core of a delivery vehicle. For example and without limitation, Gₘ-Cₙ compounds having any useful m and n values can be employed with a cargo. A Gₘ-Cₙ compound includes a dendrimer having an m^{th} generation (Gₘ) central structure with terminal capping to provide aliphatic groups having an n number of carbon atoms (Cₙ).

The central structure can include any useful repeating, branched structure. In one non-limiting embodiment, the central structure includes a poly(amidoamine) (PAMAM) formed from a diamine (e.g., alkyleneamine, such as ethylenediamine) that is reacted with a polymerizable monomer (e.g., a vinyl, a vinyl ester, an acetate ester, an acrylate, an acrylate ester, an alkyl acrylate, such as methyl acrylate, ethyl acrylate, butyl acrylate, and the like). By using diamines and monomers in successive cycles, further repeating, branched structures of further generations can be formed. In some embodiments, m in Gₘ is 0, 1, 2, 3, 4, 5, or more.

In turn, aliphatic groups having a reactive moiety can be used to cap or further functionalize the dendrimeric structure. For example and without limitation, a PAMAM structure can include a reactive amino group, which can be further reacted with an aliphatic-containing compound (e.g., R^{A}—X, in which R^{A} is an optionally substituted aliphatic group and X is a reactive group or a leaving group, such as an ester group, an epoxide or oxiranyl group, an acyl group, an acyl halide group, a halogen, a carboxyl group, a carbonyl-containing group, an alkoxy group, and the like). The aliphatic group can have any useful number of carbons (e.g., any described herein, such as from C₁₋₂₀ or ranges therebetween). In some embodiments, n in Cₙ is at least 5, 6, 7, 8, 9, 10, 11, 12, 13, 14, 15, 16, 17, 18, 19, 20, or more.

Any useful combination of m and n can be employed in Gₘ-Cₙ. Non-limiting m and n values can include, e.g., any integer in which m≥0 and n<20; m=0 and n<14; or m>0 and n<20. Other Gₘ-Cₙ compounds, m values, and n values are described herein.

Certain combinations of m and n can provide useful compositions. In some embodiments, the composition includes:
a core, wherein the core includes: a nucleic acid (e.g., an mRNA); a lipid-like compound of Gₘ-Cₙ, wherein (i) m=0 and n<14, (ii) m>0 and n<20, or (iii) m>0 and n<20; and an optional water-insoluble polymer (e.g., PLGA or any described herein); and
an outer layer surrounding the core, wherein the outer layer includes a lipid, a pegylated lipid (e.g., DSPE-PEG), or any lipid described herein.

In certain embodiments, the core is formed by complexing the cargo with a complexing agent (e.g., any lipid-like compound or amphiphile described herein). The core can include one or more cargos (e.g., one or more mRNAs) and one or more lipid-like compounds (e.g., any described herein). A polymer may or may not be included within the core. For instance and without limitation, the core may include one or more cargos (e.g., mRNAs), lipid-like compounds (e.g., Gₘ-Cₙ compounds), and polymers (e.g., water-insoluble polymers). Such a polymeric core may be used with an outer lipid layer to form a hybrid polymer-lipid particle. When the polymer is absent, then the core can be used with an outer lipid layer to from a lipid particle.

For any composition herein, the outer layer can be disposed around the core and include any useful lipid or combination of lipids. Non-limiting lipids can include fatty acids, phospholipids, glycerides, eicosanoids, sphingolipids, steroids, pegylated forms of any of these, as well as combinations thereof. Other non-limiting examples of lipids are described herein.

Optionally, the outer layer can include one or more targeting ligands (e.g., any described herein). In particular embodiments, the targeting ligand includes a targeting moiety (e.g., configured to bind to a target, a target receptor, a target cell, and the like) that is bound to a lipid (e.g., any herein) by way of a linker (e.g., any herein). Non-limiting examples of targeting ligands are described herein.

In particular embodiments, the outer layer includes at least two types of lipids or amphiphiles. In one instance, the outer layer can include a pegylated lipid and a non-pegylated lipid. In another instance, the outer layer can include a non-pegylated lipid and a target ligand, which in turn can be attached to a lipid having a PEG-based linker. In yet another instance, the outer layer can include a pegylated lipid and a target ligand, which in turn can be attached to a lipid having a PEG-based linker. In another instance, the outer layer can include a non-pegylated lipid, a pegylated lipid, and a target ligand, which in turn can be attached to a lipid having a PEG-based linker.

The outer layer can include one or more components to stabilize the lipid layer (e.g., lipid monolayer, bilayer, or multilayer). Such components can include cholesterol, sitosterol, hydroxycholesterol (e.g., 20α-hydroxycholesterol), a pegylated component (e.g., a pegylated phospholipid, a pegylated cholesterol), and the like.

In some embodiments, the composition forms a particle. As used herein, a particle can refer to any entity having a diameter of less than 1,000 microns (μm). In some embodiments, particles have a diameter of 1,000 nm or less, or 300 nm or less. In some embodiments, particles can be a polymeric particle, non-polymeric particle (e.g., a metal particle, quantum dot, ceramic, inorganic material, bone, etc.), liposomes, micelles, hybrids thereof, and/or combinations thereof.

Particles may be microparticles or nanoparticles. Nanoparticles can be employed depending on the use, such as, e.g., for intertissue application, penetration of cells, and certain routes of administration. The particles may have any desired size for the intended use. The particles may have any diameter from about 10 to 1,000 nm. The particle can have a dimension (e.g., a diameter, radius, length, width, height, or an average of any of these) from about 10 to 900 nm, 10 to 800 nm, 10 to 700 nm, 10 to 600 nm, 10 to 500 nm, 20 to 500 nm, 30 to 500 nm, 40 to 500 nm, 50 to 500 nm, 50 to 400 nm, 50 to 350 nm, 50 to 300 nm, or 50 to 200 nm. In some embodiments the nanoparticles can have a diameter less than about 400 nm, 300 nm, or 200 nm.

In some embodiments, the core and the outer layer forms a nanoparticle. The nanoparticle can have any useful size, such as an average size from about 10 to 500 nm (e.g., from about 10 to 50 nm, 10 to 80 nm, 10 to 100 nm, 10 to 150 nm, 10 to 200 nm, 10 to 300 nm, 10 to 400 nm, 20 to 50 nm, 20 to 80 nm, 20 to 100 nm, 20 to 150 nm, 20 to 200 nm, 20 to 300 nm, 20 to 400 nm, 20 to 500 nm, 50 to 80 nm, 50 to 100 nm, 50 to 150 nm, 50 to 200 nm, 50 to 300 nm, 50 to 400 nm, 50 to 500 nm, 80 to 80 nm, 80 to 100 nm, 80 to 150 nm, 80 to 200 nm, 80 to 300 nm, 80 to 400 nm, 80 to 500 nm, 100 to 150 nm, 100 to 200 nm, 100 to 300 nm, 100 to 400 nm, or 100 to 500 nm).

Nanoparticles may be a variety of different shapes, including but not limited to spheroidal, cubic, pyramidal, oblong, cylindrical, toroidal, and the like. Nanoparticles can comprise one or more surfaces. Exemplary nanoparticles that can be adapted for use include (1) the biodegradable nanoparticles disclosed in U.S. Pat. No. 5,543,158 to Gref et al., (2) the polymeric nanoparticles of U.S. Pat. Pub. No. 2006/0002852 to Saltzman et al., (3) the lithographically constructed nanoparticles of U.S. Pat. Pub. No. 2009/0028910 to DeSimone et al., or (4) the particles disclosed in Int. Pub. No. WO 2018/089688 to Shi et al. Further components within compositions, as well as methods of making and using such compositions, are further described herein.

### Cargo

The compositions herein can be configured to provide any useful cargo. Non-limiting cargo can include a nucleic acid, including RNA, mRNA, siRNA, DNA, guide RNA (gRNA), guide DNA (gDNA), DNA/RNA hybrids, and the like. In non-limiting embodiments, the composition includes one or more cargos (e.g., different types of cargo), and the cargo can be present within the core.

In particular embodiments, the cargo can be a nucleic acid (e.g., mRNA) encoding any useful protein. In some embodiments, the encoded protein is a tumor suppressor. Non-limiting examples of tumor suppressors include p53, phosphatase and tensin homolog on chromosome ten (PTEN), or any known in the art (e.g., see Table 1).

As used herein, a tumor suppressor is a protein that acts to reduce the potential for cancer and tumor formation by modulating cell growth, by negative regulation of the cell cycle, and/or by promoting apoptosis. Thus, loss of a tumor suppressor (e.g., through mutation or dysregulation) can lead to unregulated cell growth and tumor development. Mutations and other alterations that are associated with cancer are known in the art. A number of tumor suppressors are known in the art. See, e.g., Table 1.

TABLE 1

| | Genetic | Associated | GenBank Acc No. | |
|---|---|---|---|---|
| GENE | Alteration(s) | Cancer(s) | mRNA | Protein |
| p53 | Point | Liver | AF307851.1 | AAG28785.1 |
| | mutation, | Lung | NM_000546.5 | NP_000537.3 |
| | deletion | Prostate | | |
| PTEN | Point | Prostate, breast, | AF067844.1 | AAD13528.1 |
| | mutation, | glioblastoma, | | |
| | deletion | melanoma, | | |
| | | pancreatic cancer, | | |
| | | colorectal cancer, | | |
| | | leukemia | | |
| APC | Point | Adenomatous | M74088.1 | AAA03586.1 |
| | mutation, | polyposis and | | |
| | deletion | sporadic colorectal | | |
| | | tumors, gastric | | |
| | | cancer | | |
| ARF | Deletion | Breast carcinomas, | AF208864.1 | AAF64278.1 |
| | | colorectal adenoma, | | |
| | | glioblastoma | | |
| BMPR | Point mutation | Gastrointestinal | NM_009758.4 | NP_033888.2 |
| | | cancer | | |
| BRCA1 | Point mutation | Ductal breast | U14680.1 | AAA73985.1 |
| | | cancers, | | |
| | | Epithelial ovarian | | |
| | | cancers | | |
| E-cadherin | Point mutation | Loss of function | Z13009.1 | CAA78353.1 |
| | | leads to metastasis | | |
| EXT1, 2 | Point | Hereditary multiple | S79639.1, | AAB62283.1 |
| | mutation, | exostoses, also | U62740.1 | AAB07008.1 |
| | deletion, | known as diaphyseal | | |
| | insertion | aclasis | | |
| FBXW7 | Point | Breast cancer | AF411971.1 | AAL06290.1 |
| | mutation, | | | |
| | deletion | | | |
| FH | Point mutation | Hereditary | BC003108.1 | AAH03108.1 |
| | | leiomyomatosis and | | |
| | | renal-cell cancer | | |
| GPC3 | Deletions, | Lung carcinoma | L47125.1 | AAA98132.1 |
| | point mutation | | | |
| HIPK2 | Point mutation | Metastatic bladder | AF208291 | AAG41236.1 |
| | | cancer | | |
| HRPT2 | Point mutation | Hereditary | NM_024529.4 | NP_078805.3 |
| (CDC73) | | hyperparathyroidism- | | |
| | | jaw tumor syndrome, | | |
| | | Malignancy in | | |
| | | sporadic parathyroid | | |
| | | tumors | | |
| INPP4B | Deletion, loss | Epithelial | U96922.1 | AAB72153.1 |
| | of | carcinomas and some | | |
| | heterozygosity, | human basal-like | | |
| | reduced | breast carcinomas | | |
| | expression | | | |
| LKB1 | Point | Human Lung Cancer | U63333.1 | AAB05809.1 |
| | mutation, | (especially NSCLC), | | |
| | deletion | cervical carcinomas | | |
| | | Inherited cancer | | |
| | | disorder Peutz- | | |
| | | Jeghers Syndrome | | |
| MEN1 | Point mutation | Pituitary tumors | U93236.1 | AAC51228.1 |
| MMR | Point | Hereditary non- | NM_002438.3 | NP_002429.1 |
| (MRC1) | mutation, | polyposis colon | | |
| | reduced | cancer | | |
| | expression | | | |
| MUTYH | Point | Lung and ovarian | U63329.1 | AAC50618.1 |
| | mutation, | tumors, and | | |
| | deletion | lymphomas | | |
| NF1 | Point | Juvenile | NM_000267.3 | NP_000258.1 |
| | mutation, | myelomonocytic | | |
| | deletion | leukemia, Watson | | |
| | | syndrome and breast | | |
| | | cancer | | |
| NF2 | Point | Meningioma | L11353.1 | AAA36212.1 |
| | mutation, | Thyroid cancer, | | |
| | deletion | mesothelioma, and | | |
| | | melanoma | | |
| p15, | Point mutation | Colorectal cancer, | AB060808.1 | BAB91133.1 |
| p16 | | leukemia | L27211.1 | AAA92554.1 |
| p57 | Point mutation | Beckwith- | NM_000076.2 | NP_000067.1 |
| (CDKN1C) | | Wiedemann | | |
| | | syndrome | | |
| Ptch | Point mutation | Cell carcinomas of | NM_000264.4 | NP_000255.2 |
| | | the skin, ovarian | | |
| | | fibromas, and | | |
| | | medulloblastomas | | |
| RB1 | Point | Prostate cancer | NM_000321.2 | NP_000312.2 |
| | mutation, | Pituitary | | |
| | deletion | melanotroph tumors | | |
| RECQL4 | Point mutation | Osteosarcoma | AB006532.1 | BAA74453.1 |
| SDH | Point | Paraganglioma, renal | U17248.1 | AAA81167.1 |
| | mutation, | cell carcinoma | | |
| | deletion | | | |
| Smad2/3 | Point | Breast cancer | U65019.1 | AAB17054.1 |
| | mutation, | | BC050743.1 | AAH50743.1 |
| | deletions | | | |
| Smad4 | Point mutation | Pancreatic | U44378.1 | AAA91041.1 |
| | | Gastric Carcinoma | | |
| Su(Fu) | Point | Brain tumor | NM_016169.3 | NP_057253.2 |
| | mutation, | | | |
| | deletion | | | |
| TGFβR | Point mutation | Head and neck | NM_001306210.1 | NP_001293139.1 |
| | | cancers, cervical and | | |
| | | ovarian carcinomas | | |
| TSC1/TSC2 | Point mutation | Tuberous sclerosis | AF013168.1 | AAC51674.1 |
| | | complex | AB014460.1 | BAA32694.1 |
| VHL | Point | Renal carcinomas | NM_000551.3 | NP_000542.1 |
| | mutation, | | | |
| | deletion, | | | |
| | hyper- | | | |
| | methylation | | | |
| WT1 | Point | Haematological | NM_000378.4 | NP_000369.3 |
| | mutation, | malignancies | | |
| | deletion | Pediatric | | |
| | | nephroblastoma | | |
| | | Wilms tumor | | |
| XPA | Point mutation | Bladder cancer | NM_000380.3 | NP_000371.1 |
| XPC | Point | ESCC, gastric cancer | NM_004628.4 | NP_004619.3 |
| | mutations, | | | |
| | splice variants | | | |
| XPD | Point mutation | Glioma, NSCLC, | NM_000400.3 | NP_000391.1 |
| (ERCC2) | | Sarcoma | | |
| α-catenin | Point mutation | Basal-like breast | NM_001323983.1 | NP_001310912.1 |
| (CTNNA1) | | cancer | | |
| RASSF1A | Hyper- | Lung, Cervical | NM_007182.4 | NP_009113.3 |
| | methylation, | Cancer | | |
| | point mutation | | | |
| SDHB | Point mutation | Kidney | NM_003000.2 | NP_002991.2 |
| | | Paragangliomas | | |
| SIN3B | Point mutation | Prostate cancer | NM_015260.3 | NP_056075.1 |
| RGS12 | Point mutation | Prostate cancer | NM_198227.1 | NP_937870.1 |
| Kai 1 | Deletion, | Prostate cancer | NM_002231.3 | NP_002222.1 |
| metastasis | mutation and | | | |
| suppressor | loss of | | | |
| (CD82) | expression | | | |
| ING1B | Point mutation | Prostate cancer, | NM_198218.2 | NP_937861.1 |
| | | Brain tumors | | |
| Atg7 | Deletion | Prostate cancer | NM_001349232.1 | NP_001336161.1 |
| JARID1D | Point mutation | Prostate cancer | NM_001146705.1 | NP_001140177.1 |
| (KDM5D) | | | | |
| PALB2 | Point mutation | Breast cancer | NM_024675.3 | NP_078951.2 |
| TP53BP1 | Point mutation | Breast cancer | NM_001141980.2 | NP_001135452.1 |
| RAD51 | Point mutation | Breast cancer | NM_133487.3 | NP_597994.3 |
| XRCC4 | Point mutation | Breast cancer | NM_003401.4 | NP_003392.1 |
| KEAP1 | Point mutation | Liver cancer | NM_203500.1 | NP_987096.1 |
| KEAP1 | Point mutation | Liver cancer | NM_012289.3 | NP_036421.2 |
| RPS6KA3 | Point mutation | Liver cancer | NM_004586.2 | NP_004577.1 |
| RARβ | Point mutation | Lung cancer | NM_000965.4 | NP_000956.2 |
| FHIT | Point mutation | Lung cancer | NM_002012.3 | NP_002003.1 |
| PTCH1 | Point mutation | Lung cancer | NM_001083602.2 | NP_001077071.1 |
| DCC | Point mutation | Colorectal cancer | NM_005215.3 | NP_005206.2 |
| BAX | Point mutation | Colorectal cancer | NM_001291428.1 | NP_001278357.1 |
| AML1 | Point mutation | Acute myeloid | NM_001754.4 | NP_001745.2 |
| (RUNX1) | | leukemia | | |
| CDKN2A | Point mutation | Bladder | NM_000077.4 | NP_000068.1 |
| CDKN1B | Point mutation | Prostate cancer | NM_004064.4 | NP_004055.1 |
| NKX3-1 | Point mutation | Prostate cancer | NM_006167.3 | NP_006158.2 |
| RPP14 | Point mutation | Melanoma | NM_001098783.2 | NP_001092253.1 |
| CDK4 | Point mutation | Melanoma | NM_000075.3 | NP_000066.1 |
| CDK6 | Point mutation | Melanoma | NM_001259.7 | NP_001250.1 |

The above sequences are exemplary, as some of the above genes may have multiple transcript variants; generally speaking, the methods can include using an mRNA sequence for the variant that is predominantly expressed in a normal, non-cancerous cell of the same type as the tumor. The methods can include using a nucleotide sequence coding for an mRNA that is at least 80% identical to a reference sequence in Table 1. In some embodiments, the nucleotide sequences are at least 85%, 90%, 95%, 99% or 100% identical.

To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 80% (in some embodiments, about 85%, 90%, 95%, or 100%) of the length of the reference sequence. The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch (J. Mol. Biol. 48, 444-453 (1970)) algorithm, which has been incorporated into the GAP program in the GCG software package, using a Blosum 62 scoring matrix with a gap penalty of 12, a gap extend penalty of 4, and a frameshift gap penalty of 5. Other algorithms are disclosed herein.

As disclosed herein, the delivery vehicle (e.g., nanoparticle) can be complexed with one, two, or more mRNAs (e.g., a plurality of mRNAs) that encode a single tumor suppressor or encode multiple tumor suppressors. The selection of the number and type of tumor suppressor can depend on the type of tumor cell being targeted or the type of cancer being treated. In some embodiments, the cancer is liver cancer, and the mRNA is p53. In other embodiments, the cancer is lung cancer, and the mRNA is p53. In yet other embodiments, the cancer is prostate cancer, and the mRNA is p53, PTEN, and/or p53. Other cancers can include any disclosed herein (e.g., a p53-deficient cancer), as well as those provided in Table 1.

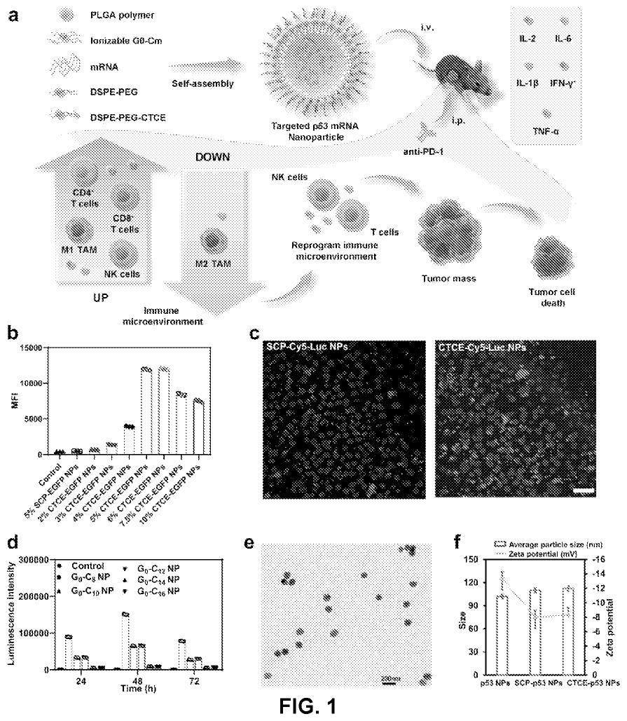

A mature mRNA is generally comprised of five distinct portions (see, e.g., FIG. 1a of Islam et al., Biomater. Sci. 3(12), 1519-1533 (2015)): (i) a cap structure, (ii) a 5′ untranslated region (5′ UTR), (iii) an open reading frame (ORF), (iv) a 3′ untranslated region (3′ UTR), and (v) a poly(A) tail (a tail of 100-250 adenosine residues). Typically, the mRNA will be in vitro transcribed using methods known in the art. The mRNA will typically be modified, e.g., to extend half-life or to reduce immunogenicity. For example, the mRNA can be capped with an anti-reverse cap analog (ARCA), in which OCH₃ is used to replace or remove natural 3′ OH cap groups to avoid inappropriate cap orientation. Tetraphosphate ARCAs or phosphorothioate ARCAs can also be used (see, e.g., Islam et al., (2015)). The mRNA can be enzymatically polyadenylated (addition of a poly adenine (A) tail to the 3′ end of mRNA), e.g., to comprise a poly-A tail of at least 100 or 150 As. Typically, poly(A) polymerase is used. For example, E. coli poly(A) polymerase (E-PAP) I has been optimized to add a poly(A) tail of at least 150 adenines to the 3′ terminal of in vitro transcribed mRNA. In some embodiments, any adenylate-uridylate rice elements (AREs) are removed or replaced with 3′ UTR of a stable mRNA species such as β-globin mRNA. Iron responsive elements (IREs) can be added in the 5′ or 3′ UTR. In some embodiments, the mRNAs include full or partial (e.g., at least 50%, 60%, 70%, 80%, or 90%) substitution of cytidine triphosphate and uridine triphosphate with naturally occurring 5-methylcytidine and pseudouridine (W) triphosphate. See Islam et al., (2015) and references cited therein.

### Amphiphiles

The compositions herein can include one or more amphiphiles, and such amphiphiles can be present in any useful location (e.g., within the core, within the outer layer, between the core and the outer layer, and/or on the outer layer). As used herein, the terms “amphiphile” and “amphiphilic material” are used interchangeably. In certain embodiments, an amphiphile refers to a molecule having both lipophilic and hydrophilic properties. An amphiphile can therefore comprise a segment that is hydrophobic and a segment that is hydrophilic.

In other embodiments, an amphiphile refers to a molecule having both a non-polar portion and a polar portion. Often, an amphiphilic compound has a polar head attached to a long hydrophobic tail. In some embodiments, the polar portion is soluble in water, while the non-polar portion is less soluble or insoluble in water (e.g., as compared to the polar portion). In addition, the polar portion may have either a formal positive charge, or a formal negative charge. Alternatively, the polar portion may have both a formal positive and a negative charge, and be a zwitterion or inner salt. The amphiphilic compound can be, but is not limited to, one or a plurality of the following: naturally derived lipids, surfactants, or synthesized compounds with both hydrophilic and hydrophobic moieties.

A hydrophobic segment of an amphiphile can include, e.g., a hydrocarbon or a hydrocarbon that is substituted exclusively or predominantly with hydrophobic substituents such as halogen atoms. Typically, the hydrophobic segment can comprise a chain of 10, or more (e.g., 10, 11, 12, 13, 14, 15, 16, 17, 18, 19, 20 or more) carbon atoms. In some embodiments, the hydrophobic segment comprises an aliphatic chain (e.g., as defined herein), which in some embodiments can be branched and in some embodiments can be unbranched. In some embodiments, the hydrophobic segment comprises an aliphatic chain that is saturated. In some embodiments, the hydrophobic segment comprises an aliphatic chain that is unsaturated.

A hydrophilic segment of an amphiphile can comprise, e.g., one or more polar groups such as hydroxyl or ether groups. A hydrophilic segment of an amphiphile can comprise, e.g., one or more charged groups. A charged group can include a cation, e.g., ammonium or phosphonium groups. A charged group can include an anion, e.g., phosphate or sulfate or carboxylate groups, as well as deprotonated groups. The hydrophilic segment can be cationic, anionic, or zwitterionic.

Non-limiting amphiphiles can include polymers, dendrimers, lipids (e.g., pegylated lipids, cationic lipids, anionic lipids, neutral lipids, phospholipids, and the like), lipid-like compounds (e.g., Gₙ-Cₘ compounds herein), as well as modified forms thereof.

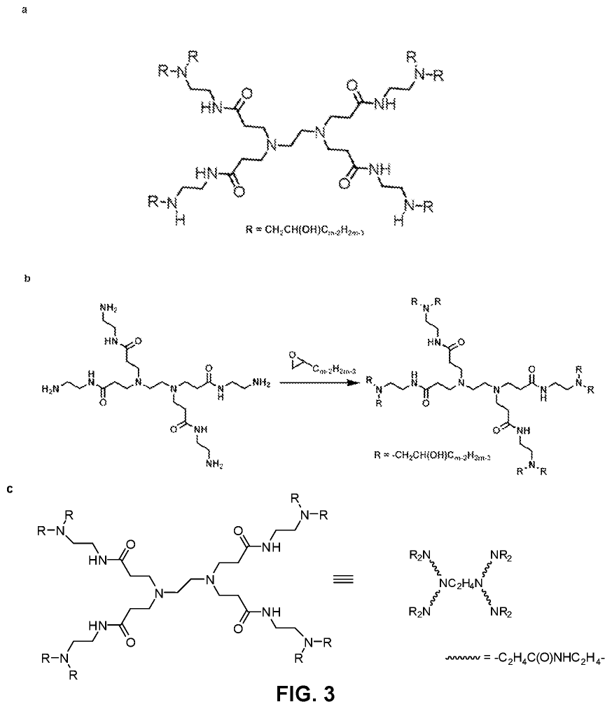

Dendrimers (also known as dendrons, arborols, or cascade molecules) are repetitively branched molecules that can be classified by generation, which refers to the number of repeated branching cycles performed during synthesis. For example, poly(amidoamine) (PAMAM) is ethylenediamine reacted with methyl acrylate, and then another ethylenediamine to make a generation 0 (Go) PAMAM (see, e.g., first reactant in FIG. 3b). Optionally, such dendrimers can be functionalized with one or more aliphatic groups, thereby providing a hydrophilic segment (including amino groups) and a hydrophobic segment (including aliphatic groups). Non-limiting examples of dendrimers can include an amino dendrimer (e.g., ethylenediamine core-poly (amidoamine) (PAMAM) generation 0 dendrimer (G₀), ethylenediamine branched polyethylenimine (Mw ˜800) (PEI), polypropylenimine tetramine dendrimer, generation 1 (DAB), and derivatives thereof, e.g., amino derivatives formed by reacting an amine group with an alkyl epoxide, e.g., G₀-C₁₄ dendrimer described in Xu et al., Proc. Natl Acad. Sci. U.S.A. 110, 18638-18643 (2013), which is hereby incorporated by reference in its entirety).

Lipids can also be considered amphiphiles. In one instance, the amphiphile can be a pegylated lipid, such as a PEG-phospholipid (e.g., 14:0 PEG350 PE (1,2-dimyristoyl-sn-glycero-3-phosphoethanolamine-N-[methoxy(polyethylene glycol)-350]), 14:0 PEG350 PE (1,2-dipalmitoyl-sn-glycero-3-phosphoethanolamine-N-[methoxy(polyethylene glycol)-350]), 18:0 PEG350 PE (1,2-distearoyl-sn-glycero-3-phosphoethanolamine-N-[methoxy (polyethylene glycol)-350]), 18:1 PEG350 PE (1,2-dioleoyl-sn-glycero-3-phosphoethanolamine-N-[methoxy(polyethylene glycol)-350]), 14:0 PEG550 PE (1,2-dimyristoyl-sn-glycero-3-phosphoethanolamine-N-[methoxy(polyethylene glycol)-550]), 14:0 PEG550 PE (1,2-dipalmitoyl-sn-glycero-3-phosphoethanolamine-N-[methoxy (polyethylene glycol)-550]), 18:0 PEG550 PE (1,2-distearoyl-sn-glycero-3-phosphoethanolamine-N-[methoxy(polyethylene glycol)-550]), 18:1 PEG550 PE (1,2-dioleoyl-sn-glycero-3-phosphoethanolamine-N-[methoxy(polyethylene glycol)-550]), 14:0 PEG750 PE (1,2-dimyristoyl-sn-glycero-3-phosphoethanolamine-N-[methoxy(polyethylene glycol)-750]), 14:0 PEG750 PE (1,2-dipalmitoyl-sn-glycero-3-phosphoethanolamine-N-[methoxy(polyethylene glycol)-750]), 18:0 PEG750 PE (1,2-distearoyl-sn-glycero-3-phosphoethanolamine-N-[methoxy(polyethylene glycol)-750]), 18:1 PEG750 PE (1,2-dioleoyl-sn-glycero-3-phosphoethanolamine-N-[methoxy(polyethylene glycol)-750]), 14:0 PEG1000 PE (1,2-dimyristoyl-sn-glycero-3-phosphoethanolamine-N-[methoxy(polyethylene glycol)-1000]), 14:0 PEG1000 PE (1,2-dipalmitoyl-sn-glycero-3-phosphoethanolamine-N-[methoxy(polyethylene glycol)-1000]), 18:0 PEG1000 PE (1,2-distearoyl-sn-glycero-3-phosphoethanolamine-N-[methoxy(polyethylene glycol)-1000]) (DSPE-PEG1K), 18:1 PEG1000 PE (1,2-dioleoyl-sn-glycero-3-phosphoethanolamine-N-[methoxy(polyethylene glycol)-1000]), 14:0 PEG2000 PE (1,2-dimyristoyl-sn-glycero-3-phosphoethanolamine-N-[methoxy(polyethylene glycol)-2000]), 14:0 PEG2000 PE (1,2-dipalmitoyl-sn-glycero-3-phosphoethanolamine-N-[methoxy(polyethylene glycol)-2000]), 18:0 PEG2000 PE (1,2-distearoyl-sn-glycero-3-phosphoethanolamine-N-[methoxy(polyethylene glycol)-2000]) (DSPE-PEG2K), 18:1 PEG2000 PE (1,2-dioleoyl-sn-glycero-3-phosphoethanolamine-N-[methoxy(polyethylene glycol)-2000]), 14:0 PEG3000 PE (1,2-dimyristoyl-sn-glycero-3-phosphoethanolamine-N-[methoxy(polyethylene glycol)-3000]), 14:0 PEG3000 PE (1,2-dipalmitoyl-sn-glycero-3-phosphoethanolamine-N-[methoxy(polyethylene glycol)-3000]), 18:0 PEG3000 PE (1,2-distearoyl-sn-glycero-3-phosphoethanolamine-N-[methoxy (polyethylene glycol)-3000]) (DSPE-PEG3K), 18:1 PEG3000 PE (1,2-dioleoyl-sn-glycero-3-phosphoethanolamine-N-[methoxy(polyethylene glycol)-3000]), 14:0 PEG5000 PE (1,2-dimyristoyl-sn-glycero-3-phosphoethanolamine-N-[methoxy(polyethylene glycol)-5000]), 14:0 PEG5000 PE (1,2-dipalmitoyl-sn-glycero-3-phosphoethanolamine-N-[methoxy (polyethylene glycol)-5000]), 18:0 PEG5000 PE (1,2-distearoyl-sn-glycero-3-phosphoethanolamine-N-[methoxy(polyethylene glycol)-5000]) (DSPE-PEG5K), 18:1 PEG5000 PE (1,2-dioleoyl-sn-glycero-3-phosphoethanolamine-N-[methoxy(polyethylene glycol)-5000]), 1,2-dimyristoyl-rac-glycero-3-methoxypolyethylene glycol (DMG-PEG), 1,2-dimyristoyl-rac-glycero-3-methoxypolyethylene glycol-2000 (DMG-PEG2K), distearoyl-rac-glycerol-PEG2K (DSG-PEG2K), methoxypolyethyleneglycoloxy (2000)-N,N-ditetradecylacetamide (ALV-0159), and the like). Alternatives of pegylated lipids may also be used, such as, e.g., N,N-ditetradecyl-polysarcosine-25 (N-Tetamine-pSar25), N,N-ditetradecylamine-N-succinyl[methyl(polysarcosine)35](N-Tetamine-pSar35), N,N-ditetradecylamine-N-succinyl[methyl(polysarcosine)45](N-Tetamine-pSar45), N-tetradecyl polysarcosine-25, N-Hexadecyl polysarcosine-25, and the like.

In other embodiments, the pegylated lipid is a PEG-ceramide (e.g., C8 PEG750 ceramide (N-octanoyl-sphingosine-1-{succinyl[methoxy(polyethylene glycol)750]}), C16 PEG750 ceramide (N-palmitoyl-sphingosine-1-{succinyl[methoxy(polyethylene glycol) 750]}), C8 PEG2000 ceramide (N-octanoyl-sphingosine-1-{succinyl[methoxy(polyethylene glycol)2000]}), C16 PEG2000 ceramide (N-palmitoyl-sphingosine-1-{succinyl[methoxy (polyethylene glycol)2000]}), C8 PEG5000 ceramide (N-octanoyl-sphingosine-1-{succinyl [methoxy(polyethylene glycol)5000]}), C16 PEG5000 ceramide (N-palmitoyl-sphingosine-1-{succinyl[methoxy(polyethylene glycol)5000]}), and the like).

In another instance, the amphiphile can be an anionic lipid or a cationic lipid. Non-limiting anionic lipid include 1,2-di-O-tetradecyl-sn-glycero-3-phospho-(1′-rac-glycerol), 1,2-dihexadecanoyl-sn-glycero-3-phospho-(1′-sn-glycerol)). Non-limiting cationic lipids include DC-cholesterol (30-[N (N′,N′-dimethylaminoethane)-carbamoyl]cholesterol), 18:1 TAP (DOTAP) (1,2-dioleoyl-3-trimethylammonium-propane), 1-oleoyl-2-[6-[(7-nitro-2-1,3-benzoxadiazol-4-yl)amino]hexanoyl]-3-trimethylammonium propane, 14:0 TAP (1,2-dimyristoyl-3-trimethylammonium-propane), 16:0 TAP (1,2-dipalmitoyl-3-trimethyl ammonium-propane), 18:0 TAP (1,2-stearoyl-3-trimethylammonium-propane), DOTMA (1,2-di-O-octadecenyl-3-trimethylammonium propane), a phosphatidylcholine (e.g., 12:0 EPC (1,2-dilauroyl-sn-glycero-3-ethylphosphocholine), 14:0 EPC (1,2-dimyristoyl-sn-glycero-3-ethylphosphocholine), 14:1 EPC (1,2-dimyristoleoyl-sn-glycero-3-ethyl phosphocholine), 16:0 EPC (1,2-dipalmitoyl-sn-glycero-3-ethylphosphocholine), 18:0 EPC (1,2-distearoyl-sn-glycero-3-ethylphosphocholine), 18:1 EPC (1,2-dioleoyl-sn-glycero-3-ethylphosphocholine), 16:0-18:1 EPC (1-palmitoyl-2-oleoyl-sn-glycero-3-ethylphosphocholine), 6-((2-hexyldecanoyl)oxy)-N-(6-((2-hexyldecanoyl)oxy)hexyl)-N-(4-hydroxybutyl)hexan-1-aminium (ALC-0315), 2-[(polyethylene glycol)-2000]-N,N-ditetradecylacetamide (ALC-0159), (6Z,9Z,28Z,31Z)-heptatriacont-6,9,28,31-tetraene-19-yl 4-(dimethylamino)butanoate (DLin-MC3-DMA or MC3), 2-[2,2-bis[(9Z,12Z)-octadeca-9,12-dienyl]-1,3-dioxolan-4-yl]-N,N-dimethylethanamine (DLin-KC2-DMA), heptadecan-9-yl 8-((2-hydroxyethyl)(6-oxo-6-(undecyloxy)hexyl)amino)octanoate (SM-102), A18-Iso5-2DC218 (see, e.g., Miao et al., Nat. Biotech. 37, 1174-1185 (2019)), A6 (see, e.g., Miao et al., Nat. Commun. 11, 2424 (2020), 306Oᵢ₁ₒ (see, e.g., Hajj et al., Nano Lett. 20, 7, 5167-5175 (2020)), 2,3-dioleyloxy-N-[2-(sperminecarboxamido)ethyl]-N,N-dimethyl-1-propanaminium (DOSPA, including salts thereof, such as HCl salt, a trifluoroacetate salt, chloride salt, etc.), and the like. In particular embodiments, any of these lipids can be considered an ionizable lipid and/or a cationic ionizable lipid. Other lipids are described in Miao et al., Nat. Biotech. 37, 1174-1185 (2019).

Yet other cationic lipids may be, e.g., 1,2-dilinoleyloxy-N,N-dimethylaminopropane (DLinDMA), 1,2-dilinolenyloxy-N,N-dimethylaminopropane (DLenDMA), N,N-dioleyl-N,N-dimethylammonium chloride (DODAC), dioctadecyldimethylammonium (DODMA), distearyldimethylammonium (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N-(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′, 1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-dilinoleylcarbamyl-3-dimethylaminopropane (DLincarbDAP), 1,2-dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), or mixtures thereof. Cationic lipids such as CLinDMA or DLin-K-DMA, as well as additional cationic lipids described in, e.g., U.S. Pat. Pub. Nos. 2006/0240554 and 2009/0291131.

Further compounds (e.g., amphiphiles, lipids, polymers, and the like) are described in Int. Pub. Nos. WO 2016/065306 and WO 2018/089688, each of which is incorporated herein by reference in its entirety.

### Polymers

The compositions herein can include one or more polymers (e.g., one or more water-insoluble polymers), in which polymers can include homopolymers, heteropolymers, or copolymers. The polymer(s) can be present within the core or can even be absent. In certain instances, polymer(s) can interact with the outer layer disposed around the core or be present within the core. In yet other instances, the polymer can be modified to include one or more functional moieties (e.g., such as polymers modified with one or more PEGs, lipids, or combinations thereof). In particular embodiments, the polymer can include an amphiphile (e.g., an amphiphilic polymer). In other embodiments, the polymer can include a hydrophobic polymer.

Polymer can include any molecular structure including one or more repeat units (monomers), connected by covalent bonds. The polymer may be a copolymer, in which the molecular structure includes two or more monomers. The repeat units forming the copolymer may be arranged in any fashion. For example, the repeat units may be arranged in a random order, in an alternating order, or as a “block” copolymer, i.e., including one or more regions each including a first repeat unit (e.g., a first block), and one or more regions each including a second repeat unit (e.g., a second block), etc. Block copolymers may have two (a diblock copolymer), three (a triblock copolymer), or more numbers of distinct blocks.

Any useful polymer can be employed. For instance, polymers (including copolymers) can include, but are not limited to, polymers including glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA,” and caprolactone units, such as poly(8-caprolactone), collectively referred to herein as “PCL”; copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; polyacrylates; polyanhydrides; polyester anhydrides; polyhydroxybutyrates, including 4-hydroxybutyrate (P4HB); as well as combinations and derivatives thereof.

In some embodiments, the polymer is a water-insoluble polymer. Non-limiting water-insoluble polymers can include homopolymers (i.e., synthesized from hydrophobic monomers (e.g., styrene, methyl methacrylate, glycidyl methacrylate, DL-lactide, and the like)), random copolymers (i.e., synthesized from two or more monomers (e.g., styrene, methyl methacrylate, glycidyl methacrylate, DL-lactide, acrylic acid, methacrylic acid, 2-hydroxyethyl acrylate, and the like)), block polymers (i.e., synthesized from two or more monomers (e.g., styrene, methyl methacrylate, glycidyl methacrylate, DL-lactide, acrylic acid, methacrylic acid, 2-hydroxyethyl acrylate, and the like)), graft polymers (e.g., synthesized from artificial polymers (polyacrylic acid, polyglycidyl methacrylate, and the like), and/or natural polymers (e.g., dextran, starch, chitosan, and the like) with functional pendent groups (e.g., amino, carboxylate, hydroxyl, epoxy groups, and the like)), and/or branched polymers (e.g., a hyperbranched polyester with multifunctional alcohol building block and 2,2-bis(methylol)propionic acid branching units, such as Boltorn™ H40).

Yet other non-limiting polymers can include, e.g., poly(glycolic acid), poly(lactic acid), poly(caprolactone), poly(lactide-co-glycolide), poly(ortho ester) II, poly(alkyl cyanoacrylate), desaminotyrosyl octyl ester, polyphosphoesters, polyester amides, polyurethanes, and lipids. Other non-limiting examples of polymers that the core can comprise include: chitosan; acrylates copolymer; acrylic acid-isooctyl acrylate copolymer; ammonio methacrylate copolymer; ammonio methacrylate copolymer type A; ammonio methacrylate copolymer type B; butyl ester of vinyl methyl ether/maleic anhydride copolymer (125,000 molecular weight); carbomer homopolymer type A (allyl pentaerythritol crosslinked); carbomer homopolymer type B (allyl sucrose crosslinked); cellulosic polymers; dimethylaminoethyl methacrylate-butyl methacrylate-methyl methacrylate copolymer; dimethylsiloxane/methylvinylsiloxane copolymer; divinylbenzene styrene copolymer; ethyl acrylate-methacrylic acid copolymer; ethyl acrylate and methyl methacrylate copolymer (2:1; 750,000 molecular weight); ethylene vinyl acetate copolymer; ethylene-propylene copolymer; ethylene-vinyl acetate copolymer (28% vinyl acetate); glycerin polymer solution i-137; glycerin polymer solution IM-137; hydrogel polymer; ink/polyethylene terephthalate/aluminum/polyethylene/sodium polymethacrylate/ethylene vinyl acetate copolymer; isooctyl acrylate/acrylamide/vinyl acetate copolymer; Kollidon® VA 64 polymer; methacrylic acid-ethyl acrylate copolymer (1:1) type A; methacrylic acid-methyl methacrylate copolymer (1:1); methacrylic acid-methyl methacrylate copolymer (1:2); methacrylic acid copolymer; methacrylic acid copolymer type A; methacrylic acid copolymer type B; methacrylic acid copolymer type C; octadecene-1/maleic acid copolymer; PEG-22 methyl ether/dodecyl glycol copolymer; PEG-45/dodecyl glycol copolymer; polyester polyamine copolymer; poly(ethylene glycol) (PEG) 1,000; PEG 1,450; PEG 1,500; PEG 1,540; PEG 200; PEG 20,000; PEG 200,000; PEG 2,000,000; PEG 300; PEG 300-1,600; PEG 300-1,600; PEG 3,350; PEG 3,500; PEG 400; PEG 4,000; PEG 4,500; PEG 540; PEG 600; PEG 6,000; PEG 7,000; PEG 7,000,000; PEG 800; PEG 8,000; PEG 900; polyvinyl chloride-polyvinyl acetate copolymer; povidone acrylate copolymer; povidone/eicosene copolymer; polyoxy(methyl-1,2-ethanediyl), alpha-hydro-omega-hydroxy-, polymer with 1,1′-methylenebis[4-isocyanatocyclohexane]copolymer; polyvinyl methyl ether/maleic acid copolymer; styrene/isoprene/styrene block copolymer; vinyl acetate-crotonic acid copolymer; {poly[(9,9-di-n-octylfluorenyl-2,7-diyl)-alt-(benzo[2,1,3]thiadiazol-4,8-diyl)]}, and {poly[2,6-(4,4-bis-(2-ethylhexyl)-4H-cyclopenta [2,1-b;3,4-b′]dithiophene)-alt-4,7(2,1,3-benzothiadiazole)]}.

In some embodiments, the polymer can include a hydrophobic polymer. Non-limiting examples of hydrophobic polymers include, but are not limited to: polylactic acid (PLA), polypropylene oxide, poly(lactide-co-glycolide) (PLGA), poly(epsilon-caprolactone), poly(ethylethylene), polybutadiene, polyglycolide, polymethacrylate, polyvinylbutylether, polystyrene, polycyclopentadienylmethylnorbornene, polyethylenepropylene, polyethylethylene, polyisobutylene, polysiloxane, a polymer of any of the following: methyl acrylate, ethyl acrylate, propyl acrylate, n-butyl acrylate, isobutyl acrylate, 2-ethyl acrylate, t-butyl acrylate, methacrylates (e.g., ethyl methacrylate, n-butyl methacrylate, and isobutyl methacrylate), acrylonitriles, methacrylonitrile, vinyls (e.g., vinyl acetate, vinylversatate, vinylpropionate, vinylformamide, vinylacetamide, vinylpyridines, and vinyllimidazole), aminoalkyls (e.g., aminoalkylacrylates, aminoalkylmethacrylates, aminoalkyl(meth)acrylamides), styrenes, and lactic acids.

In certain embodiments, the polymer can include amphiphilic polymers or amphiphilic copolymers. Without wishing to be limited by mechanism, amphiphilic polymers can spontaneously self-assemble in aqueous solution to form NPs with a hydrophobic inner core and a hydrophilic outer layer or shell. The hydrophobic inner core can be used to deliver therapeutic and diagnostic agents including genes, proteins, chemotherapeutic drugs, or other small molecules. In some embodiments, the amphiphilic polymer can include a hydrophilic segment (e.g., any described herein, such as those present in a hydrophilic polymer) and a hydrophobic segment (e.g., any described herein, such as those present in a hydrophobic polymer). In some instances, the hydrophilic segment of the amphiphilic polymer can be configured to orient to the exterior of the nanoparticles when formed by emulsion techniques such as self-assembly.

In some embodiments, the delivery vehicles includes amphiphile-polymer particles, e.g., including a water-insoluble polymeric core, a payload, and at least one amphiphile within the core, as described in Int. Pub. Nos. WO 2016/065306 and WO 2018/089688, each of which is incorporated herein by reference in its entirety.

In some embodiments, the delivery vehicles can include a polymeric matrix, wherein the polymeric matrix comprises a lipid-terminated polymer such as polyalkylene glycol and/or a polyester. In some embodiments, the delivery vehicle includes an amphiphilic lipid-terminated polymer, where a cationic and/or an anionic lipid is conjugated to a hydrophobic polymer. In one embodiment, the polymeric matrix comprises lipid-terminated PEG. In other embodiments, the polymeric matrix includes lipid-terminated copolymer.

In other embodiments, the polymeric matrix comprises lipid-terminated PEG and PLGA. In one embodiment, the lipid is 1,2 distearoyl-sn-glycero-3-phosphoethanolamine (DSPE), and salts thereof. In another embodiment, the polymeric matrix comprises DSPE-terminated PEG. The lipid-terminated PEG can then, for example, be mixed with PLGA to form a nanoparticle.

In yet other embodiments, the compositions can include a core of mRNA complexed with a cationic lipid-like compound (e.g., any Gₘ-Cₙ compound herein) and a poly(lactic-co-glycolic acid) (PLGA) polymer, in which the core is surrounded by an outer lipid layer including a lipid-poly(ethylene glycol) (lipid-PEG), e.g., (e.g., DSPE-PEG (1,2-distearoyl-sn-glycero-3-phosphoethanolamine-N-[methoxy{polyethylene glycol}]) or ceramide-PEG (N-palmitoyl-sphingosine-1-(succinyl{methoxy[polyethylene glycol]}) with PEG molecular weight (MW) 2000-5000 (see, e.g., FIG. 1a herein). Gₘ-Cₙ can be used for mRNA complexation, and PLGA, a widely clinically used biodegradable and biocompatible polymer, can provide a stable NP core. In particular embodiments, a biocompatible polymer is used here to refer to a polymer that does not typically induce an adverse response when inserted or injected into a living subject, for example, without significant inflammation and/or acute rejection of the polymer by the immune system, for instance, via a T-cell response.

Besides amphiphilic polymers and amphiphilic copolymers, hydrophobic polymers can be also used to develop stimuli-responsive NPs for various biomedical applications. For these hydrophobic polymers, their NPs can be prepared by using the mixture of the hydrophobic polymer and amphiphilic polymer or amphiphilic compound. The amphiphilic compound can include, but is not limited to, one or a plurality of naturally derived lipids, lipid-like materials, surfactants, or synthesized amphiphilic compounds.

Optionally, the incorporation of stimuli-responsive moieties to the hydrophobic core can accomplish the spatiotemporal control over the macroscopic properties of NPs, and thereby the release of the encapsulated cargo at the desired site. The amphiphilic polymers can be responsive to a stimulus. This may be a pH change, redox change, temperature change, exposure to light, or other stimuli, including binding to a target. The responsiveness may be imparted solely by the hydrophilic polymer, the hydrophobic polymer or the conjugate per se. The nanoparticles can be formed of a mixture or blend of polymers. Some may be the amphiphilic polymers, e.g., such as copolymers of modified polyethylene glycol (PEG) and polyesters, including various forms of PLGA-PEG or PLA-PEG copolymers, collectively referred to herein as “PEGylated polymers”, some hydrophobic polymer such as PLGA, PLA or PGA, and/or some may be hydrophilic polymer such as a PEG or PEG derivative. Some can be modified by conjugation to a targeting agent, a targeting moiety, a targeting ligand, a cell adhesion moiety, a cell penetrating peptide, as well as other targeting compounds described herein.

In some embodiments, long-circulating, optionally cell-penetrating, and stimuli-responsive nanoparticles for effective in vivo delivery of therapeutic, prophylactic and/or diagnostic agents are used. In one embodiment, the NPs are made of an amphiphilic polymer, e.g., a pegylated polymer (i.e., a polymer having one or more PEG groups), which shows a response to a stimulus such as pH, temperature, or light, such as an ultra pH-responsive characteristic with a pKa close to the endosomal pH (6.0-6.5) (Wang Y et al., Nat. Mater. 13, 204-212 (2014)). The polymer may include a targeting or cell penetrating or adhesion molecule such as a tumor-penetrating peptide iRGD.

Stimuli responsive polymers and stimuli responsive amphiphilic polymers can be formed through selection of a hydrophilic or hydrophobic polymer components of the polymer, or by modification of the hydrophilic or hydrophobic polymers.

In some embodiments, the nanoparticles can be formed by self-assembly in an emulsion of a non-aqueous solvent with an aqueous solvent of a first amphiphilic polymer containing a polymer represented by Formula I.

(X)ₘ—(Y)ₙ, Formula I
wherein each of m and n is, independently, an integer between one and 1000, inclusive; X is a hydrophobic polymer; Y is a hydrophilic polymer; and at least one of X, Y, or both, is stimuli-responsive.

Optionally, the nanoparticles are formed by self-assembly of a mixture of polymers represented by Formula I and a second polymer containing a polymer represented by Formula II:

(Q)_{c}—(R)_{d}, Formula II

wherein each of c and d is, independently, an integer between zero and 1000, inclusive, with the proviso that the sum (c+d) is greater than one; and each of Q and R is, independently, a hydrophilic or hydrophobic polymer. Optionally, the nanoparticles are formed by self-assembly of a mixture of polymers represented by Formula I and Formula II, wherein the polymer represented by Formula I, Formula II, or both, contains a ligand, wherein the ligand is a targeting ligand, an adhesion ligand, a cell-penetrating ligand, or an endosomal-penetrating ligand, with the proviso that the ligand is conjugated to the hydrophilic polymer.

In some embodiments, the nanoparticles are formed by self-assembly of a mixture of first stimuli-responsive hydrophobic polymer and a second polymer containing a polymer represented by Formula III:

(S)ₑ-(T)_{f}, Formula III
wherein each of e and f is, independently, an integer between zero and 1000, inclusive, with the proviso that the sum (e+f) is greater than one; and each of S and T is, independently, a hydrophilic polymer or a hydrophobic polymer. Optionally, the first stimuli-response hydrophobic polymer, the polymer represented by Formula III, or both contains a ligand, wherein the ligand is a targeting ligand, an adhesion ligand, a cell-penetrating ligand, or an endosomal-penetrating ligand, with the proviso that the ligand is conjugated to the hydrophilic polymer.

In some embodiments, the nanoparticles are formed by self-assembly of a mixture of first stimuli-responsive hydrophilic polymer and a second polymer containing a polymer represented by Formula III:

(S)ₑ-(T)_{f}, Formula III
wherein each of e and f is, independently, an integer between zero and 1000, inclusive, with the proviso that the sum (e+f) is greater than one; and each of S and T is, independently, a hydrophilic polymer or a hydrophobic polymer. Optionally, the first stimuli-response hydrophilic polymer, the polymer represented by Formula III, or both contains a ligand, wherein the ligand is a targeting ligand, an adhesion ligand, a cell-penetrating ligand, or an endosomal-penetrating ligand, with the proviso that the ligand is conjugated to the hydrophilic polymer.

Optionally, the polymers that form the nanoparticles contain linkers between the blocks of hydrophilic and hydrophobic polymers, between the hydrophilic polymer and ligand, or both.

In some embodiments, the polymer is a biocompatible polymer. One non-limiting test to determine biocompatibility is to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells such as fibroblasts or epithelial cells, even if phagocytosed or otherwise taken up by such cells.

The biocompatible polymer can be biodegradable, e.g., the polymer is able to degrade, chemically and/or biologically, within a physiological environment, such as within the body.

The polymers can be responsive to changes in pH-, redox-, light-, temperature-, enzyme-, ultrasound, or other stimuli such as a conformation change resulting from binding.

Almeida et al., J. Applied Pharm. Sci. 2(6), 1-10 (2012) provides a review of stimuli responsive polymers. The signs or stimuli that trigger the structural changes on smart polymers can be classified in three main groups: physical stimuli (e.g., temperature, ultrasound, light, or mechanical stress), chemical stimuli (e.g., pH or ionic strength), and biological stimuli (e.g., enzymes or biomolecules).

Stimuli can be artificially controlled (with a magnetic or electric field, light, ultrasounds, etc.) or naturally promoted by internal physiological environment through a feedback mechanism, leading to changes in the polymeric network. In turn, such changes can allow for delivery of the cargo without any external intervention (for example, pH changes in certain vital organs or related to a disease; temperature change or presence of enzymes or other antigens) or by the physiological condition. In the presence of a sign or stimuli, changes can occur on the surface and solubility of the polymer as well as on sol-gel transition.

Smart polymers can be classified according to the stimuli they respond to or to their physical features. Regarding the physical shape, they can be classified as free linear polymer chain solutions, reversible gels covalently cross-linked and polymer chain grafted to the surface.

Stimuli responsive polymers are also reviewed by James et al., Acta Pharma. Sinica B 4(2), 120-127 (2014). The following is a list of exemplary polymers categorized by responsive to various stimuli: temperature, such as for poloxamers, poly(N-alkylacrylamide)s, poly(N-vinylcaprolactam)s, cellulose, xyoglucan, and chitosan; pH, such as for poly(methacrylic acid)s, poly(vinylpyridine)s, and poly(vinylimidazole)s; light, such as for modified poly(acrylamide)s; electric field, such as for sulfonated polystyrenes, poly(thiophene)s, and poly(ethyloxazoline)s; and ultrasound, such as for ethylenevinylacetate.

These transitions can be reversible and can include changes in physical state, shape and solubility, solvent interactions, hydrophilic and lipophilic balances, and conductivity. The driving forces behind these transitions can include neutralization of charged groups by the addition of oppositely charged polymers or by pH shift, as well as changes in the hydrophilic/lipophilic balance or changes in hydrogen bonding due to increase or decrease in temperature. Responses of a stimulus-responsive polymer can be of various types. Responsiveness of a polymeric solution initiated by physical or chemical stimuli can include destruction and formation of various secondary forces, including hydrogen bonding, hydrophobic forces, van der Waals forces, and electrostatic interaction. Chemical events can include simple reactions, such as oxidation, acid-base reaction, reduction, and hydrolysis of moieties attached to the polymer chain. In some cases, dramatic conformational change in the polymeric structure can occur, e.g., degradation of the polymeric structure due to irreversible bond breakage in response to an external stimulus.

Exemplary pH dependent polymers include dendrimers formed of poly(lysine), poly(hydroxyproline), PEG-PLA, poly(propyl acrylic acid), poly(ethacrylic acid), Carbopol®, polysilamine, Eudragit® 5-100, Eudragit® L-100, chitosan, PMAA-PEG copolymer, sodium alginate (Ca²⁺). The ionic pH sensitive polymers are able to accept or release protons in response to pH changes. These polymers contain acid groups (carboxylic or sulfonic) or basic groups (ammonium salts), so that the pH sensitive polymers are polyelectrolytes that have in their structure acid or basic groups that can accept or release protons in response to pH changes in the surrounding environment. pH values from several tissues and cell compartments can be used to trigger release in these tissues. For example, the pH of blood is 7.4-7.5; stomach is 1.0-3.0; duodenum is 4.8-8.2; colon is 7.0-7.5; lysosome is 4.5-5.0; Golgi complex is 6.4; and tumor—extracellular medium is 6.2-7.2.

Examples of these polymers include poly(acrylic acid) (PAA) (Carbopol®) and derivatives, poly(methacrylic acid) (PMAA), poly(2-(diisopropylamino) ethylmethacrylate) (PDPA), poly(2-(hexamethyleneimino) ethyl methacrylate), poly(2-diethylaminoethyl methacrylate) (PDEAEMA), poly(ethylene imine), poly(L-lysine), and poly(N,N-dimethylaminoethylmethacrylate) (PDMAEMA). Polymers with functional acid groups (e.g., pH sensitive polymers) include poly(acrylic acid) (PAA) or poly(methacrylic) acid (PMAA), which are polyanions having ionizable acid groups (e.g., carboxylic acid or sulfonic acid). The pH in which acids become ionized can depend on the pKa of the polymer, as well as the composition and molecular weight of the polymer. Polymers with functional basic groups include polycations, such as poly(4-vinylpyridine), poly(2-vinylpyridine) (PVP) and poly(vinylamine) (PVAm), which are protonated at high pH values and positively ionized at neutral or low pH values. In particular, these polymer have a phase transition at pH 5 due to the deprotonation of the pyridine groups. Other polybases are poly(N,N-dimethylaminoethyl methacrylate) (PDMAEMA) and poly(2-diethylaminoethyl methacrylate) (PDEAEMA) with amino groups in their structure, which can gain protons is acidic environments and release protons in basic environments. Examples of polycationic polyelectrolyte polymers are poly(N,N-dialkyl aminoethyl methacrylate), poly(lysine) (PL), poly(ethylenimine) (PEI), and chitosan. Commercially available polymers include Eudragit L® and Eudragit S® from Rohm Pharma GmBH (with methacrylic acid and methylmethacrylate in their composition), CMEM (a cellulose derivative) from Freund Sangyo Co., CAP by Wako Pure Chemicals Ltd., as well as HP-50 and ASM by Shin-Etsu Chemical Co., Ltd.

The length of hydrophilic and/or hydrophobic polymers can be optimized to optimize encapsulation of agent to be delivered, e.g., as determined by encapsulation efficiency (EE %). In some embodiments, EE % refers to a fraction of initial drug that is encapsulated by a delivery vehicle (e.g., a nanoparticle). In non-limiting embodiments, as the PDPA length increases, the EE % and size of the resulting NPs can increase, possibly because the increased PDPA length leads to an increase in the size of the hydrophobic core. Specifically, the EE % reaches almost 100% for the polymer with 80 (PDPA80) or 100 (PDPA100) DPA repeat units. Notably, using a mixture of Meo-PEG-b-P(DPA-co-GMA-TEPA-C14) (90 mol %) and tumor-penetrating polymer (iRGD-PEG-b-PDPA, 10 mol %) to prepare NPs does not cause obvious change in the EE % or particle size.

There are several natural polymers (for example, albumin, gelatin and chitosan) that present pH sensibility. Chitosan is a cationic amino polysaccharide, derivative from chitin, which is biocompatible and resorbable. Additional examples include the anionic polymer PEAA (polyethacrylic acid), PPAA (polypropyl acrylic acid), copolymer of polypropylacrylic acid (PPAA) and polyethacrylic acid (PEAA), poly(ethylene glycol)-poly(aspartame hydrazine doxorubicin) [(PEG-p(Asp-Hid-dox), and polycationic polymers, such as poly(2-diethylaminoethyl methacrylate) (PDEAEMA).

Temperature-dependent polymers are sensitive to the temperature and change their microstructural features in response to change in temperature. Thermo-responsive polymers present in their structure a very sensitive balance between the hydrophobic and the hydrophilic groups, and a small change in the temperature can create new adjustments. If the polymeric solution has a phase below the critical solution temperature, it will become insoluble after heating. Above the lower critical solution temperature (LCST), the interaction strength (hydrogen linkages) between the water molecules and the polymer becomes unfavorable. In response, the polymer dehydrates; and a predominance of the hydrophobic interaction occurs, causing the polymer to swell. The LCST is the critical temperature in which the polymeric solution shows a phase separation, going from one phase (isotropic state) to two phases (anisotropic state). The accumulation of temperature-sensitive polymeric systems in solid tumors can be due to the increased impermeability effect to the tumor vascular net retention and to the use of an external impulse (heat source) on the tumor area. This temperature increase can promote the changing of the microstructure of the polymeric system, turning it into gel and releasing the drug, thus increasing the drug in the intratumoral area and the therapeutic efficiency, and reducing the side effects.

Examples of thermosensitive polymers include the poly(N-substituted acrylamide) polymers such as poly(N-isopropylacrylamide) (PNIPAAm), poly (N,N′-diethylacrylamide), poly (dimethylaminoethylmethacrylate and poly (N-(L)-(1-hydroxymethyl)propyl methacrylamide). Other examples of thermo-responsive polymers are: copolymers blocks of poly(ethylene glycol)/poly(lactide-co-glycolide) (PEG/PLGA, REGEL®), polyoxyethylene/polyoxypropylene (PEO/PPO), triple blocks of copolymers polyoxyethylene-polyoxypropylene-polyoxyethylene (PEO-PPO-PEO) and poly(ethylene glycol)-poly(lactic acid)-poly(ethylene glycol) (PEG-PLA-PEG), and the like. Exemplary polymers and their LCST include the following: PNIPAAm, LCST 32° C.; PDEAAm, LCST 26-35° C.; PDMAEMA, LCST 50° C.; and poly(N-(L)-(hydroxymethyl)propylmethacrylamide), LCST 30° C.

An increase of the hydrophobic monomers (as, for example, the butyl methacrylate) or on the molecular weight, generally results in a decrease in LCST. The incorporation of hydrophilic monomers (e.g., acrylic acid or hydroxyethyl methacrylate) fosters the creation of increases LCST. The co-polymers NIPAAm conjugated with hydrophilic moieties (e.g., such as acrylic acid) can promote the increase of LCST to temperatures around 37° C., i.e., the body temperature. In another example, polymers with 2-hydroxyethyl (methacrylate) (HEMA) promote the increase of LCST above the body temperature

Poloxamers and derivatives are well known temperature sensitive polymers. The copolymer blocks based on PEO-PPO sequences constitutes one family of triple blocks of commercialized copolymers with the following names: Pluronics®, Poloxamers®, and Tetronics®. Poloxamers® are non-ionic polymers of polyoxyethylene-polyoxypropylene-polyoxyethylene (PEOn-PPOn-PEOn), with many pharmaceutical uses. The triple block (or triblock) of copolymers PEO-PPO-PEO (Pluronics® or Poloxamers®) can form a gel at body temperature in concentrations above 15% (m/m). Non-limiting Poloxamers® can include, e.g., 188 (F-68), 237 (F-87), 338 (F-108) and 407 (F-127). “F” refers to the polymer in the form of flakes. Pluronics® and Tetronics® are polymers approved by FDA to be used as food additives, pharmaceutical ingredients, drug carriers in parenteral systems, tissue engineering materials, and agricultural products. Pluronic F-127 (Poloxamer 407, PF-127) can also be used as carrier in several routes of administration, including oral, cutaneous, intranasal, vaginal, rectal, ocular, and parenteral. Pluronic® F127 (PF-127) or Poloxamer 407 (P407) (copolymer polyoxyethylene 106-polyoxypropylene 70-polyoxyethylene 106) contains about 70% of ethylene oxide, which contributes to its hydrophilicity.

To obtain a temperature and pH sensitive polymer, temperature-sensitive monomers (as, for example, poly(N-isopropylacrylamide-co-methacrylic acid and PNIPAAm) with pH sensitive monomers (as, for example, acrylic acid (AA) and methacrylic acid (MAA)) can be combined.

Biologically responsive polymer systems can be useful in various biomedical applications. One advantage of bioresponsive polymers is that they can respond to the stimuli that are inherently present in the natural system. Bioresponsive polymeric systems mainly arise from common functional groups that are known to interact with biologically relevant species, and in other instances the synthetic polymer is conjugated to a biological component. Bioresponsive polymers are classified into antigen-responsive polymers, glucose-sensitive polymers, and enzyme-responsive polymers.

Glucose-responsive polymeric-based systems have been developed based on the following approaches: enzymatic oxidation of glucose by glucose oxidase (GOx), and binding of glucose with lectin or reversible covalent bond formation with phenylboronic acid moieties. Glucose sensitivity occurs by the response of the polymer toward the byproducts that result from the enzymatic oxidation of glucose. Glucose oxidase oxidizes glucose resulting in the formation of gluconic acid and H₂O². For example, in the case of poly(acrylic acid) (PAA) conjugated with the GOx system, as the blood glucose level is increased glucose is converted into gluconic acid which causes the reduction of pH and protonation of PAA carboxylate moieties, facilitating the release of insulin.

Another system uses the carbohydrate binding properties of lectin for the fabrication of a glucose-sensitive system. Concanavalin A (Con A) is a lectin possessing four binding sites and has been used frequently in insulin-modulated drug delivery. In this type of system, the insulin moiety is chemically modified by introducing a functional group (or glucose molecule) and then attached to a carrier or support through specific interactions which can only be interrupted by the glucose itself. The glycosylated insulin-Con A complex exploits the competitive binding behavior of Con A with glucose and glycosylated insulin. The free glucose molecule causes the displacement of glycosylated Con A-insulin conjugates.

Another approach includes polymers with phenylboronic groups and polyol polymers that form a gel through complex formation between the pendant phenylborate and hydroxyl groups. Instead of polyol polymers, short molecules such as diglucosylhexadiamine have been used. As the glucose concentration increases, the crosslinking density of the gel decreases and as a result insulin is released from the eroded gel. The glucose exchange reaction is reversible and reformation of the gel occurs as a result of borate-polyol crosslinking.

Field-responsive polymers respond to the application of electric, magnetic, sonic, ultrasonic, or electromagnetic fields. The additional benefit over traditional stimuli-sensitive polymers is their fast response time, anisotropic deformation due to directional stimuli, and also a controlled drug release rate simply by modulating the point of signal control.

A light-sensitive polymer undergoes a phase transition in response to exposure to light. These polymers can be classified into UV-sensitive and visible-sensitive systems on the basis of the wavelength of light that triggers the phase transition. A variety of materials are known, such as a leuco-derivative molecule, bis(4-dimethylamino)phenylmethyl leucocyanide, which undergoes phase transition behavior in response to UV light. Triphenylmethane-leuco derivatives dissociate into ion pairs, e.g., such as triphenylmethyl cations, upon UV irradiation. At a fixed temperature, these hydrogels swell discontinuously due to increased osmotic pressure in response to UV irradiation but shrink when the stimulus is removed. Another example is a thermosensitive diarylated Pluronic® F-127.

Visible light-sensitive polymeric materials can be prepared by incorporating photosensitive molecules, such as chromophores (e.g., trisodium salt of copper chlorophyllin). When light of appropriate wavelength is applied, the chromophore absorbs light which is then dissipated locally as heat by radiationless transition, increasing the local temperature of the polymeric material, leading to alteration of the swelling behavior. The temperature increase directly depends on the chromophore concentration and light intensity.

Electric field-sensitive polymers change their physical properties in response to a small change in electric current. These polymers contain a relatively large concentration of ionizable groups along the back bone chain that are also pH-responsive. Electro-responsive polymers transform electric energy into mechanical energy. The electric current can cause a change in pH, which leads to disruption of hydrogen bonding between polymer chains, thereby causing degradation or bending of the polymer chain. Major mechanisms involved in drug release from electro-responsive polymer are diffusion, electrophoresis of charged drug, forced convection of drug out of the polymer, or degradation of the polymer.

Naturally-occurring polymers (e.g., such as chitosan, alginate, and hyaluronic acid) can be employed to prepare electro-responsive materials. Major synthetic polymers that have been used include allyl amine, vinyl alcohol, acrylonitrile, methacrylic acid and vinylacrylic acid. In some cases, combinations of natural and synthetic polymers have been used. Most polymers that exhibit electro-sensitive behavior are polyelectrolytes and undergo deformation under an electric field due to anisotropic swelling or deswelling as the charged ions move towards the cathode or anode. Neutral polymers that exhibit electro-sensitive behavior can includes a polarizable component with the ability to respond to the electric field. Another example of a material that can be used is poly(2-acrylamido-2-methylpropane sulphonic acid-co-n-butylmethacrylate).

In some embodiments, the delivery vehicles for the nucleic acids are formed from a biocompatible, hydrogel-forming polymer encapsulating the nucleic acids to be delivered. In some embodiments, the hydrogel is an anionic polymer that is cross-linked with a polycationic polymer. In some embodiments, the nanoparticles are configured with a core and envelope structure. In these embodiments, the nucleic acids can be encapsulated in the core hydrogel, and the drug-loaded polymeric particles are encapsulated within the envelope hydrogel. In some embodiments, the core and envelope hydrogels are separated by a membrane, layer, or shell.

Examples of materials that can be used to form a suitable hydrogel include polysaccharides such as alginate, polyphosphazines, poly(acrylic acids), poly(methacrylic acids), poly(alkylene oxides), poly(vinyl acetate), polyvinylpyrrolidone (PVP), and copolymers and blends of each. See, for example, U.S. Pat. Nos. 5,709,854, 6,129,761 and 6,858,229, each of which is incorporated herein by reference in its entirety.

In general, these polymers are at least partially soluble in aqueous solutions, such as water, buffered salt solutions, or aqueous alcohol solutions. In some embodiments, the polymer can have one or more charged side groups or a monovalent ionic salt thereof. Examples of polymers with acidic side groups that can be reacted with cations are poly(phosphazenes), poly(acrylic acids), poly(methacrylic acids), poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups and sulfonic acid groups, as well as deprotonated forms thereof.

Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups, as well as protonated forms thereof.

The biocompatible, hydrogel-forming polymer can be a water-soluble gelling agent. In some embodiments, the water-soluble gelling agent is a polysaccharide gum or a polyanionic polymer.

In some embodiments, the targeting ligands are covalently attached to hydrogel-forming polymers. In some embodiments, the nucleic acids to be targeted are attached to the hydrogel forming polymer via a linking moiety that is designed to be cleaved in vivo. The composition of the linking moiety can also be selected in view of the desired release rate of the nucleic acids.

Further polymers (e.g., including hydrophilic, hydrophobic, water-insoluble, or amphipathic polymers) and other compounds (e.g., amphiphiles, lipids, and the like) are described in Int. Pub. Nos. WO 2016/065306 and WO 2018/089688, each of which is incorporated herein by reference in its entirety.

### Lipid-Like Compounds, Including Ionizable Lipid-Like Compounds

The lipid-like compound can include any compound having a lipophilic moiety that is useful for complexing a cargo. Non-limiting lipid-like compounds include, e.g., a polymer (e.g., including an amphiphilic polymer or copolymer) having a lipophilic moiety. Further examples of lipid-like compounds include, e.g., a poly(amidoamine) (PAMAM) dendrimer (e.g., generation 0, 1, 2, 3, 4, 5, or more), such as a Gₘ-Cₙ compound; a polypropyleneimine tetramine dendrimer (e.g., generation 0, 1, 2, 3, 4, 5, or more); and a ethylenediamine branched polyethyleneimine dendrimer (e.g., generation 0, 1, 2, 3, 4, 5, or more).

In particular embodiments, the lipid-like compound can be considered an ionizable lipid-like compound. In some embodiments, an ionizable compound can be a compound that are positively charged at acidic pH (e.g., a pH less than 7, 6, 5, or less; or a pH from about 4.5 to 7, from about 4.5 to 6.8, from about 4.5 to 6.5, from about 5.5 to 7, or from about 5.5 to 6.8) and are neutral at physiological pH (e.g., at a pH of around 7, 7.1, 7.2, 7.3, 7.4, or 7.5; or a pH from about 7.35 to 7.45 or from about 7 to 7.4).

As discussed herein, Gₘ-Cₙ compound includes a dendrimer having an m^{th} generation (Gₘ) central structure with terminal capping to provide aliphatic groups having an n number of carbon atoms (Cₙ). FIG. 3a shows a non-limiting example of a Gₘ-Cₙ compound, in within m is 0. The R group provides the aliphatic moiety attached to the terminal amino of the dendrimeric structure. Here, R is provided as —CH₂CH(OH)Cₙ₋₂H₂ₙ₋₃, in which n provides the number of carbon atoms in the R group. Other R groups can be employed, such as any useful C₁₋₂₀ optionally substituted aliphatic.

Depending on the reactions employed to install the aliphatic group, reactive products can be present. For example, if an aliphatic compound includes an oxiranyl group (e.g., an aliphatic compound that is R^{A}—CHOCH₂), then the installed R group can be —CH₂CH(OH)R^{A} (e.g., in which R^{A} can be any useful optionally substituted aliphatic group). As can be seen, the installed R group includes a —CH₂CH(OH) moiety that is a reactive product from the oxiranyl group. In another example, if an aliphatic compound includes a leaving group (e.g., an aliphatic compound that is R^{A}-X, in which X is a leaving group), then the installed R group can be —R^{A} (e.g., in which R^{A} can be any useful optionally substituted aliphatic group). FIG. 3b shows a reaction between a non-limiting dendrimeric structure (Go PAMAM) and a non-limiting aliphatic compound (epoxide having a —CHₘ₋₂H₂ₘ₋₃ group).

Any useful dendritic central structures and aliphatic groups can be employed. The central structure can include any useful repeating, branched structure. In one non-limiting embodiment, the central structure includes branched amine groups and terminal amine groups. A linker can be present between the branched and terminal amine groups. Such linkers can be any useful optionally substituted alkylene or heteroalkylene (e.g., as defined herein). FIG. 3c shows a non-limiting lipid-like compound, in which the linker is —C₂H₄C(O)NHC₂H₄— (a non-limiting optionally substituted alkylene group). Other non-limiting linkers include —Ak-, -AkC(O)Ak-, -AkC(O)NR^{N1}Ak-, -AkR_{N1}C(O)Ak-, or -AkNR^{N1}Ak-, in which Ak is a covalent bond, optionally substituted alkylene, or optionally substituted heteroalkylene, and R^{N1} is H or optionally substituted aliphatic.

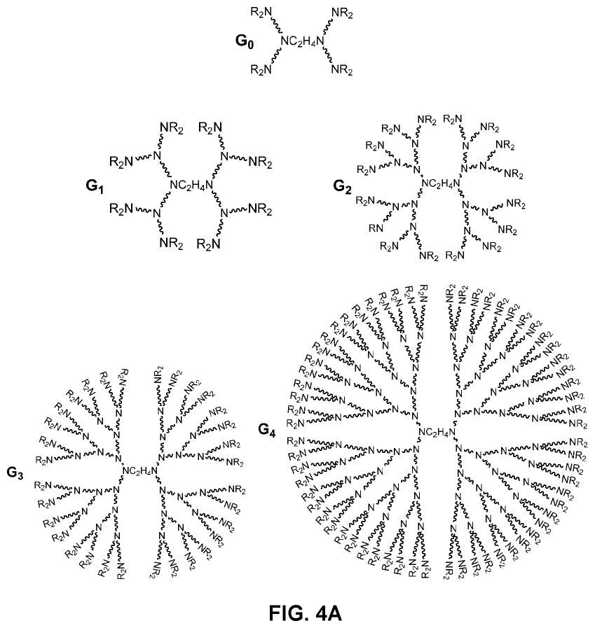

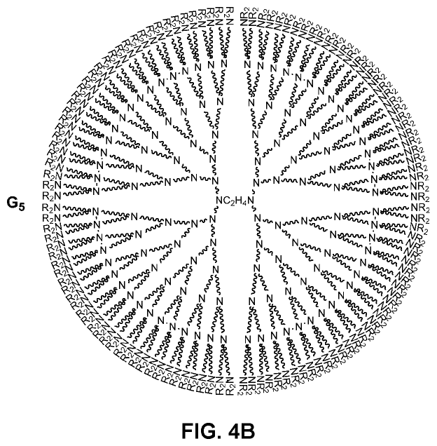

The lipid-like compound can include any useful aliphatic group (e.g., R in FIG. 3c or FIG. 4A-4B). Non-limiting aliphatic groups include any useful number of carbons (e.g., any described herein, such as from C₁₋₂₀ or ranges therebetween). In some embodiments, n in Cₙ is at least 10, 11, 12, 13, 14, 15, 16, 17, 18, 19, 20, or more. In one embodiment, the aliphatic group (R) is —CH₂CH(OH)R^{A}, in which R^{A} is optionally substituted aliphatic group. In other embodiments, R^{A} is optionally substituted alkyl, alkenyl, or alkynyl. In yet other embodiments, R is optionally substituted C₁₋₂₀, C₁₀₋₂₀, C₁₁₋₂₀, C₁₂₋₂₀, C₁₃₋₂₀, C₁₄₋₂₀, or C₁₅₋₂₀ aliphatic (e.g., optionally substituted alkyl, alkenyl, or alkynyl, as described herein).

FIG. 4A-4B provides dendrimers of varying generations. As can be seen, branched amino groups and terminal amino groups can be extended in cycles to provide repeating, branched structures (e.g., Gₘ structures, in which m is 0, 1, 2, 3, 4, 5, or more). Any of the linkers (wavy lines) in FIG. 4A-4B can include any useful optionally substituted alkylene or heteroalkylene described herein. Any of the R groups in FIG. 4A-4B can include any useful optionally substituted aliphatic described herein, in which such groups can have any useful n number of carbon atoms (Cₙ). When taking together the Gₘ and Cₙ groups, the lipid-like compound can be considered a Gₘ-Cₙ compound having any useful m or n values. Non-limiting m and n values can include, e.g., any integer in which m>0 and n<20; m=0 and n<14; or m>0 and n<20.

In particular embodiments, m can be 0, 1, 2, 3, 4, 5, 6, 7, or more. In other embodiments, n can be 1, 2, 3, 4, 5, 6, 7, 8, 9, 10, 11, 12, 13, 14, 15, 16, 17, 18, 19, 20, or more. Particular combinations can include G₀-Cₙ, G₁-Cₙ, G₂-Cₙ, G₃-Cₙ, G₄-Cₙ, or G₅-Cₙ, in which n is an integer from 1 to 20. In other embodiments, the combination can include Gₘ-C₁, Gₘ-C₂, Gₘ-C₃, Gₘ-C₄, Gₘ-C₅, Gₘ-C₆, Gₘ-C₇, Gₘ-C₈, Gₘ-C₉, Gₘ-C₁₀, Gₘ-C₁₁, Gₘ-C₁₂, Gₘ-C₁₃, Gₘ-C₁₄, Gₘ-C₁₅, Gₘ-C₁₆, Gₘ-C₁₇, Gₘ-C₁₅, Gₘ-C₁₉, or Gₘ-C₂₀, in which m is 0, 1, 2, 3, 4, 5, 6, or more. In yet other embodiments, the combination includes G₀-C₈, G₀-C₁₀, G₀-C₁₂, G₁-C₈, G₁-C₁₀, G₁-C₁₂, G₁-C₁₄, G₂-C₈, G₂-C₁₀, G₂-C₁₂, G₂-C₁₄, G₃-C₈, G₃-C₁₀, G₃-C₁₂, G₃-C₁₄, G₄-C₈, G₄-C₁₀, G₄-C₁₂, or G₄-C₁₄. In particular embodiments, lipid-like compound is not G₀-C₁₄.

In some embodiments, m≥0 and n<20. In other embodiments, m=0 and n<14. In yet other embodiments, m>0 and n<20. Other combinations include, e.g., m is 0, in which n is an integer from about 6 to about 12; m is 1, in which n is an integer from about 6 to about 12; m is 2, 3, or 4, in which n is an integer from about 6 to about 14; m is 0, 1, 2, or 3, in which n is about 8; and m is 3 or 4, in which n is about 14. In some embodiments, m is an integer from 0 to 5, and n is an integer from 6 to 18.

Any useful amount of lipid-like compound can be employed within the composition. In one embodiment, a weight ratio of the lipid-like compound to the mRNA is from about 1:1 to about 40:1 (wt:wt).

### Lipids

The composition can include one or more lipids within the outer layer. In certain instances, the lipid may be considered an amphiphile, and the outer layer can include one or more amphiphiles (e.g., any described here). Non-limiting lipids can include non-pegylated lipids, pegylated lipids, anionic lipids, cationic lipids, neutral lipids, phospholipids, ceramides, steroids, as well as others described herein. In particular embodiments, the composition includes a pegylated lipid, (e.g., DSPE-PEG), a phosphocholine (e.g., DSPC), lecithin, cholesterol, or a combination thereof

The composition can be a nanoparticle having a lipid layer. In some embodiments, nanoparticles may optionally comprise one or more lipids. In some embodiments, a nanoparticle may comprise a liposome. In some embodiments, a nanoparticle may comprise a lipid bilayer. In some embodiments, a nanoparticle may comprise a lipid monolayer. In some embodiments, a nanoparticle may comprise a micelle. In some embodiments, a nanoparticle may comprise a core comprising a polymeric matrix surrounded by a lipid layer (e.g., lipid bilayer, lipid monolayer, etc.). In some embodiments, a nanoparticle may comprise a non-polymeric core (e.g., metal particle, quantum dot, ceramic particle, bone particle, viral particle, etc.) surrounded by a lipid layer (e.g., lipid bilayer, lipid monolayer, etc.).

The percent of lipid in nanoparticles can range from 0% to 99% by weight, from 10% to 99% by weight, from 25% to 99% by weight, from 50% to 99% by weight, or from 75% to 99% by weight. In some embodiments, the percent of lipid in nanoparticles can range from 0% to 75% by weight, from 0% to 50% by weight, from 0% to 25% by weight, or from 0% to 10% by weight. In some embodiments, the percent of lipid in nanoparticles can be approximately 1% by weight, approximately 2% by weight, approximately 3% by weight, approximately 4% by weight, approximately 5% by weight, approximately 10% by weight, approximately 15% by weight, approximately 20% by weight, approximately 25% by weight, or approximately 30% by weight.

In some embodiments, lipids are oils. In general, any oil known in the art can be included in nanoparticles. In some embodiments, oil may comprise one or more fatty acid groups or salts thereof. In some embodiments, a fatty acid group may comprise digestible, long chain (e.g., C₈₋₅₀), substituted or unsubstituted hydrocarbons. In some embodiments, a fatty acid group may be a C₁₀₋₂₀ fatty acid or salt thereof. In some embodiments, a fatty acid group may be a C₁₅₋₂₀ fatty acid or salt thereof. In some embodiments, a fatty acid group may be a C₁₅₋₂₅ fatty acid or salt thereof. In some embodiments, a fatty acid group may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation.

In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linolenic, gamma-linoleic, arachidonic, gadoleic, arachidonic, eicosapentaenoic, docosahexaenoic, or erucic acid. In some embodiments, the oil is a liquid triglyceride.

Suitable oils for use include plant oils and butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

In some embodiments, a lipid is a hormone (e.g. estrogen, testosterone), steroid (e.g., cholesterol, sitosterol, bile acid), vitamin (e.g. vitamin E), phospholipid (e.g., phosphatidyl choline), sphingolipid (e.g. ceramides), or lipoprotein (e.g. apolipoprotein).

In certain embodiments, a lipid to be used in liposomes can be, but is not limited to, one or a plurality of the following: phosphatidylcholine, lipid A, cholesterol, sitosterol, dolichol, sphingosine, sphingomyelin, ceramide, glycosylceramide, cerebroside, sulfatide, phytosphingosine, phosphatidyl-ethanolamine, phosphatidylglycerol, phosphatidylinositol, phosphatidylserine, cardiolipin, phosphatidic acid, and lyso-phophatides. In certain embodiments, a targeting moiety or a targeting ligand can be conjugated to the surface of a liposome.

In some embodiments, nanoparticle-stabilized liposomes are used to deliver the cargo. By allowing small charged nanoparticles (e.g., 1 nm-30 nm) to adsorb on liposome surface, liposome-nanoparticle complexes have not only the merits of bare liposomes, but also tunable membrane rigidity and controllable liposome stability. When small charged nanoparticles approach the surface of liposomes carrying either opposite charge or no net charge, an electrostatic or charge-dipole interaction between nanoparticles and membrane attracts the nanoparticles to stay on the membrane surface, being partially wrapped by lipid membrane. This induces local membrane bending and globule surface tension of liposomes, both of which can enable tuning of membrane rigidity. Moreover, adsorbed nanoparticles form a charged shell which protects liposomes against fusion, thereby enhancing liposome stability. In certain embodiments, small nanoparticles are mixed with liposomes under gentle vortex, and the nanoparticles stick to liposome surface spontaneously. In specific embodiments, small nanoparticles can be, but are not limited to, polymeric nanoparticles, metallic nanoparticles, inorganic or organic nanoparticles, hybrids thereof, and/or combinations thereof.

In some embodiments, liposome-polymer nanoparticles are used to deliver a combination of one or more inhibitory nucleic acids and one or more nucleic acids encoding a protein or polypeptide.

In some embodiments, a core can be surrounded by an outer layer or coating layer (e.g., liposome, lipid monolayer, micelle, etc.). In one embodiment, the lipid monolayer shell comprises an amphiphilic compound. In another embodiment, the amphiphilic compound is lecithin. In another embodiment, the lipid monolayer is stabilized.

Specific examples of amphiphilic compounds include, but are not limited to, phospholipids, such as 1,2-distearoyl-sn-glycero-3-phosphoethanolamine (DSPE), 1,2-dioleoyl-sn-glycero-3-phosphoethanolamine (DOPE), 1,2-dimyristoyl-sn-glycero-3-phosphoethanolamine (DMPE), 1,2-dipalmitoyl-sn-glycero-3-phosphoethanolamine (DPPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), dipalmitoylphosphatidylcholine (DPPC), distearoylphosphatidylcholine (DSPC), dioleoylphosphatidylcholine (DOPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), palmitoyloleoyl-phosphatidylethanolamine (POPE), and palmitoyloleyol-phosphatidylglycerol (POPG), which can be optionally incorporated at a ratio of between 0.01-60 (weight lipid/w polymer) or between 0.1-30 (weight lipid/w polymer). Phospholipids that may be used include, but are not limited to, phosphatidic acids, phosphatidylcholines with both saturated and unsaturated lipids, phosphatidylethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines, such as dioleoylphosphatidylcholine, dimyristoylphosphatidylcholine, dipentadecanoylphosphatidylcholine, dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphatidylcholine (DLPC); and phosphatidylethanolamines, such as dioleoylphosphatidylethanolamine or 1-hexadecyl-2-palmitoylglycerophosphoethanolamine. Synthetic phospholipids with asymmetric acyl chains (e.g., with one acyl chain of 6 carbons and another acyl chain of 12 carbons) may also be used.

In a particular embodiment, an amphiphilic component that can be used to form an amphiphilic layer is lecithin, and, in particular, phosphatidylcholine. Lecithin is an amphiphilic lipid and, as such, forms a phospholipid bilayer having the hydrophilic (polar) heads facing their surroundings, which are oftentimes aqueous, and the hydrophobic tails facing each other. Lecithin has an advantage of being a natural lipid that is available from, e.g., soybean, and already has FDA approval for use in other delivery devices.

In certain embodiments, the amphiphilic layer of the nanoparticle, e.g., the layer of lecithin, is a monolayer, meaning the layer is not a phospholipid bilayer, but exists as a single continuous or discontinuous layer around, or within, the nanoparticle. A monolayer has the advantage of allowing the nanoparticles to be smaller in size, which makes them easier to prepare. The amphiphilic layer can be “associated with” the nanoparticle, meaning it is positioned in some proximity to the core.

In covering the core, the amphiphilic compounds form a tightly assembled monolayer, bilayer, or multilayer around the core. This layer can prevent the carried agents (or cargos) from freely diffusing out of the nanoparticle, thereby enhancing the encapsulation yield and slowing drug release. Moreover, the amphiphilic layer can reduce water penetration rate into the nanoparticle, which slows hydrolysis rate of the biodegradable polymers, thereby increasing particle stability and lifetime.

In further embodiments, targeting ligands can be conjugated to the lipid-like compound, lipid, or other amphiphilic component prior to incorporating them into the nanoparticle. Alternatively, targeting ligands can be conjugated to the polymeric component of the nanoparticles. Non-limiting targeting ligands are described herein.

### Targeting Ligands and Targeting Moieties

The compositions herein can include binding moieties or targeting moieties that specifically bind to a target cell or tissue. Representative targeting moieties include, but are not limited to, antibodies and antigen binding fragments thereof, aptamers, peptides, small molecules, as well as others described herein. Typically, the binding moiety is displayed on the outer layer of the particle. The outer layer can serve as a shield to prevent the particles from being recognized by a subject's immune system thereby increasing the half-life of the nanoparticles in the subject. The particles also contain a hydrophobic core. In some embodiments, the hydrophobic core is made of a biodegradable material. The inner core carries therapeutic payloads and releases the therapeutic payloads at a sustained rate after systemic, intraperitoneal, oral, pulmonary, or topical administration. The nanoparticles also optionally include a detectable label, for example a fluorophore or NMR contrast agent that allows visualization of nanoparticles within plaques.

In some embodiments, the composition can include any useful targeting ligand for use with a cell (e.g., a tumor cell). The targeting ligand can be used to target a receptor (e.g., a chemokine receptor), a saccharide, or other extracellular component present on a target cell. In one embodiment, the targeting ligand includes: (i) a targeting moiety configured to bind to an outer portion of a cell, (ii) a lipid configured to interact with or form a portion of the outer layer of a delivery vehicle; and (iii) and a linker disposed between the targeting moiety and the lipid.

In some embodiments, the targeting ligand is configured to target a chemokine receptor. In particular embodiments, the chemokine receptor is CXCR4, and the targeting ligand includes a CXCR4-targeting ligand. In turn, the CXCR4-targeting ligand includes a CXCR4-targeting moiety bound to a lipid, as well as an optional linker (e.g., a PEG linker) disposed between the CXCR4-targeting moiety and the lipid. The CXCR4-targeting moiety can includes a sequence having at least 80% sequence identity to KGVSLSYRCRYSLSVGK (SEQ ID NO: 1) or a fragment thereof. Exemplary CXCR4 protein sequences are provided at NCBI Accession Nos. NP_001008540.1, NP 001334985.1, NP_001334988.1, NP_001334989.1, and NP_003458.1.

In other embodiments, the chemokine receptor is glypican-3 (GPC3), and the targeting ligand includes a GPC3-targeting ligand. In turn, the GPC3-targeting ligand includes a GPC3-targeting moiety bound to a lipid, as well as an optional linker (e.g., a PEG linker) disposed between the GPC3-targeting moiety and the lipid. The GPC3-targeting moiety can includes a sequence having at least 80% sequence identity to THVSPNQGGLPS (SEQ ID NO: 7), RLNVGGTYFLTTRQ (SEQ ID NO: 8), SNDRPPNILQKR (SEQ ID NO: 9), or a fragment thereof. Other non-limiting GPC3-targeting moieties include any in Grega et al., Am. J. Nucl. Med. Mol. Imaging 12(4), 113-121 (2022). Exemplary GPC3 protein sequences are provided at NCBI Accession Nos. NP_001158089.1, NP_001158091.1, and NP_004475.1

Other non-limiting targeting moieties can include a protein (e.g., an antibody, a glycoprotein, and the like, such as transferrin), a peptide (e.g., any described herein), an aptamer (e.g., a CD133 aptamer and the like), a nucleic acid (e.g., a nucleic acid-based ligand), a monosaccharide (e.g., galactose, glucose, mannose, and the like), a polysaccharide (e.g., hyaluronic acid, mannan, and the like), a carbohydrate (e.g., N-acetylgalactosamine (GalNAc), sialic acid, and the like), a vitamin (e.g., folate, biotin, etc.), a small molecule, and the like. Any of these groups can be employed as a targeting moiety. In particular embodiments, the targeting ligand can include a targeting moiety (e.g., any described herein) that can be optionally attached to a lipid (e.g., any described herein) or an aliphatic group (e.g., any described herein), by way of a linker. The type and density of targeting ligands can be selected to provide engineered NPs for improving specific delivery of the mRNA to the targeted sites, and thus improve the therapeutic efficacy and avoid collateral cytotoxicity.

The targeting moiety can be an antibody or antigen binding fragment thereof. The targeting moieties should have an affinity for a cell-surface receptor or cell-surface antigen on the target cells. The targeting moieties may result in internalization of the particle within the target cell.

In certain embodiments, the targeting moiety can include a peptide. Non-limiting peptides include, e.g., RED, oct-arginine (RRRRRRRR, SEQ ID NO: 10), TAT (GRKKRRQRRRPQ, SEQ ID NO: 11), penetratin (RQIKIWFQNRRMKWKK, SEQ ID NO: 12), CRQTKN peptide (SEQ ID NO: 13), CRGDK peptide (SEQ ID NO: 14), CCGKRK peptide (SEQ ID NO: 15), GEl1 (YHWYGYTPQNVI, SEQ ID NO: 16), TfR-T12 (THRPPMWSPVWP, SEQ ID NO: 17), as well as other cell-penetrating peptides.

In other embodiments, the CXCR4-targeting moiety can include a peptide or a non-peptide (e.g., a small molecule, such as AMD3100 (plerixafor), AMD11070/AMD070 (Genzyme), ITlt (isotiourea-lt), MSX-122 (Metastatix), TG-0054 (TaiGen), and the like). Non-limiting peptide- or protein-based targeting moieties include Ac-TZ14011, BL-8040/BKT140 (BioLineRx, Biokine), POL6326 (Polyphor), LY2510924 (Eli Lilly), hz515H₇ (humanized monoclonal IgG1 anti-CXCR4 antibody), LY2624587 (humanized anti-CXCR4 monoclonal antibody; Eli Lilly), PF-06747143 (humanized IgG1 monoclonal antibody (mAb; Pfizer), uloculpumab (human IgG4 monoclonal anti-CXCR4 antibody, BMS-936564/MDX1338; Bristol Myers Squibb), and the like. Yet other targeting moieties include, e.g., one or more of the following:

(SEQ ID NO: 20)
KGVSLSYRKRYSLSVGK;

(KGVSLSYR)2K—NH2 (SEQ ID NO: 21, including the following modifications: amide bridge=Lys9-Arg8, Lys-9=C-terminal amide);

(SDF-1 (1-17), SEQ ID NO: 22)
KPVSLSYRCPCRFFESH;
(SDF-1 (1-68), SEQ ID NO: 23)
KPVSLSYRCPCRFFESHVARANVKHL
KILNTPNCALQIVARLKNNNRQVCID
PKLKWIQEYLEKALNK;
(L5H, SEQ ID NO: 24)
KPVSHSYRCPCRF;
(SDF-1(1-9) P2G dimer, SEQ ID NO: 25)
(KGVSLSYRC)₂;
(ASLW, SEQ ID NO: 26)
ASLWLSYRCPCRFFESH;

H-RRWC¹YRKC²YKGYC²YRKC¹R—NH₂ (T22 having a C¹-C¹ disulfide bridge and a C²-C² disulfide bridge, SEQ ID NO: 27);
H—RRX¹CYRKK*PYRX²CR—OH (T140 having a C-C disulfide bridge, in which X¹ is L-3-(2-naphthyl)alanine (Nal), X² is L-citrulline (Cit), and K* is D-K, SEQ ID NO: 28);
H-RRX¹CYX²K*X²PYRX²CR—NH² (T14012 having a C-C disulfide bridge, in which X¹ is L-3-(2-naphthyl)alanine (Nal), X² is L-citrulline (Cit), and K* is D-K, SEQ ID NO: 29);
H-RRWCYRKK*PYRX²CR—OH (T134 having a C-C disulfide bridge, in which X² is L-citrulline (Cit) and K* is D-K, SEQ ID NO: 30);
H-RRX¹CYX²KK*PYRX²CR—NH₂ (TN14003 having a C-C disulfide bridge, in which X² is L-citrulline (Cit) and K* is D-K, SEQ ID NO: 31);

(FC131, SEQ ID NO: 32)
cyclo(-D-Tyr-Arg-Arg-Nal-Gly-);
(FC122, SEQ ID NO: 33)
cyclo(-D-Tyr-D-MeArg-Arg-Nal-Gly-);
(SEQ ID NO: 34)
cyclo(-Nal-Gly-D-Tyr-Arg-Arg-);
(ALX40-4C, in which R* is D-R, SEQ ID NO: 35)
Ac-(R*)₉;
(V1 [vMIP-11 (1-21)], SEQ ID NO: 36)
LGASWHRPDKCCLGYQKRPLP;
(DV1 (all D-amino acids), SEQ ID NO: 37)
D-[LGASWHRPDKCCLGYQKRPLP];
(vMIP-11 (1-71), SEQ ID NO: 38)
LGASWHRPDKCCLGYQKRPLPQVLLSSWYPTSQLCSKP
GVIFLTKRGRQVCADKSKDWVKKLMQQLPVTAR;
or
(D-amino acids in bold, SEQ ID NO: 39)
LGASWHRPDKCCLGYQKRPLPQVLLSSWYPTSQLCS
KPGVIFLTKRGRQVCADKSKDWVKKLMQQLPVTAR.

The targeting moiety can specifically recognize and bind to a target molecule specific for a cell type, a tissue type, or an organ. The target molecule can be a cell surface polypeptide, lipid, or glycolipid. The target molecule can be a receptor that is selectively expressed on a specific cell surface, a tissue, or an organ. Cell-specific markers can be for specific types of cells including, but not limited to, stem cells, skin cells, blood cells, immune cells, muscle cells, nerve cells, cancer cells, virally infected cells, and organ specific cells. The cell markers can be specific for endothelial, ectodermal, or mesenchymal cells. Representative cell specific markers include, but are not limited to cancer specific markers.

Exemplary targets (or target molecules) include prostate-specific membrane antigen (PSMA); GAH; myosin 14 (MYH14); human epidermal growth factor receptor 2 (HER2); transferrin (Tf) receptor; epithelial cell adhesion molecule (EpCAM); globular Clq receptor (gClqR) or p32; nucleolin; avP3/5 integrin; collagen IV; fibronectin; folic acid (FA) receptor; and mitochondria. Exemplary methods and moieties for targeting cancer cells, including proteins, peptides, nucleic acid-based ligands and small molecules, are described herein and in Bertrand et al., Adv. Drug Deliv. Rev. 66, 2-25 (2014), see, e.g., Table 2 and Section 3.4 entitled “Targeting Ligands” therein).

In one embodiment, the targeting moiety is a peptide. Specifically, a targeted peptide can be, but is not limited to, one or more of the following: RGD, iRGD (CRGDX₁GPX₂C, SEQ ID NO: 18, where X₁ is K or R and X₂ is D or E), LyP-1, P3 (CKGGRAKDC, SEQ ID NO: 19), or their combinations at various molar ratios. The targeting peptide can be covalently associated with a polymer, a lipid, an amphiphile, and the like, in which covalent association can be mediated by a linker. The peptides can allow for targeting of actively growing (angiogenic) vascular endothelial cells. Those angiogenic endothelial cells frequently appear in metabolic tissues such as adipose tissues.

The targeting moiety can be an antibody or an antigen-binding fragment thereof. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. The antigen binding portion of the antibody can be any portion that has at least one antigen binding site, such as Fab, F(ab′)₂, dsFv, sFv, nanobodies, diabodies, and triabodies. In certain embodiments, the antibody is a single chain antibody.

Aptamers are oligonucleotide or peptide sequences with the capacity to recognize virtually any class of target molecules with high affinity and specificity. Aptamers can bind to targets such as small organics, peptides, proteins, cells, and tissues. Unlike antibodies, some aptamers can exhibit stereoselectivity. The aptamers can be designed to bind to specific targets expressed on cells, tissues or organs.

The nanoparticles can contain one or more polymer conjugates containing end-to-end linkages between the polymer and a moiety, or one or more amphiphile conjugates containing end-to-end linkages between the amphiphile and a moiety. The moiety can be a targeting moiety, a detectable label, or a therapeutic, prophylactic, or diagnostic agent. For example, a polymer conjugate can be a PLGA-PEG-TM, in which™ is the targeting moiety. In another example, an amphiphile conjugate can be a DSPE-PEG-TM, in which™ is the target moiety. The additional targeting elements may refer to elements that bind to or otherwise localize the nanoparticles to a specific locale. The locale may be a tissue, a particular cell type, or a subcellular compartment. The targeting element of the nanoparticle can be an antibody or antigen binding fragment thereof, an aptamer, or a small molecule (less than 500 Daltons). The additional targeting elements may have an affinity for a cell-surface receptor or cell-surface antigen on a target cell and result in internalization of the particle within the target cell.

Targeting ligands can be present in any useful amount. In particular embodiments, the density of targeting ligands for the delivery vehicle is between about 3% to 10%.

### Linkers

Linkers can be present between two components (e.g., lipid and targeting moiety, lipid and PEG moiety, dendrimer and aliphatic moiety, two polymer components, and the like). Linkers can include a bond (e.g., a covalent bond); an amino acid; a plurality of amino acids; a nucleotide; a plurality of nucleotides; an optionally substituted alkylene; an optionally substituted heteroalkylene (e.g., poly(ethylene glycol), such as —(OCH₂CH₂)ₙ—, in which n is an integer of 1 to 100); an optionally substituted arylene; or an optionally substituted heteroarylene.

The linker can include one or more chemical signatures. In one embodiment, the chemical signature includes a click-chemistry signature, which arises from reacting a click-chemistry reaction pair (e.g., any described herein). Non-limiting examples of click-chemistry signatures include a triazole, an unsaturated six-member ring, a covalent bond, and the like.

In another embodiment, the chemical signature can include a reaction signature, which arises from reacting a cross-linker reaction pair. Non-limiting examples of cross-linker reaction pairs include those for forming a covalent bond between a carboxyl group (e.g., —CO₂H) and an amino group (e.g., —NH₂); or between an imido group (e.g., maleimido or succinimido) and a thiol group (e.g., —SH); or between an epoxide group and a thiol group (e.g., —SH); or between an epoxide group and an amino group (e.g., —NH₂); or between an ester group (e.g., —CO₂R, in which R is an organic moiety, such as optionally substituted alkyl, aryl, etc.) and an amino group (e.g., —NH₂); or between an carbamido group (e.g., —NHC(O)Het, where Het is a N-containing heterocyclyl) and an amino group (e.g., —NH₂); or between a phospho group (e.g., —P(O)(OH)₂) and an amino group (e.g., —NH₂), such as 1-ethyl-3-(3-dimethylaminopropyl) carbodiimide (EDC) and dicyclohexylcarbodiimide (DCC), optionally used with N-hydroxysuccinimide (NHS) and/or N-hydroxysulfosuccinimide (sulfo-NHS). Other cross-linkers include those for forming a covalent bond between an amino group (e.g., —NH₂) and a thymine moiety, such as succinimidyl-[4-(psoralen-8-yloxy)]-butyrate (SPB); a hydroxyl group (e.g., —OH) and a sulfur-containing group (e.g., free thiol, —SH, sulfhydryl, cysteine moiety, or mercapto group), such as p-maleimidophenyl isocyanate (PMPI); between an amino group (e.g., —NH₂) and a sulfur-containing group (e.g., free thiol, —SH, sulfhydryl, cysteine moiety, or mercapto group), such as succinimidyl 4-(p-maleimidophenyl)butyrate (SMPB) and/or succinimidyl 4-(N-maleimidomethyl)cyclohexane-1-carboxylate (SMCC); between a sulfur-containing group (e.g., free thiol, —SH, sulfhydryl, cysteine moiety, or mercapto group) and a carbonyl group (e.g., an aldehyde group, such as for an oxidized glycoprotein carbohydrate), such as N-beta-maleimidopropionic acid hydrazide-trifluoroacetic acid salt (BMPH), 3-(2-pyridyldithio)propionyl hydrazide (PDPH), and/or a 3-(2-pyridyldithio)propionyl group (PDP); and between a maleimide-containing group and a sulfur-containing group (e.g., free thiol, —SH, sulfhydryl, cysteine moiety, or mercapto group). Yet other cross-linkers include those for forming a covalent bond between two or more unsaturated hydrocarbon bonds, e.g., mediated by radical polymerization, such as a reaction of forming a covalent bond between a first alkene group and a second alkene group (e.g., a reaction between acrylate-derived monomers to form a polyacrylate, polyacrylamide, etc.).

The linker can include one or more reaction pairs. In one embodiment, the reaction pair is one of a click-chemistry reaction pair, which can include a first click-chemistry group and a second click-chemistry group that reacts with that first click-chemistry group. Exemplary click-chemistry groups include, e.g., a click-chemistry group, e.g., one of a click-chemistry reaction pair selected from the group consisting of a Huisgen 1,3-dipolar cycloaddition reaction between an alkynyl group and an azido group to form a triazole-containing linker; a Diels-Alder reaction between a diene having a 471 electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 27L electron system (e.g., an optionally substituted alkenyl group or an optionally substituted alkynyl group); a ring opening reaction with a nucleophile and a strained heterocyclyl electrophile; and a splint ligation reaction with a phosphorothioate group and an iodo group; and a reductive amination reaction with an aldehyde group and an amino group.

The linker can include one or more reactive groups. Exemplary reactive groups include an amino (e.g., —NH₂), a thio (e.g., a thioalkoxy group or a thiol group), a hydroxyl, an ester (e.g., an acrylate), a carboxyl (e.g., —CO₂H or a deprotonated form thereof), an imido (e.g., a maleimido or a succinimido), an epoxide, an isocyanate, an isothiocyanate, an anhydride, an amido, a carbamido (e.g., a urea derivative), an azide, an optionally substituted alkynyl, or an optionally substituted alkenyl.

In other embodiments, the linker can include a binding reaction signature, which arises from reacting a binding reaction pair. Exemplary binding groups and binding reaction pairs include those for forming a covalent bond between biotin and avidin, biotin and streptavidin, biotin and neutravidin, desthiobiotin and avidin (or a derivative thereof, such as streptavidin or neutravidin), hapten and an antibody, an antigen and an antibody, a primary antibody and a secondary antibody, and lectin and a glycoprotein.

### Formulations

The compositions herein can be provided in a formulation. In one embodiment, the formulation can include a therapeutically effective amount of a composition (e.g., any described herein) and a pharmaceutically acceptable excipient. The formulation can be formulated for injection, implantation, and the like. The methods described herein include the use of formulations including an mRNA encoding a tumor suppressor complexed with a delivery vehicle as an active ingredient, as well as including one or more excipients including an inactive ingredient.

Formulations typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., an immunotherapy agent as described herein.

Formulations are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerol, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Formulations suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, the formulation can include isotonic agents, for example, sugars, polyalcohols such as mannitol, sorbitol, and sodium chloride. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, possible methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents and/or adjuvant materials can be included as part of the composition. The tablets, pills, capsules, troches, and the like can contain any of the following ingredients, or compounds of a similar nature: a binder, such as microcrystalline cellulose, gum tragacanth, or gelatin; an excipient, such as starch or lactose; a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavoring agent, such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

The pharmaceutical compositions can be included in a container, pack, or dispenser together with instructions for administration.

### Methods and Uses Thereof

The present disclosure encompasses methods of using and making any composition herein. In one embodiment, the method can include treating a cancer by administering a therapeutically effective amount of a composition (e.g., any described herein) or a formulation (e.g., any described herein) to a subject in need thereof. The cancer can be any described herein, such as a p53-deficient cancer or a cancer associated with loss of p53 expression or activity. In other embodiments, the cancer is liver cancer (e.g., hepatocellular carcinoma (HCC) or cholangiocarcinoma), lung cancer (e.g., non-small cell lung cancer (NSCLC)), colon cancer, pancreatic cancer, prostate cancer, breast cancer, glioblastoma, as well as primary or metastatic forms of any of these (e.g., liver metastases from a p53-deficient cancer, such as colon, lung, pancreatic cancer., etc.)), or any described herein.

In yet other embodiments, the cancer is associated with loss of expression or activity of a tumor suppressor. In some embodiments, the tumor suppressor-encoding mRNAs comprise mRNAs encoding p53 protein, and the subject has a cancer associated with loss of expression or activity of p53. Determining that a subject has a cancer that is associated with loss of a tumor suppressor can be done using any method known in the art, e.g., obtaining a sample comprising tumor cells, and detecting the presence of a mutation or loss of a tumor suppressor in the cells, e.g., by sequencing DNA of the tumor cells and detecting a mutation that is known to be associated with oncogenesis, or by detecting a decreased level or activity of the tumor suppressor protein as compared to a reference, e.g., a reference that represents a level or activity of the protein in a normal, non-cancerous cell of the same type as the tumor cell (i.e., a cell from the same kind of tissue, a non-cancerous part of the same tissues in the same individual or in an individual who doesn't have cancer).

In some embodiments, the delivery vehicle (e.g., nanoparticle) is complexed with mRNAs that encode a single tumor suppressor; in other embodiments, the vehicle is complexed with mRNAs coding for multiple tumor suppressors. In some embodiments, the methods include administering a plurality vehicle-RNA complexes that include vehicles complexed with two or more mRNAs, e.g., wherein the vehicles are each complexed with only a single kind of mRNA (i.e., each vehicle is complexed with mRNA encoding one tumor suppressor), or wherein the vehicles are each complexed with two or more kinds of mRNAs (i.e., the vehicles are each complexed with mRNAs encoding two or more tumor suppressors).

Any method herein can include determining a subject that would benefit from treatment. In one embodiment, the method can include determining that a subject has a cancer that is associated with loss of a tumor suppressor and then delivering an mRNA encoding a tumor suppressor beneficial to treat the cancer, e.g., to the tumor in the subject.

As used herein, cancer refers to a condition in which cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. Pathologic hyperproliferative cells can occur in disease states characterized by malignant tumor growth.

Cancer or neoplasms can include malignancies of various organ systems, such as affecting lung, breast, thyroid, lymphoid, gastrointestinal, and genito-urinary tract, as well as adenocarcinomas that include malignancies such as most colon cancers, renal-cell carcinoma, prostate cancer, and/or testicular tumors, non-small cell carcinoma of the lung, cancer of the small intestine, and cancer of the esophagus.

As used herein, the term “carcinoma” refers to malignancies of epithelial or endocrine tissues, including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An adenocarcinoma refers to a carcinoma derived from glandular tissue or in which the tumor cells form recognizable glandular structures.

As used herein, the term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. In some instances, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus L et al., Crit. Rev. Oncol. Hemotol. 11, 267-297 (1991)); lymphoid malignancies include, but are not limited to, acute lymphoblastic leukemia (ALL) including B-lineage ALL and T-lineage ALL, chronic lymphocytic leukemia (CLL), prolymphocytic leukemia (PLL), hairy cell leukemia (HLL), and Waldenstrom's macroglobulinemia (WM). Additional forms of malignant lymphomas include, but are not limited to, non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease, and Reed-Sternberg disease.

In some embodiments, the methods can include modulating an interaction between a cell lacking a tumor suppressor (e.g., a tumor) and an immune cell. The method can include: administering a therapeutically effective amount of a composition (e.g., any described herein) or a formulation (e.g., any described herein) to a subject in need thereof. Non-limiting immune cells include an NK cell, a T cell, and/or a tumor-associated macrophage (TAM).

The present disclosure also encompasses methods of making a composition. In some embodiments, the method includes: complexing an mRNA with a lipid-like compound of Gₘ-Cₙ in an acidic environment, wherein m≥0 and n<20, in the presence of an optional water-insoluble polymer, thereby forming a core; and surrounding the core with an outer layer including a lipid, a pegylated lipid, or a target ligand, thereby providing the composition. In other embodiments, the method includes: complexing an mRNA with a lipid-like compound of Gₘ-Cₙ in an acidic environment, wherein m≥0 and n<20; forming a core around the mRNA and the lipid-like compound in the presence of a water-insoluble polymer; and surrounding the core with an outer layer including a lipid, a pegylated lipid, or a target ligand, thereby providing the composition.

In particular embodiments, acidic environments can be used during complexation of the cargo to the lipid-like compound. In one embodiment, the acidic environment includes a pH from about 4 to about 2. In another embodiment, the acidic environment can include a buffered solution. In other embodiments, the ratio of cargo to other components (e.g., lipid-like compound, lipid, polymer, amphiphile, and the like) can be optimized.

Introducing components into the composition can be performed in any useful manner. In one instance, a particular order can be employed. For example and without limitation, an initial operation can include complexing a cargo with the lipid-like compound in an acidic, aqueous solvent, thereby forming a core. Subsequent operations can include surrounding the core with a lipid, such as by introducing the core to a solvent (e.g., an aqueous solvent, such as water) including one or more lipids (e.g., a pegylated lipid) and, optionally, a targeting ligand (e.g., a CXCR4-targeting ligand). In certain embodiments, surrounding the core can include stirring the solvent (e.g., at a rate of about 500 to 1500 rpm). If a polymer is employed within the core, then forming the polymeric core can include introducing a water-insoluble polymer in a solvent (e.g., an organic solvent) to the complexed cargo. In some embodiments, the method can provide any composition described herein (e.g., a lipid particle, a hybrid polymer-lipid particle, a micelle, a liposome, and the like).

Methods of making formulations are also encompassed by the present disclosure. For instance and without limitation, any composition herein can be formulated with one or more pharmaceutically acceptable excipients. The dosage or amount of composition within the formulation can be provide a therapeutically effective amount to treat any condition described herein.

### Immunotherapy and Other Combination Therapies

The present disclosure encompasses the use of a composition (e.g., a delivery vehicle, such as any described herein) for treatment of a subject. Such a treatment can be combined with one or more other treatments that, when combined, could provide therapeutic benefit to the subject. In one non-limiting embodiment, the methods herein can include administering a therapeutically effective amount of a cargo with a combination therapy to a subject in need thereof. In particular embodiments, the mRNA includes an mRNA encoding a tumor suppressor protein (e.g., p53 or any other protein described herein), and the method includes a method of treating cancer (e.g., characterized by loss or decreased function of p53, by loss of p53 expression, by loss of p53 activity, and/or by mutation of p53). In some embodiments, the cancer is a p53-deficient cancer, and the combination therapy is a therapy that would be useful for treating such a cancer.

Any useful combination therapy can be used in conjunction with administering a cargo with a delivery vehicle (e.g., any described herein). In some embodiments, the combination therapy can include one or more therapies beneficial for treating cancer. In other embodiments, the combination therapy can include immunotherapy, anti-angiogenesis therapy, radiotherapy, and the like.

In some embodiments, the methods also include co-administering an immunotherapy agent to a subject who is treated with a method or composition described herein. Immunotherapy agents include those therapies that target tumor-induced immune suppression (see, e.g., Stewart and Smyth, Cancer Metastasis Rev. 30(1), 125-140 (2011)).

In one instance, the combination therapy can include immunotherapy, and the method can further include administering a therapeutically effect amount of at least one immune checkpoint inhibitor to the subject. In some embodiments, at least one immune checkpoint inhibitor is an anti-PD-1 (aPDI) antibody, an anti-PD-L1 (aPD-L1) antibody, an anti-CTLA-4 antibody, an anti-TIM3 antibody, an anti-LAG3 antibody, an anti-CD137 antibody, or an anti-CD40 antibody. Administration of the immune checkpoint inhibitor can include any useful regimen, e.g., at a timepoint before, after, or during said administering a delivery vehicle, a composition, at least one anti-angiogenesis inhibitor, and/or radiation, or other therapies described herein.

Exemplary anti-PD-1 antibodies that can be used in the methods described herein include those that bind to human PD-1. An exemplary PD-1 protein sequence is provided at NCBI Accession No. NP_005009.2. Exemplary antibodies are described in U.S. Pat. Nos. 8,008,449 and 9,073,994 and U.S. Pat. Pub. No. 2011/0271358, including PF-06801591, AMP-224, AZD7789 (Astra Zeneca), BI 754091, FS222 (F-Star Therapeutics), JS001, LB1410 (L&L biopharma Co., Ltd.), LY3300054 (Eli Lilly and Co.), MEDIO680, PDR001, REGN2810, R07121661 (Hoffmann-La Roche), SHR-1210, Sym-021 (Symphogen AS), TSR-042 (dostarlimab; TSR and GSK), pembrolizumab, nivolumab, avelumab, pidilizumab, tislelizumab (BGB-A317; Bei Gene), and atezolizumab.

Exemplary anti-PD-L1 antibodies that can be used in the methods described herein include those that bind to human PD-Li. Exemplary PD-Ll protein sequences are provided at NCBI Accession Nos. NP_001254635.1, NP_001300958.1, and NP_054862.1. Exemplary antibodies are described in U.S. Pat. Pub. No. 2017/0058033 and Int. Pub. Nos. WO 2016/061142A1, WO 2016/007235A1, WO 2014/195852A1, and W02013/079174A1, including BMS-936559 (MDX-1105), FAZ053, KN035, atezolizumab (Tecentriq, MPDL3280A), avelumab (Bavencio), and durvalumab (Imfinzi, MEDI-4736).

Exemplary anti-CD40 antibodies that can be used in the methods described herein include those that bind to human CD40. Exemplary CD40 protein precursor sequences are provided at NCBI Accession Nos. NP_001241.1, NP_690593.1, NP_001309351.1, NP_001309350.1, and NP_001289682.1. Exemplary antibodies include those described in Int. Pub. Nos. WO 2002/088186, WO 2007/124299, WO 2011/123489, WO 2012/149356, WO 2012/111762, and WO2014/070934; U.S. Pat. Pub. Nos. 2013/0011405, 2007/0148163, 2004/0120948, and 2003/0165499; and U.S. Pat. No. 8,591,900; including dacetuzumab, lucatumumab, bleselumab, teneliximab, ADC-1013, CP-870,893, Chi Lob 7/4, HCD122, SGN-4, SEA-CD40, BMS-986004, and APX005M. In some embodiments, the anti-CD40 antibody is a CD40 agonist and not a CD40 antagonist.

In some embodiments, these immunotherapies may primarily target immunoregulatory cell types, such as regulatory T cells (Tregs) or M2 polarized macrophages, e.g., by reducing number, altering function, or preventing tumor localization of the immunoregulatory cell types. For example, Treg-targeted therapy includes anti-GITR monoclonal antibody (TRX518), cyclophosphamide (e.g., metronomic doses), arsenic trioxide, paclitaxel, sunitinib, oxaliplatin, PLX4720, anthracycline-based chemotherapy, daclizumab (anti-CD25), immunotoxin (e.g., Ontak or denileukin diftitox), lymphoablation (e.g., chemical or radiation lymphoablation), agents that selectively target the VEGF-VEGFR signalling axis (e.g., such as VEGF blocking antibodies (e.g., bevacizumab), or inhibitors of VEGFR tyrosine kinase activity (e.g., lenvatinib)), agents that target ATP hydrolysis (e.g., using ectonucleotidase inhibitors, e.g., ARL67156 (6-N,N-diethyl-D-0,7-dibromomethyleneATP trisodium salt), 8-(4-chlorophenylthio) cAMP (pCPT-cAMP) and a related cyclic nucleotide analogue (8-[4-chlorophenylthio]cGMP; pCPT-cGMP), and those described in Int. Pub. No. WO 2007/135195, as well as antibodies (e.g., monoclonal antibodies) against CD73 or CD39). Docetaxel also has effects on M2 macrophages. See, e.g., Zitvogel et al., Immunity 39, 74-88 (2013).

In another example, M2 macrophage-targeted therapy includes clodronate-liposomes (Zeisberger et al., Br. J. Cancer 95, 272-281 (2006)), DNA-based vaccines (Luo et al., J. Clin. Invest. 116(8), 2132-2141 (2006)), and M2 macrophage-targeted, pro-apoptotic peptides (Cieslewicz et al., Proc. Natl. Acad. Sci. USA 110(40), 15919-15924 (2013)). Other immunotherapies target the metabolic processes of immunity, and include adenosine receptor antagonists and small molecule inhibitors, e.g., istradefylline (KW-6002) and SCH-58261; indoleamine 2,3-dioxygenase (IDO) inhibitors, e.g., small molecule inhibitors (e.g., 1-methyl-tryptophan (1MT), 1-methyl-d-tryptophan (D1MT), and Toho-1), IDO-specific siRNAs, natural products (e.g., brassinin or exiguamine) (see, e.g., Munn, Front. Biosci. (Elite Ed.) 4, 734-745 (2012)), or monoclonal antibodies that neutralize the metabolites of IDO, e.g., mAbs against N-formyl-kynurenine.

In some embodiments, the immunotherapies may antagonize the action of cytokines and chemokines, such as IL-10, TGF-beta, IL-6, CCL2, and others that are associated with immunosuppression in cancer. For example, TGF-beta neutralizing therapies include anti-TGF-beta antibodies (e.g., fresolimumab, infliximab, lerdelimumab, GC-1008m, and the like), antisense oligodeoxynucleotides (e.g., trabedersen), and small molecule inhibitors of TGF-beta (e.g., LY2157299) (see, e.g., Wojtowicz-Praga, Invest. New Drugs 21(1), 21-32 (2003)).

Another example of therapies that antagonize immunosuppression cytokines can include anti-IL-6 antibodies (e.g., siltuximab) (see, e.g., Guo et al., Cancer Treat. Rev. 38(7), 904-910 (2012). Monoclonal antibodies against IL-10 or its receptor can also be used, see, e.g., humanized versions of those described in Llorente et al., Arthritis Rheum. 43(8), 1790-1800 (2000) (anti-IL-10 mAb) or Newton et al., Clin. Exp. Immunol. 177(1), 261-268 (2014) (anti-interleukin-10R1 monoclonal antibody). Monoclonal antibodies against CCL2 or its receptors can also be used. In some embodiments, the cytokine immunotherapy is combined with a commonly used chemotherapeutic agent (e.g., gemcitabine, docetaxel, cisplatin, tamoxifen, paclitaxel, and the like) as described in U.S. Pat. No. 8,476,246.

In some embodiments, immunotherapies can include agents that are believed to elicit “danger” signals, e.g., “PAMPs” (pathogen-associated molecular patterns) or “DAMPs” (damage-associated molecular patterns) that stimulate an immune response against the cancer. See, e.g., Pradeu and Cooper, Front. Immunol. 3, 287 (2012); Escamilla-Tilch et al., Immunol. Cell. Biol. 91(10), 601-610 (2013). In some embodiments, immunotherapies can agonize toll-like receptors (TLRs) to stimulate an immune response. For example and without limitation, TLR agonists include vaccine adjuvants (e.g., 3M-052) and small molecules (e.g., imiquimod, muramyl dipeptide, CpG, and mifamurtide (muramyl tripeptide)), as well as polysaccharides such as krestin and endotoxin. See, e.g., Galluzi et al., Oncoimmunol. 1(5), 699-716 (2012); Lu et al., Clin. Cancer Res. 17(1), 67-76 (2011); and U.S. Pat. Nos. 8,795,678 and 8,790,655. In some embodiments, immunotherapies can involve administration of cytokines that elicit an anti-cancer immune response, see, e.g., Lee & Margolin, Cancers 3, 3856-3893 (2011). For example, the cytokine IL-12 can be administered (see, e.g., Portielje et al., Cancer Immunol. Immunother. 52, 133-144 (2003)) or as gene therapy (see, e.g., Melero et al., Trends Immunol. 22(3), 113-115 (2001)). In another example, interferons (IFNs), e.g., IFNy, can be administered as adjuvant therapy (see, e.g., Dunn et al., Nat. Rev. Immunol. 6, 836-848 (2006)).

In some embodiments, immunotherapies can antagonize cell surface receptors to enhance the anti-cancer immune response. For example, antagonistic monoclonal antibodies that boost the anti-cancer immune response can include antibodies that target CTLA-4 (e.g., ipilimumab, see, e.g., Tarhini and Iqbal, Onco. Targets Ther. 3, 15-25 (2010) and U.S. Pat. No. 7,741,345, or tremelimumab) or antibodies that target PD-1 (e.g., nivolumab, see, e.g., Topalian et al., New Engl. J. Med. 366 (26), 2443-2454 (2012) and Int. Pub. No. WO 2013/173223A1, pembrolizumab/MK-3475, pidilizumab (CT-011), and the like).

In other embodiments, the immunotherapies can include antibodies that target TIM-3 (T-cell immunoglobulin domain and mucin domain-3, see, e.g., Schwartz et al., Immunother Adv. 2(1), ltac019 (2022) and Zeidan et al., Exp. Rev. Anticancer Therap. 5, 523-534 (2021)). Non-limiting examples of anti-TIM3 antibodies include AZD7789 (Astra Zeneca), BGB-A425 (Beigene), BMS-986,258 (Bristol Myers Squibb), ICAGNO2390 (Incyte Corporation), LB1410 (L&L biopharma Co., Ltd.), LY3321367 (Eli Lilly and Company), R07121661 (Hoffmann-La Roche), sabatolimab (hIgG4, S228P; Novartis Pharmaceuticals, developed as MBG453), SHR-1702 (Jiangsu HengRui Medicine), Sym023 (Symphogen AS), TSR-022 (cobolimab; Tesaro), and the like.

In yet other embodiments, the immunotherapies can include antibodies that target LAG3 (Lymphocyte-Activation Gene 3, see, e.g., Chocarro et al., Cells 11(15), 2351 (2022) and Tian et al., Exp. Rev. Anticancer Therap. 3, 289-296 (2022)). Non-limiting examples of anti-LAG3 antibodies include relatimab (Bristol Myers Squibb, developed as BMS-986016), opdualag (Bristol Myers Squibb), GSK2831781 (GlaxoSmithKline), HLX26 (Fosun Pharma), IBI110 (Innovent Biologics), INCAGNO2385 (Incyte), LAG525 or IMP701 (Novartis), MK-4830 or favezelimab (Merck), REGN3767 or fianlimab (Regeneron Pharmaceuticals and Sanofi), Sym022 (Symphogen), TSR-033 (Tesaro), ABL501 (ABL Bio), CB213 Humabody® (Crescendo Biologics), EMB-02 (EpimAb Biotherapeutics), EOC202 (Taizhou EOC Pharma), FS 118 (F-star Therapeutics), IBI323 (Innovent Biologic), IMP321 or eftilagimod alpha or efti (Immutep), MGDO13 or tebotelimab (MacroGenics), R07247669 (Hoffmann-LaRoche), XmAb®22841 or pavunalimab (Xencor), and the like.

Some immunotherapies enhance T cell recruitment to the tumor site (e.g., such as endothelin receptor-A/B (ETRA/B) blockade, e.g., with macitentan or the combination of the ETRA and ETRB antagonists BQ123 and BQ788, see, e.g., Coffman et al., Cancer Biol. Ther. 14(2), 184-192 (2013)) or enhance CD8 T-cell memory cell formation (e.g., using rapamycin and metformin, see, e.g., Pearce et al., Nature 460(7251), 103-107 (2009); Mineharu et al., Mol. Cancer Ther. 13(12): 3024-3036 (2014); and Berezhnoy et al., Oncoimmunology 3, e28811 (2014)). Immunotherapies can also include administering one or more of: adoptive cell transfer (ACT) involving transfer of ex vivo expanded autologous or allogeneic tumor-reactive lymphocytes, e.g., dendritic cells or peptides with adjuvant; cancer vaccines, such as DNA-based vaccines, cytokines (e.g., IL-2), cyclophosphamide, anti-interleukin-2R immunotoxins, and/or prostaglandin E2 inhibitors (e.g., using SC-50). In some embodiments, the methods include administering a composition comprising tumor-pulsed dendritic cells, e.g., as described in Int. Put. No. WO 2009/114547 and references cited therein. See also Shiao et al., Genes Dev. 25: 2559-2572 (2011).

Yet other examples of immunotherapies include, but are not limited to, adoptive T cell therapies or cancer vaccine preparations designed to induce T lymphocytes to recognize cancer cells, as well as checkpoint inhibitors such as anti-CD137 (e.g., BNA035 (Binacea Pharma), BMS-663513 (Bristol Myers Squibb), FS222 (F-Star Therapeutics), FS120 (F-Star Therapeutics), MCLA-145 (Merus N.V./Incyte), PRS-343 (Pieris Pharmaceuticals), PRS-344/S095012 (Pieris Pharmaceuticals/Institut de Recherches Internationales Servier), and the like), anti-PD1 (e.g., nivolumab, pembrolizumab/MK-3475, pidilizumab (CT-011), and the like), anti-PDL1 (e.g., BMS-936559, MPDL3280A, and the like), or anti-CTLA-4 (e.g., ipilimumab, tremelimumab, and the like; see, e.g., Kruger et al., Histol. Histopathol. 22(6), 687-696 (2007); Eggermont et al., Semin. Oncol. 37(5), 455-459 (2010); Klinke, Mol. Cancer. 9, 242 (2010); Alexandrescu et al., J. Immunother. 33(6), 570-590 (2010); Moschella et al., Ann. N.Y. Acad. Sci. 1194, 169-178 (2010); Ganesan and Bakhshi, Nat'l Med. J. India 23(1), 21-27 (2010); and Golovina and Vonderheide, Cancer J. 16(4), 342-347 (2010)).

In another instance, the combination therapy can include anti-angiogenesis therapy, and the method can further include administering a therapeutically effect amount of at least one angiogenesis inhibitor to the subject. In some embodiments, the angiogenesis inhibitor is an anti-VEGF antibody, an anti-VEGF receptor antibody, a VEGF receptor kinase inhibitor, an anti-FGF antibody, an anti-FGF receptor antibody, an FGF receptor kinase inhibitor, an anti-PDGF antibody, an anti-PDGF receptor antibody, a PDGF receptor kinase inhibitor, an anti-EGF antibody, an anti-EGF receptor antibody, or an EGF receptor kinase inhibitor. Administration of the angiogenesis inhibitor can occur at a timepoint before, after, or during the administration of the composition, at least one immune checkpoint inhibitor, and/or radiation, or other therapies described herein.

In particular embodiments, the method can include administering a therapeutically effective amount of at least one VEGF inhibitor (e.g., an anti-VEGF antibody, an anti-VEGFR2 antibody, and the like) at a timepoint before, after, or during the administration of the composition, at least one immune checkpoint inhibitor, and/or radiation, or other therapies described herein.

Yet other non-limiting angiogenesis inhibitors can include one or more of the following: VEGF (vascular endothelial growth factor) inhibitors (e.g., VEGF receptor kinase inhibitor, anti-VEGF receptor antibody, and anti-VEGF antibody, see, e.g., Asano et al., Cancer Res. 55, 5296-5301 (1995), as well as bevacizumab (anti-VEGF monoclonal antibody), 2C3 antibody, IMC-1121b, IMC-18F1, IMC-1C11, and IMC-2C6); FGF (fibroblast growth factor) inhibitors (e.g., FGF receptor kinase inhibitor, anti-FGF receptor antibody, and anti-FGF antibody, see, e.g., Hori et al., Cancer Res. 51, 6180-6184 (1991)); PDGF (platelet-derived growth factor) inhibitors (e.g., PDGF receptor kinase inhibitor (see, e.g., Bergers et al., J. Clin. Invest. 111, 1287-1295 (2003)), anti-PDGF receptor antibody, anti-PDGF antibody); EGF (epidermal growth factor) inhibitors (e.g., EGF receptor kinase inhibitor (Hori et al., Cancer Res. 51, 6180-6184 (1991)), anti-EGF receptor antibody, and anti-EGF antibody); integrin inhibitors (e.g., avP3 integrin inhibitor, avP5 integrin inhibitor (see, e.g., Gutheil et al., Clin. Cancer Res. 6, 3056-3061 (2000)); endogenous inhibitors (e.g., IL-12, thrombospondin-1, endostatin, angiostatin (see, e.g., Dias et al., Int. J. Cancer 78, 361-365 (1998)), COX-2 inhibitor (see, e.g., Masferrer et al., Ann. N.Y. Acad. Sci. 889, 84-86 (1999))); matrix metalloprotein inhibitors (see, e.g., Bramhall, Int. J. Pancreatol. 21, 1-12 (1997)); other inhibitors (e.g., farnesyltransferase inhibitor, nitric oxide inhibitor, angiotensin-converting enzyme inhibitor, HMG-CoA reductase inhibitor, vascular target inhibitor, methionine aminopeptidase inhibitor (see, e.g., Liu et al., Science, 282, 1324-1327 (1998))); and so on.

Yet other angiogenesis inhibitors can include a compound or molecule including, but not limited to, peptides, proteins, enzymes, polysaccharides, oligonucleotides, DNA, RNA, recombinant vectors, and drugs that function to inhibit angiogenesis. Angiogenesis inhibitors are known in the art and all types are contemplated herein. Yet other non-limiting examples of compounds and molecules include natural and synthetic biomolecules such as paclitaxel, O-(chloroacetyl-carbomyl) fumagillol (TNP-470 or AGM 1470), thrombospondin-1, thrombospondin-2, angiostatin, human chondrocyte-derived inhibitor of angiogenesis (hCHIAMP), cartilage-derived angiogenic inhibitor, platelet factor-4, GRO (growth-regulated oncogene)-beta, human interferon-inducible protein 10 (IP10), interleukin 12, Ro 318220, tricyclodecan-9-yl xanthate (D609), irsogladine, 8,9-dihydroxy-7-methyl-benzo[b]quinolizinium bromide (GPA 1734), medroxyprogesterone, a combination of heparin and cortisone, glucosidase inhibitors, genistein, thalidomide, diamino-antraquinone, herbimycin, ursolic acid, and oleanolic acid. Non-limiting examples of antibodies include those directed towards molecules such as VEGF, VEGF receptor, or different epitopes of endoglin. Additionally, small molecular inhibitors of VEGF receptor are known and contemplated herein. Non-limiting examples of VEGF receptor inhibitors include ranibizumab (Lucentis), aflibercept (VEGF-Trap), sunitinib (Sutent), sorafenib (Nexavar), axitinib, pegaptanib, and pazopanib.

For any antibodies described herein, variants can be included, such as monoclonal, polyclonal, bi-specific, IgG1, or IgG4 forms of any of these.

In yet another instance, the combination therapy can include radiotherapy, and the method can further include administering a therapeutically effect amount of radiation to the subject. In some embodiments, the radiation includes x-rays, gamma rays, electron beam radiation, proton beam radiation, or ionizing particles. Administration of radiation inhibitor can occur at a timepoint before, after, or during the administration of the composition, at least one immune checkpoint inhibitor, and/or at least one anti-angiogenesis inhibitor, or other therapies described herein.

Use of the compositions herein with a combination therapy can be employed to treat a cancer. In some embodiments, the cancer is selected from liver cancer, lung cancer, prostate cancer, breast cancer, glioblastoma, melanoma, pancreatic cancer, colorectal cancer, and leukemia. Other cancers include any described herein, e.g., in Table 1.

In particular embodiments, combination therapy is employed with a composition for treating p53-deficient cancer cells. In some embodiments, the composition can include a p53-encoding mRNA within a delivery vehicle capable of providing release of the p53-encoding mRNA in a cancer cell. In some embodiments, the delivery vehicle is a particle (e.g., a nanoparticle) including the p53-encoding mRNA and a complexing agent (e.g., a lipid-like compound) within a core. In particular embodiments, the particle further includes a shell, which in turn can include at least one amphiphilic material disposed around the core. Any useful amphiphilic material can be employed, such as, e.g., a group consisting of lecithin, a phospholipid (e.g., phosphatidic acid, phosphatidyl choline, phosphatidyl ethanolamine, phosphatidylglycerol, phosphatidylserine, phosphatidylinositol, lysophosphatidyl, cardiolipin, or β-acyl-y-alkyl phospholipid), and/or a pegylated lipid (e.g., ceramide-PEG and/or 2-distearoyl-sn-glycero-3-phosphoethanolamine (DSPE)-terminated PEG).

In some embodiments, the complexing agent is selected from a group consisting of a cationic lipid-like compound and a cationic lipid. In other embodiments, the cationic lipid-like compound is Gₘ-Cₙ, wherein m≥0 and n<20; and the cationic lipid is Lipofectamine™ 2000, 2,3-dioleyloxy-N-[2-(sperminecarboxamido)ethyl]-N,N-dimethyl-1-propanaminium (DOSPA, including salts thereof, such as HCl salt, a trifluoroacetate salt, chloride salt, and the like), 1,2-dioleoyl-sn-glycerophosphoethanolamine (DOPE), a combination thereof, or any other described herein.

In non-limiting embodiments, the core can further include a water-insoluble polymer (e.g., poly(lactic-co-glycolic acid) (PLGA), poly(lactic acid) (PLA), and poly(glycolic acid) (PGA); an amphiphilic polymer; a copolymer of polyethylene glycol (PEG) and a polyester selected from PLGA, PLA, or PGA; or any other described herein). In other embodiments, the core lacks a water-insoluble polymer.

### EXAMPLES

### Example 1: Combining p53 mRNA Nanotherapy with Immune Checkpoint Blockade Reprograms the Immune Microenvironment for Effective Cancer Therapy

Loss of function in tumor suppressors can be a driving force in tumorigenesis and the development of therapeutic resistance. The p53 tumor suppressor gene, a master regulator of cell cycle arrest, apoptosis, senescence, and other cellular pathways,¹ is frequently mutated in a myriad of human cancers, including hepatocellular carcinoma (HCC). Beyond cell autonomous tumor-suppressive effects, increasing evidence indicates that p53 protein can also regulate the immune tumor microenvironment (TME) by modulating interactions of tumor cells with immune cells.²⁻⁶ For example, p53 has been shown to induce antitumor immune response via transcriptional regulation of genes encoding for key cytokines (e.g., TNF-α, IL-12, and IL-15),⁷⁻⁹ chemokines (e.g., CCL2, -20, and -28, and CXCL1, -2, -3, -5, and -8)₁°-” and pathogen recognition (e.g., Toll-like receptors, TLRs),¹²⁻¹³ all of which result in recruitment and activation of immune cells. Genetic restoration of p53 could induce the activation of myeloid cells to promote tumor antigen-specific adaptive immunity¹⁴ and upregulate the NKG2D ligands on senescent tumor cells for activation of natural killer (NK) cells.” p53 may also play an important role in the suppression of pro-tumorigenic M2-type tumor-associated macrophage (TAM) polarization, thus facilitating antitumor immunity.¹⁶⁻¹⁷ Moreover, recent studies suggest that immunogenic cancer cell death induced by cytotoxic agents may be associated with activation of the p53 pathway.¹⁸⁻¹⁹ Despite these advances in understanding the role of p53, developing therapeutic approaches that directly and effectively address the loss of p53 function and its role in immunosuppression and immunotherapy resistance in HCC remains an elusive goal.

HCC is the most prevalent liver cancer with a high mortality rate and dismal prognosis.²⁰⁻²² Enhancing anti-tumor immunity using immune checkpoint blockade (ICB), including anti-CTLA-4, anti-PD-1 (aPDI), and anti-PD-L1 (aPD-L1) antibodies, has demonstrated the potential to transform the therapeutic landscape of many cancers including HCC. However, responses are typically seen in a limited fraction of patients, and majority of cancer patients do not benefit from the treatment. Without wishing to be limited by mechanism, this may be mediated in part by insufficient tumor immunogenicity and the immunosuppressive TME. Different strategies are actively being developed to improve ICB therapy in HCC, with a major focus on combining ICB with other existing therapies (such as anti-VEGF therapy), which could significantly increase anti-tumor immunity. Such combinations have been shown to improve anti-tumor efficacy in animal models and increase the survival of patients in clinical trials.²³⁻²⁶ However, an increasing majority of HCC patients show no responses, and thus, new combinatorial strategies are still desperately needed.

Accordingly, immunotherapy with ICB has shown limited benefits in hepatocellular HCC other cancers, mediated in part by the immunosuppressive TME. As p53 loss of function may play a role in immunosuppression, we herein examine the effects of restoring p53 expression on the immune TME and ICB efficacy.

As described herein, we address the unmet need to implement p53 therapy and potentiate ICB response in HCC. We report a targeted mRNA nanoparticle (NP) platform designed to induce p53 expression and reprogram the TME, which we test in proof-of-concept studies in combination with ICB inp53-null murine HCC models. We optimize the p53 mRNA NP platform for HCC targeting, evaluate its therapeutic efficacy inp53-null HCCs growing in orthotopic and ectopic sites (alone or with aPDI antibody), and study changes in the TME. This combinatorial strategy safely and effectively inhibits tumor growth in vivo, while prolonging survival and reducing ascites and metastases. Thus, combining p53 mRNA nanotherapy with ICB immunotherapy could become a transformative approach for the treatment of HCC and potentially other cancers involving p53 deficiency.

In particular, we have developed and optimized a CXCR4-targeted mRNA NP platform to effectively induce p53 expression in HCC models. Using p53-null orthotopic and ectopic models of murine HCC, we find that combining CXCR4-targeted p53 mRNA nanoparticles with anti-PD-1 therapy effectively induces global reprogramming of cellular and molecular components of the immune TME. This effect results in improved anti-tumor effects, as compared to anti-PD-1 therapy alone or therapeutic p53 expression alone. Thus, our findings demonstrate the reversal of immunosuppression in HCC by a p53 mRNA nanomedicine when combined with ICB and support the implementation of this strategy for cancer treatment. Additional details follow.

### Example 2: Non-Limiting Experimental Methods

### Materials

Ester-terminated PLGA (with inherent viscosity of 0.55-0.75 dL/g) was purchased from Durect Corporation. Lipid PEGs terminated with methoxyl groups (1,2-distearoyl-sn-glycero-3-phosphoethanolamine-N-[methoxy(polyethylene glycol)-3000](ammonium salt), DSPE-MPEG (molecular weight (MW) of PEG, 3000 Da) were purchased from Avanti Polar Lipids. Cationic ethylenediamine core-poly(amidoamine) (PAMAM) dendrimer generation 0 (Go) were purchased from Sigma-Aldrich. CXCR4-targeting peptide CTCE-9908 (KGVSLSYRCRYSLSVGK, CTCE, SEQ ID NO: 1) and scrambled peptide (LYSVKRSGCGSRKVSYL, SCP, SEQ ID NO: 2) were custom synthesized by GL Biochem (Shanghai) Ltd. Lipofectamine 2000 (L2K) was purchased from Invitrogen. Firefly Luciferase mRNA (Luc mRNA, L-7202), Enhanced Green Fluorescent Protein mRNA (EGFP mRNA, L-7201), and Cyanine 5 Firefly Luciferase mRNA (Cy5-Luc mRNA, L-7702) were purchased from TriLink Biotechnologies (San Diego, CA). Murine p53 mRNA with chemical modification (full substitution of Pseudo-U and 5-Methyl-C, Capped (Cap 1) using CleanCap® AG, Polyadenylated (120 A)) was custom-synthesized by TriLink Biotechnologies (San Diego, CA). InVivoMAb anti-mouse PD-1 (CD279) was purchased from Bioxcell. D-luciferin-K+salt bioluminescent substrate (no. 122799) was obtained from PerkinElmer. Primary antibodies used for western blot experiments as well as immunofluorescent and immunohistochemistry staining included: anti-p53 (sc-126, Santa Cruz Biotechnology, 1:500 dilution), anti-GAPDH (Cell Signaling Technology, #5174; 1:2000 dilution), anti-beta-Actin (Cell Signaling Technology; 1: 2,000 dilution), and anti-rabbit and anti-mouse horseradish peroxidase (HRP)-conjugated secondary antibodies (Cell Signaling Technology). Secondary antibodies used in this study included: Alexa Fluor® 488 Goat-anti Rabbit IgG (Life Technologies, A-11034), and Alexa Fluor® 647 Goat-anti Mouse IgG (Life Technologies, A-28181). All other chemicals and solvents were purchased from Sigma-Aldrich and used without further purification.

Synthesis of Ionizable Lipid-Like Compounds (G₀-Cₙ)

A series of ionizable lipid-like compounds termed G₀-Cₙ were synthesized through ring opening of epoxides bearing different alkyl chain lengths by generation 0 of poly (amidoamine) (PAMAM) dendrimers (M1). Briefly, substoichiometric amounts of epoxide were added to increase the proportion of products with one less tail than the total possible for a given amine monomer. The amine (1 equiv., typically 1 millimole (mmol)) and epoxide (9 equiv., typically 1 millimole (mmol)) were added to a 50 mL round-bottom glass flask containing a magnetic stir bar. The flask was sealed, and the reaction was heated to 95° C. with homogeneous stirring for 2 days. The crude products were separated by chromatography on silica with gradient elution from CH₂Cl₂ to 15:1 CH₂Cl₂/MeOH. The separated product was characterized by ¹H-NMR spectrum.

mRNA Complexation Ability of G₀-C₈ and its Stability in Organic Solvent

Gel electrophoresis was used to study the mRNA complexation ability of ionizable compound G₀-C₈ and optimize the ratio between G₀-C₈ and mRNA in the NPs with free EGFP-mRNA or EGFP-mRNA complexed with G₀-C₈. Free EGFP-mRNA was also incubated with DMF to evaluate the stability of mRNA in organic solvent (DMF). The EGFP-mRNA were first incubated with G₀-C₈ at different weight ratios (weight ratios of G₀-C₈/mRNA: 1, 2, 5, 10, and 20) or DMF for 20 min at room temperature. The volumes of samples were then adjusted with loading dye (Invitrogen) and run into an E-Gel 2% agarose (Invitrogen) gel for 30 min at 50 V. Ambion Millennium markers-Formamide (Thermo Fisher Scientific) was used as a ladder. Finally, the gel was imaged under ultraviolet and the bands were analyzed.

Synthesis of Lipid-PEG-CTCE HCC Targeting Peptide (DSPE-PEG-CTCE) and Lipid-PEG-Scrambled peptide (DSPE-PEG-SCP)

We conjugated the CXCR4-targeting peptide CTCE-9908 (KGVSLSYRCRYSLSVGK, CTCE, SEQ ID NO: 1) and scrambled peptide (LYSVKRSGCGSRKVSYL, SCP, SEQ ID NO: 2) to DSPE-PEG-MAL to construct the HCC targeted NPs and the non-targeted control NPs, respectively. Synthesis of DSPE-PEG-CTCE and DSPE-PEG-SCP was achieved through the efficient thiol-maleimide Michael addition click reaction. In brief, DSPE-PEG-maleimide and the thiol-CTCE peptide (3:1) or thiol-scrambled peptide were each dissolved in dimethylsulfoxide or dimethylformamide (DMF). The peptide solution was diluted in 0.1 M sodium phosphate buffer, pH 7.4; and DSPE-PEG was then added to the mixture. The final reaction mixture was 1:1 DMF/(sodium phosphate buffer) with 5 mM peptide and 15 mM DSPE-PEG maleimide. The reaction was allowed to proceed for 2 h at room temperature and then dialyzed against DI water for purification. Lastly, the product was lyophilized to obtain white powder as the final product (DSPE-PEG-CTCE or DSPE-PEG-SCP). The chemical structures of DSPE-PEG-CTCE and DSPE-PEG-SCP were confirmed by ¹H-NMR spectrum.

Optimization of the mRNA NPs: The Effect of Targeting Ligand Densities

The cellular uptake of Enhanced Green Fluorescent Protein mRNA (EGFP mRNA) NPs engineered with seven different densities of CTCE peptide (EGFP-mRNA-CTCE NPs, CTCE density: 2%, 3%, 4%, 5%, 6%, 7%, and 10%, respectively) and 5% scrambled peptide (SCP) was studied to optimize the surface chemistry and targeting efficacy of the mRNA NPs by measuring GFP expression using flow cytometry (BD Biosystems, Heidelberg, Germany) and analyzed using Flowjo software (Flowjo V10).

Preparation of mRNA NPs and the Formulation Optimization

An optimized and robust self-assembly technique was employed to prepare mRNA-encapsulated polymer-lipid hybrid NPs based on our previous report,²⁷ but we extensively optimized the ratios among different NPs' components, the pH of the solution for mRNA complexation, and the sequence in which reagents were added, which affected the encapsulation, morphology, and transfection efficiency of the mRNA. Briefly, G₀-C₈ and PLGA were dissolved separately in anhydrous DMF to form a homogeneous solution at concentrations of 2.5 mg/ml and 5 mg/ml, respectively. DSPE-MPEG, DSPE-PEG-CTCE and DSPE-PEG-SCP were dissolved in DNase/RNase-free HyPure water (GE Healthcare Life Sciences, catalog no. SH30538) at the concentration of 1 mg/mL. All of the reagents listed above were sonicated for 5 min in a water-bath sonicator before use. Citrate buffer with pH 3.0-3.5 was first added to 80 μg of G₀-C₈ (in 32 μl of DMF), then 16 μg of p53 mRNA (in 16 μl of citrate buffer) was added, mixed gently (at a G₀-C₈/mRNA weight ratio of 5), and allowed to stay at room temperature for 15 min to ensure the sufficient electrostatic complexation. Afterwards, 250 g of PLGA polymers (in 50 μl of DMF) was added to the mixture and gently mixed. The final mixture was added dropwise to 10 ml of DNase/RNase-free HyPure water consisting of 1 mg hybrid lipid-PEGs under uniform magnetic stirring (1000 rpm) for 30 min. An ultrafiltration device (EMD Millipore, MWCO 100 kDa) was used to remove the organic solvent and free compounds from the NP dispersion via centrifugation at 4° C. After washing three times with DNase/RNase-free HyPure water, the mRNA NPs were collected and finally concentrated in pH 7.4 PBS buffer. The NPs were used fresh or stored at −80° C. for further use.

Physicochemical Characterization and Stability of mRNA NPs

The hydrodynamic diameter, zeta potential, and morphology of the p53-mRNA NPs were measured to assess their physicochemical properties. Sizes and zeta potentials of both CTCE- p53-mRNA NPs and SCP-p53-mRNA NPs were measured by dynamic light scattering (DLS, Brookhaven Instruments Corporation) at 20° C. Diameters are reported as the intensity mean peak average. To prepare NPs for Transmission Electron Microscopy (TEM) to characterize their morphology and shape, CTCE-p53-mRNA NPs were negatively stained with 2% uranyl acetate and then imaged with a Tecnai G₂ Spirit BioTWIN microscope (FEI Company). To verify the in vitro stability of the synthesized polymer-lipid hybrid mRNA NPs in an environment mimicking the physiological milieu, CTCE-p53-mRNA NPs were incubated in 10% serum-containing PBS solution at 37° C. in triplicate for 96 hr with constant stirring at 100 rpm. At each time point, an aliquot of NP solution was withdrawn for particle size measurement using DLS and analyzed at various time intervals to evaluate any change in size distribution. To test the encapsulation efficiency (EE %) of mRNA in the NPs, Cy5-Luc-mRNA NPs were prepared according to the aforementioned method. Dimethyl sulfoxide (DMSO, 100 l) was added to 5 μl of the NP solution to extract the mRNA encapsulated in the NPs, and the fluorescence intensity of Cy5-Luc-mRNA was measured using a multi-mode microplate reader (TECAN, Infinite M200 Pro). The amount of loaded mRNA in the engineered NPs was calculated to be ˜67.5%.

### Cell Culture

Thep53-null murine HCC cell line RIL-175 was used throughout. RIL-175 (ap53-null/Hras mutant line syngeneic to C57B1/6 mouse strain background, Luciferase-tagged) was kindly provided by Dr. Tim Greten (NIH). All other cells were purchased from American Type Culture Collection (ATCC). Dulbecco's Modified Eagle's Medium (DMEM; ATCC) was used to culture RIL-175 cells. The cell culture medium was supplemented with 10% fetal bovine serum (Hyclone, SH30071.03), Pen-Strep (100 U ml⁻¹ and 100 g ml⁻¹, respectively). Cell culture and all biological experiments were performed at 37° C. in 5% CO₂ conditions and the normal level of O₂ in a cell culture incubator. All cell lines were routinely tested using a mycoplasma contamination kit (R&D Systems) before any in vitro cell experiments or in vivo tumor model preparation.

### Cell Viability and Transfection Efficiency of EGFP-mRNA NPs

CTCE-EGFP-mRNA NPs and SCP-EGFP-mRNA NPs were prepared for evaluated the cell viability of the mRNA NPs along with their transfection efficiency of EGFP-mRNA. For the cell viability tests, RIL-175 cells were plated in a 96-well plate at a density of 5×10³ cells per well. After 24 h of cell adherence, cells were treated with EGFP-mRNA at various mRNA concentrations (0.0625, 0.125, 0.250, 0.500, and 0.750 μg ml⁻¹) for 24 hr, the cells were washed with PBS buffer (pH 7.4), followed by changing the culture medium to 0.1 ml fresh complete medium per well and further incubation for another 24 hr to evaluate cell viability by the Alamar Blue assay according to the manufacturer's protocol and a microplate reader (TECAN, Infinite M200 Pro). To test the transfection efficiency, RIL-175 cells were seeded at a density of 5×10⁴ cells per well on a 6-well plate and allowed to attach and grow until ˜80% confluence. Cells were transfected with EGFP-mRNA NPs at the mRNA concentration of 0.5 g ml⁻¹ for 24 h, followed by washing with fresh complete medium and further incubated for 24 h to assess transfection efficiency by measuring GFP expression using flow cytometry (DXP11 Flow Cytometry Analyzer). The percentages of GFP-positive cells were calculated and analyzed using Flowjo software (Flowjo V10).

### Establishment of CXCR4-KO RIL-175 Cells

The precise gene-editing system of CRISPR (clustered regularly interspaced short palindromic repeat)/Cas9 (CRISPR associated) was performed to knock out the CXCR4 gene in RIL-175 cells. Briefly, the single guide RNA (sgRNA) targeting CXCR4 was designed on the online tool (genome-engineering.org) including sgRNA1 (forward: 5′-CACCGTCGAGAGCATCGTGCACAAG-3′ (SEQ ID NO: 3), reverse: 5′-AAACCTTGTGCACGATGCTCTCGAC-3′ (SEQ ID NO: 4)) and sgRNA 2 (forward: 5′-CACCGGGACTTACACTCACACTGAT-3′ (SEQ ID NO: 5), reverse: 5′-AAACATCAGTGTGAGTGTAAGTCCC-3′ (SEQ ID NO: 6)), and sequentially were phosphorylated and annealed. At one time, the lentiviral expression lentiCRISPRv2 plasmid (Addgene, cat. no. 52961, USA) was digested and dephosphorylated with BsmBI enzyme (ThermoFisher, cat. No. ER0451) following by running DNA gel and gel purify the larger band leaving the 2 kb filler piece. Next, the ligation reaction of lentiCRISPRv2 and sgRNAs was established for incubating 10 min at room temperature. After finishing the process of transformation in Stbl3 bacteria and validation by DNA sequencing, the lentiCRISPv2 inserted with sgRNAs targeting CXCR4 was selected out. Then, the lentivirus system including lentiCRRISPv2 and the packaging plasmids pVSVg (AddGene, cat. No. 8454) and psPAX2 (AddGene, Cat. No. 12260) were co-transfected into HEK293T cells to produce the complete lentivirus and further transfected into RIL-175 wide type cells. The puromycin (2 μg/μl) previously included in the lentiCRISPRv2 was used to screen out the positive cells successfully transfected with the complete lentivirus. Finally, the quantitative PCR and western blotting were performed to detect the expression of CXCR4 from both transcriptional and protein levels.

Cellular Uptake of Dye-Labeled mRNA-Encapsulated NPs

To monitor the cellular uptake of the NPs, Cy5-Luc-mRNA-NPs were prepared. RIL-175 cells were first seeded in 35 mm confocal dishes (MatTek) at a density of 5×10⁴ cells per well and incubated at 37° C. in 5% CO₂ for 24 h. The cells were then incubated with medium (DMEM) containing Cy5-Luc-mRNA-NPs at different time intervals. The cells were then washed with PBS, counterstained with Hoechst 33342 (Thermofisher), and analyzed using an Olympus microscope (FV1200, Olympus).

In Vitro Cell Growth Inhibition Assay with p53-mRNA NPs

RIL-175 or HCA-1 cells were plated in 96-well plates at a density of 5×10³ cells per well. After 24 h of cell adherence, cells were treated with empty NPs (blank NPs), free p53 mRNA, or p53-mRNA NPs at different mRNA concentrations (0.0625, 0.125, 0.250, 0.500, and 0.750 μg ml⁻¹). After 24 h of incubation, the cells were washed with PBS buffer (pH 7.4) and further incubated in fresh medium for another 24 h. AlamarBlue cell viability was used to verify the in vitro cell growth inhibition efficacy of p53-mRNA NPs.

### Immunoblotting

Protein extracts from cells taken from dissected tumors in each group were prepared using lysis buffer (1 mM EDTA, 20 mM Tris-HCl pH 7.6, 140 mM NaCl, 1% aprotinin, 1% NP-40, 1 mM phenylmethylsulphonyl fluoride, and 1 mM sodium vanadate), and supplemented with protease inhibitor cocktail (Cell Signaling Technology) and boiled at 100° C. for 10 min. Equal amounts of protein were determined with a bicinchoninic acid protein assay kit (Pierce/Thermo Scientific) according to the manufacturer's instructions. After gel electrophoresis and protein transformation, membranes were blocked with 3% bovine serum albumin (BSA) in TBST (150 mM NaCl, 50 mM Tris-HCl at pH 7.4, and 0.1% Tween 20) for 1 h at room temperature with gentle shaking. Membranes were rinsed and then incubated overnight at 4° C. with appropriate primary antibodies. The immunoreactive bands were visualized using an enhanced chemiluminescence (ECL) detection system (Cell Signaling Technology).

Immunofluorescence staining and microscopy

For immunofluorescence staining, cells or tumor tissues from each treatment group were washed with ice-cold PBS and fixed with 4% paraformaldehyde (Electron Microscopy Sciences) in PBS for 20 min at room temperature, followed by permeabilization in 0.2% Triton X-100-PBS for 10 min. Samples were followed by blocking with PBS blocking buffer containing 2% normal goat serum, 2% BSA, and 0.2% gelatin for 1 h at room temperature. Then, the samples were incubated in primary antibodies at the appropriate concentration for 1 h at room temperature, washed with PBS and incubated in goat anti-rat-Alexa Fluor® 647 (Molecular Probes) at 1:1000 dilution in blocking buffer for another 1 h at room temperature. Finally, stained cells were washed with PBS, counterstained with Hoechst 33342 (Molecular Probes-Invitrogen, H1399, 1:10000 dilution in PBS), and mounted on slides with Prolong Gold antifade mounting medium (Life Technologies). The slides were imaged under a confocal laser scanning microscope (Olympus, FV1100).

### Animals

For the s.c. tumor model, all animal procedures were performed in ethical compliance and with approval by the Institutional Animal Care and Use Committees at Harvard Medical School. Immunocompetent male and female C57BL/6 mice (5-6 weeks old or 6-8 weeks old) were obtained from Charles River Laboratories and housed in a pathogen-free animal facility of Brigham and Women's Hospital, Harvard Medical School. For each experiment, mice were randomly allocated to each group. Mice were put for at least a 72 h acclimation period prior to use in order for physiological parameters to return to baseline after shipping and transferring. All animals were housed in single-unit cages with 12-h alternate light and dark cycles and at controlled ambient temperature (68-79° F.) with humidity between 30%-70%. For the orthotopic tumor model, all animal experiments were performed after approval by the Institutional Animal Care and Use Committee of the Massachusetts General Hospital.

### Pharmacokinetics Study

Healthy C57B1/6 mice (5-6 weeks old, n=3 per group) were injected intravenously with free Cy5-Luc-mRNA, CTCE-Cy5-Luc-mRNA NPs, or SCP-Cy5-Luc-mRNA NPs through the tail vein at the mRNA dose of 350 g per kg of animal weight. Blood was collected retroorbitally at different time points (5 min, 30 min, 1 h, 2 h, 6 h, 12 h, and 24 h); and the fluorescence intensity of Cy5-Luc-mRNA was measured using a microplate reader (TECAN, Infinite M200 Pro). Pharmacokinetics was evaluated by calculating the percentage of Cy5-Luc mRNA in blood at various time points.

### HCC Tumor Model Preparation

Two p53-null RIL-175 HCC tumor models, an ectopic (s.c.) grafted model and an orthotopic model, were developed for in vivo biodistribution, modulation of the immune microenvironment, therapeutic efficacy, and in vivo toxicity studies. An orthotopic p53-wild type HCA-1 HCC tumor model was also developed for the in vivo therapeutic efficacy study. For the s.c. grafted model, ˜1×10⁶ RIL-175 cells in 100 μl of culture medium mixed with 100 μl of matrigel (BD Biosciences) were implanted subcutaneously in the right flank of C57B1/6 mice (6-8 weeks old). Mice were monitored for tumor growth every other day according to the animal protocol. To develop the RIL-175 orthotopic model, ˜1 million RIL-175 cells 1:1 in Matrigel (Mediatech/Corning, Manassas, VA) were grafted into the left extrahepatic lobe of C57B1/6 mice (6-8 weeks old). Tumor growth was monitored by high-frequency ultrasonography every 3 days according to the animal protocol. For the HCA-1 orthotopic model, approximately 1 million HCA-1 cells 1:1 in Matrigel (Mediatech/Corning, Manassas, VA) were grafted into the left extrahepatic lobe of C₃H mice (6-8 weeks old). Tumor growth was monitored by high-frequency ultrasonography every 3 days according to the animal protocol. When the tumor volume reached about ˜100 mm³ (for ectopic model) or ˜5 mm in diameter (for orthotopic model), mice were randomly assigned to a treatment group.

Biodistribution of mRNA NPs in the RIL-175 HCC Tumor Model

The biodistribution and tumor accumulation of mRNA NPs were assessed in C57B1/6 mice bearing with s.c. grafted RIL-175 tumor (˜100-200 mm³) and in the RIL-175 orthotopic model (˜5 mm in diameter), respectively. In brief, RIL-175 bearing C57B1/6 mice (5-6 weeks old, n=3 per group) were injected intravenously with free Cy5-Luc-mRNA, CTCE-Cy5-Luc NPs, or SCP-Cy5-Luc NPs via the tail vein at a mRNA dose of 350 μg per kg of animal weight. After 24 h, all the mice were sacrificed, and dissected organs and tumors were visualized using a Syngene PXi imaging system (Synoptics Ltd). The data were analyzed by Image J software.

### Flow Cytometry and Cytokine Analysis

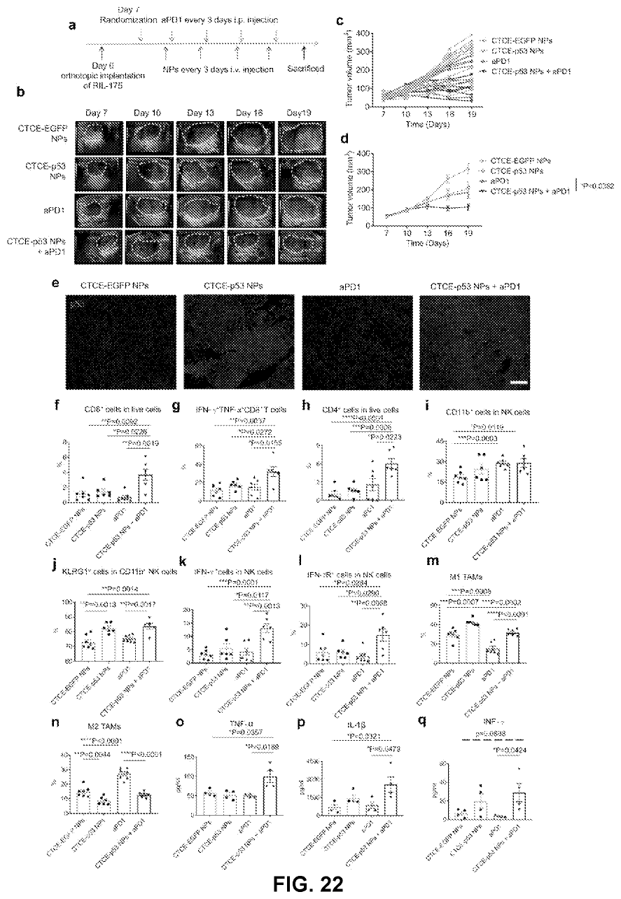

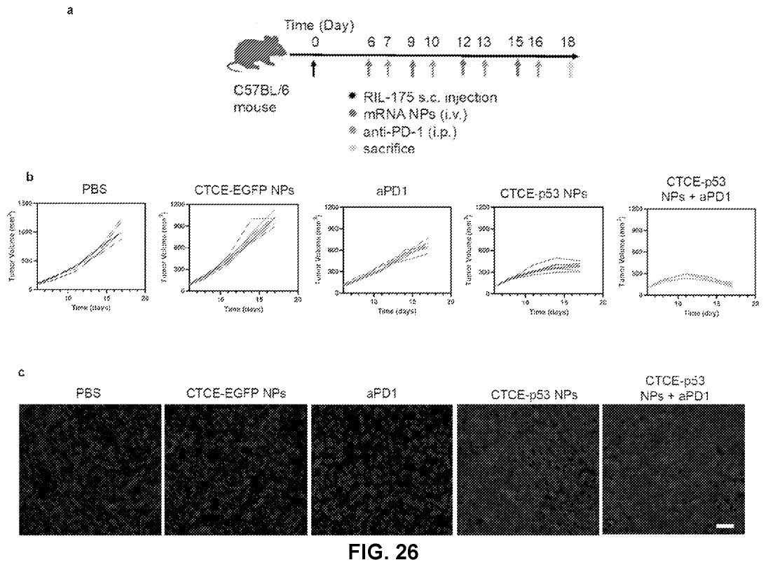

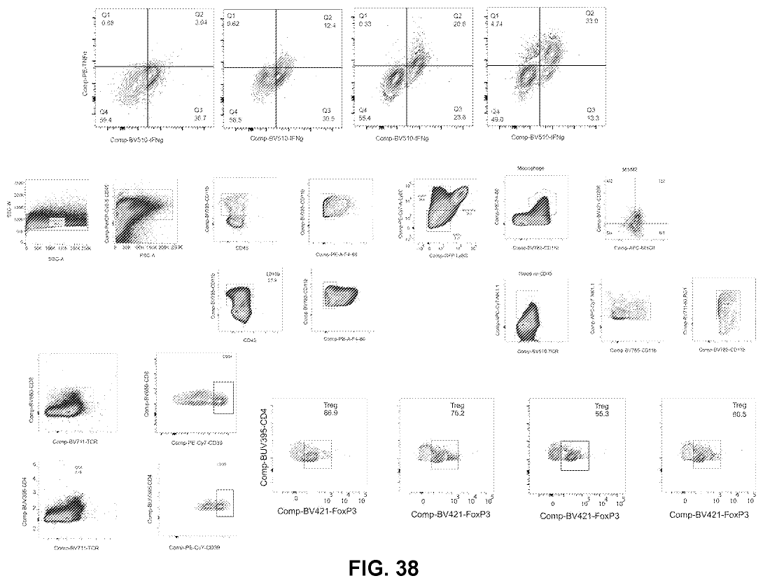

Tumor immune-environment responses were assessed in the s.c. grafted and orthotopic HCC models by cytokine detection and flow cytometry after treatment. RIL-175 tumor-bearing C57B1/6 mice (6-8 weeks old, n=3 per group) were systemically (i.v. via tail vein) injected with CTCE-targeted p53 mRNA NPs or control groups (i.e., PBS or CTCE-EGFP NPs) every 3 days for four injections (at the murine p53 or EGFP mRNA dose of 350 μg/kg animal body weight). For the combinatorial immunotherapy group, one day after each i.v. injection of CTCE-p53 NPs, mice underwent intraperitoneal (i.p.) administration of aPD1 (100 g per dose). The tumor inoculation and treatment schedule are depicted in FIG. 22a and FIG. 26a. Forty-eight hrs post treatment, mice were euthanized and tumor tissue was harvested and homogenized for flow cytometry and cytokine analysis. For flow cytometry, tumor tissues were resected and minced, and fragments were incubated in HBSS with 1.5 mg/mL of hyaluronidase and 15 μg/mL of collagenase for 30 minutes at 37° C. Digested tissues were passed through a 70-μm cell strainer and washed twice with phosphate-buffered saline (PBS)/0.5% bovine serum albumin. Prior to immunostaining, cells were washed with the buffer and fixed and permeabilized with FoxP3/Transcription Factor Staining Buffer Set (eBioscience/Thermo Fischer Scientific) to stain the intracellular markers. Harvested cells were incubated in Dulbecco's Modified Eagle Medium with cell activation cocktail with BD Leukocyte Activation Cocktail, with BD GolgiPlug™(1:500, Biolegend) for 6 h at 37° C. The cells were stained with the antibodies of cell surface and intracellular marker in the buffer with brefeldin A. Cells were stained with fluorescence-labeled antibodies CD11c (Biolegend, cat. no. 117310, clone N₄₁₈), CD80 (Biolegend, cat. no. 104722, clone 16-10A1), CD 86 (Biolegend, cat. no. 105005, clone GL-1), CD4 (Biolegend, cat. no. 100412, clone GK1.5), CD3 (Biolegend, cat. no. 100204, clone 17 A2), CD8 (Biolegend, cat. no. 140408, clone 53-5.8), CD11b (Biolegend, cat. no. 101208, clone M1/70), F4/80 (Biolegend, cat. no. 123116, clone BM8), CD206 (Biolegend, cat. no. 141716, clone C068C2), Gr-1 (Biolegend, cat. no. 108412, clone RB6-8C5), CD45 (Biolegend, cat. no. 103108, clone 30-F11), TCR (Biolegend, cat. no. 109243, clone H57-597), CD39 (Biolegend, cat. no. 143805, clone Duha59), Ki67 (Biolegend, cat. no. 652423, clone 16A8), CD11b (Biolegend, cat. no. 101243, clone M1/70), CD206 (Biolegend, cat. no. 141717, clone C068C₂), Forkhead box protein P3 (FoxP3; Biolegend, cat. no. 126419, clone MF-14), IFN-7 Receptor βchain (Biolegend, cat. no. 113605, clone MOB-47), CD 119 (BD Bioscience, cat. no. 740897, clone GR20), FITC (Biolegend, cat. no. 503805, clone JES6-5H4) following the manufacturer's instructions. All antibodies were diluted 200 times, except for FoxP3 and CD119 staining, which were 1:100 dilution. The stained cells were measured on a flow cytometer (Accuri C6 Plus, BD Biosciences) and analyzed by FlowJo software (Flowjo V10). The numbers presented in the flow cytometry analysis images are percentage based. A non-limiting flow cytometry gating strategy is provided in FIG. 38. For cytokine studies, tissue samples were assayed in duplicate using the MSD proinflammatory Panel I, a highly sensitive multiplex enzyme-linked immunosorbent assay (ELISA) for quantitatively measuring 10 cytokines-IFN-γ, interleukin (IL)-1β, IL-2, IL-4, IL-5, IL-6, IL-10, IL-12p70, TNF-α, KC/GRO and IL-9, IL-15, IP-10, MCP-1, MIP-1la, MIP-2, IL-17A/F, IL-27p28/IL-30, and IL-33 using electrochemiluminescence-based detection (MesoScale Discovery, Gaithersburg, MD).

### In Vivo Therapeutic Efficacy

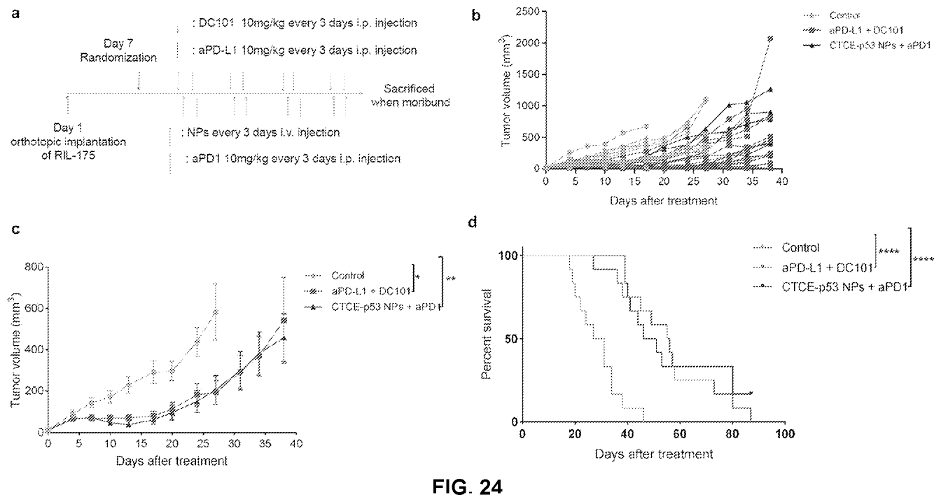

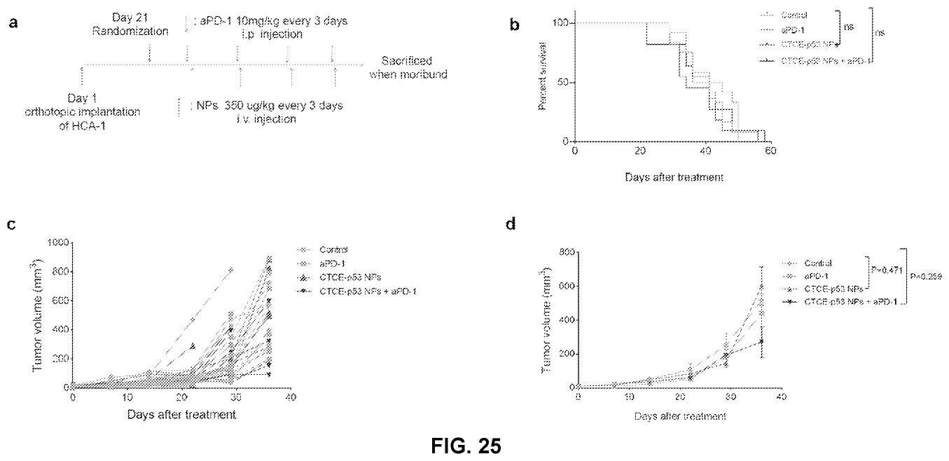

The therapeutic effects of p53-mRNA NPs and their integrated antitumor effect with anti-PD1 were evaluated in the p53-null HCC s.c. RIL-175 tumor model, p53-null RIL-175 orthotopic tumor model, and p53-wild-type HCA-1 orthotopic tumor model. For the s.c. model, RIL-175 tumor-bearing C57B1/6 mice (6-8 weeks old, n=5 per group) were monitored for tumor growth every other day after tumor implantation; tumor size was measured using a digital caliper and calculated as 0.5×length×width². When the tumor volume reached about ˜100 mm³, mice were randomly divided into five groups (n=5), which received treatment with PBS, CTCE-EGFP NPs, CTCE-p53 NPs, aPD1, or the combination of CTCE-p53 NPs and aPD1 according to the schedule in FIG. 26a at the mRNA dose of 350 μg/kg animal body weight, while the aPD1 were administrated by i.p. at 100 g per dose one day after the p53-mRNA NPs treatment. Tumor growth was measured and calculated every 3 days. The body weights of all mice were recorded every three days during this period. Animals were euthanized upon showing signs of imperfect health or when the size of their accumulated tumors exceeded 1.0 cm³. For the orthotopic HCC tumor model, tumor growth was monitored by high-frequency ultrasonography every 3 days. When the tumor size reached ˜5 mm in diameter, mice were randomly assigned to a treatment group (n=12). Treatments were administered according to the schedule in FIG. 22a. For the comparison of side-by-side the in vivo survival of the combination of CTCE-p53 NPs with aPD1 against the new standard of care in HCC patients (i.e., anti-VEGFR2 antibody+aPD-L1 antibody) in the orthotopic RIL-175 tumor model, treatments were administered i.p. every 3 days for 4 doses at 10 mg/kg of aPD-L1 antibody (Bioxcell, #BE0101, clone 10F.9G2), and 10 mg/kg of anti-VEGFR-2 antibody (Bioxcell, #BE0060, clone DC101) (FIG. 24a). For survival studies, the endpoint was moribund status, defined as signs of prolonged distress, >15% weight loss compared with the starting date, body condition score>2, or tumor size of >15 mm in diameter.

### Bioluminescence

To further explore the therapeutic efficacy of our therapeutic strategy, tumors were also assessed using an in vivo bioluminescence imaging system (Bruker Xtreme scanner). Mice were monitored for tumor growth by bioluminescent in vivo imaging every 6 days (Day 0, 6, and 12); specifically, 8 minutes after intraperitoneal injection of 150 mg/kg D-luciferin substrate (PerkinElmer, Catalog #122799), mice from each treatment group (n=3) were imaged.

### Immunohistochemistry Staining

The expression of p53 protein and CD8+cells in tumor tissue sections from different in vivo treatment groups were assessed by immunohistochemistry. Tumor sections were fixed in 4% buffered formaldehyde solution and embedded in paraffin. Paraffin-embedded sections were deparaffinized, rehydrated, and washed in distilled water. In order to retrieve the antigen, tumor tissue sections were incubated in 10 mM citrate buffer (pH=6) for 30 min, washed in PBS, and immersed in 0.3% hydrogen peroxide (H₂O₂) for 20 min, then incubated in blocking buffer (5% normal goat serum and 1% BSA) for 60 min. Tissue sections were then incubated with the appropriate primary antibodies (PBS solution supplemented with 0.3% Triton X-100) at 4° C. overnight in a humid chamber. After being rinsed with PBS, the samples were incubated with biotinylated secondary antibody at room temperature for 30 min, rinsed again with PBS, and incubated with the avidin-biotin-horseradish peroxidase complex (ABC kit, Vector Laboratories, Inc.). After being washed again, stains were processed with the diaminobenzidine peroxidase substrate kit (Impact DAB, Vector Laboratories, Inc.) for 3 min. Sections were evaluated using a Leica Microsystem after being counterstained with hematoxylin (Sigma), dehydrated, and mounted.

### In Vivo Toxicity Evaluation

The in vivo toxicity of p53-mRNA NPs was comprehensively studied in both the p53-null HCC s.c. graft tumor model and the p53-null orthotopic HCC tumor model. In brief, the major organs were harvested at the end point, sectioned, and H&E stained to evaluate the histological differences. In addition, blood was drawn, and serum was isolated at the end of the in vivo efficacy experiment. Various parameters including ALT, AST, BUN, RBC, WBC, Hb, MCHC, MCH, HCT, and LY were tested to evaluate toxicity.

### Statistical Analysis

A two-tailed Student's t-test or a one-way analysis of variance (ANOVA) was performed when comparing two groups or more than two groups, respectively. Statistical analysis was carried out using Prism 8.0 (GraphPad) and Microsoft Excel. Data are expressed as standard deviation (S.D.) or standard error means (S.E.M.) as described herein. Difference was considered to be significant if P<0.05 (*P<0.05, **P<0.01, ***P<0.001, ****P<0.0001 unless otherwise indicated). All studies were performed at least in triplicate, unless otherwise stated.

### Example 3: Engineering and Optimization of CXCR4-Targeted mRNA NPs

We developed a robust self-assembly strategy for formulating polymer-lipid hybrid NPs for mRNA delivery (see, e.g., ref. 27-28) composed of the ionizable lipid-like compound G₀-C₁₄ for mRNA complexation, a biocompatible poly(lactic-co-glycolic acid) (PLGA) polymer for forming a stable NP core to carry the GO-C₁₄/mRNA complexes, and a lipid-poly(ethylene glycol) (lipid-PEG) layer for stability. We here engineered the hybrid NPs (FIG. 1a) for selective HCC targeting and high mRNA transfection efficiency.

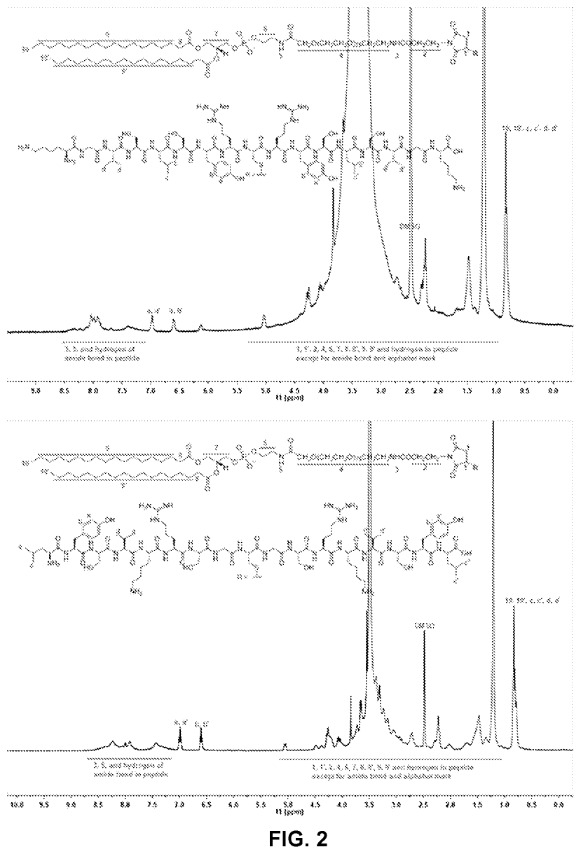

To improve HCC targeting, we modified the NPs with the targeting peptide CTCE-9908 (KGVSLSYRCRYSLSVGK; referred to as CTCE, SEQ ID NO: 1), which is specific to CXCR4, a chemokine receptor that is upregulated in cancer cells and is a validated selective target in HCC.²⁹⁻³⁰ for comparison, we also prepared non-targeted NPs using a scrambled peptide (LYSVKRSGCGSRKVSYL; referred to as SCP, SEQ ID NO: 2). The CTCE or SCP peptide was first conjugated to 1,2-distearoyl-sn-glycero-3-phosphoethanolamine-N-[maleimide(polyethylene glycol)-3000](DSPE-PEG-Mal) by the thiol-maleimide Michael addition click reaction, with a high chemical yield (≥82%). The chemical structures of DSPE-PEG-CTCE and DSPE-PEG-SCP were confirmed by ¹H-NMR analysis (FIG. 2).

To optimize the targeting efficacy of the mRNA NPs, we examined the effect of CTCE peptide surface density on the cellular uptake of RIL-175 murine HCC cells. As shown in FIG. 1b, CTCE-conjugated enhanced green fluorescent protein (EGFP) mRNA NPs (referred to herein as CTCE-EGFP NPs) showed significantly greater cellular uptake compared to non-targeting SCP EGFP mRNA NPs (referred to as SCP-EGFP NPs) due to the active targeting ability of the CTCE peptide towards HCC cells. We found that 5% or 6% CTCE peptide provided maximum cellular uptake in RIL-175 cells while maintaining NP stability. The uptake of the 5% CTCE-EGFP NPs was >15-fold higher than that of the 5% SCP-EGFP NPs, which was also confirmed by confocal fluorescence microscopy in RIL-175 cells (FIG. 1c). The 5% peptide density was selected for further analyses.

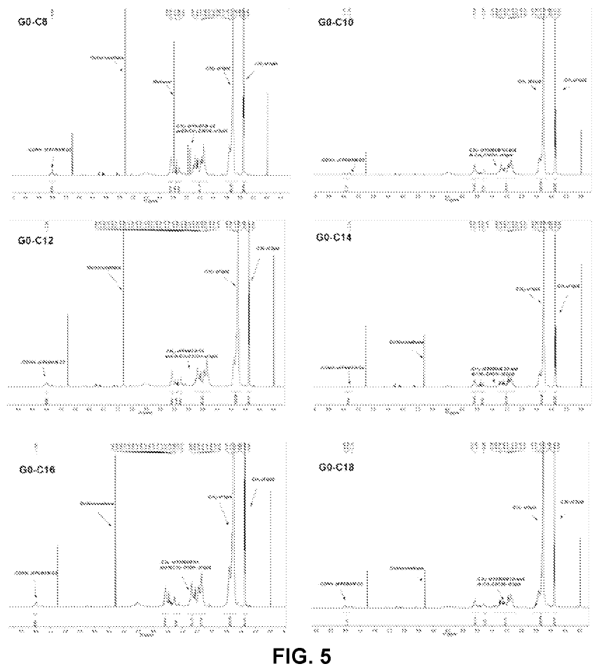

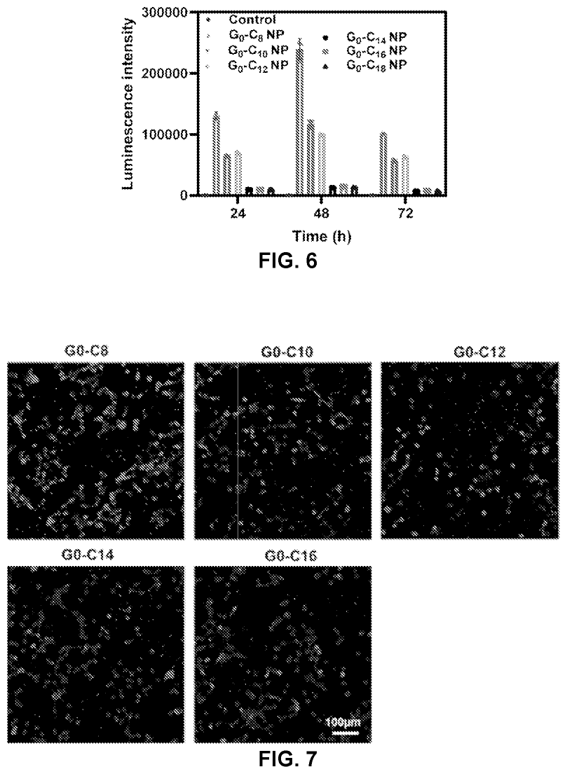

To identify efficacious ionizable lipid-like materials for mRNA complexation and translation, a series of G₀-Cₙ compounds (FIG. 3a) was synthesized through ring opening of epoxides by generation 0 of poly(amidoamine) (PAMAM) dendrimers (FIG. 3b) and screened for using a model luciferase-mRNA. The chemical structures of G₀-Cₙ were confirmed by ¹H-NMR spectrum (FIG. 5). Analysis of luciferase-mRNA NPs transfection results (FIG. 1d and FIG. 6) showed that G₀-C₈ had effective mRNA transfection ability, and this compound was chosen as the ionizable lipid-like material for formulating targeted mRNA NPs for in vivo treatments. To explore the possible mechanisms behind this, we studied the mRNA encapsulation efficiency and cellular uptake of the mRNA NPs formulated with different G₀-Cₙ. As shown in the Table 2, G₀-Cₙ had negligible effect on the mRNA encapsulation efficacy. However, their effect on cellular uptake seemed to play an important role for the mRNA delivery efficacy (FIG. 7), with the G₀-C₈ NP showing higher cellular uptake than other G₀-Cₙ NPs in this non-limiting instance.

TABLE 2
Effect of different cationic lipid-like materials G₀-Cₘ
on the encapsulation efficacy of Cy5-Luciferase mRNA NPs

| | G₀-C₈ | G₀-C₁₀ | G₀-C₁₂ | G₀-C₁₄ | G₀-C₁₆ |
|---|---|---|---|---|---|
| Encapsulation efficiency | 67.3 | 65.9 | 63.7 | 66.9 | 58.3 |
| (EE) % | | | | | |

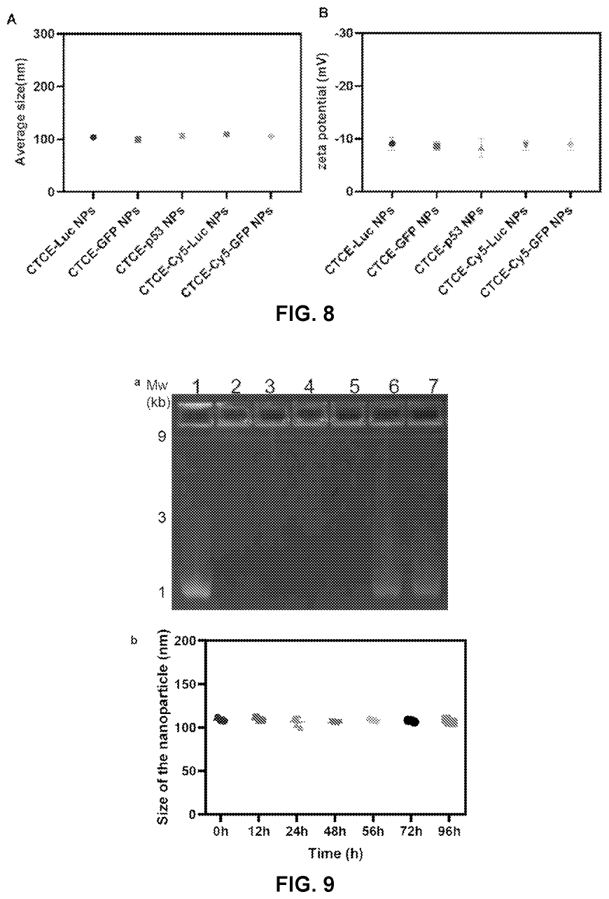

The hybrid CTCE-conjugated p53 mRNA NPs (referred heretofore as CTCE-p53 NPs) were ˜110 nm in size as measured by dynamic light scattering (DLS), and their spherical and uniform structure was confirmed by transmission electron microscopy (TEM) imaging (FIG. 1e,f). The addition of the targeting ligand (CTCE) and the scrambled peptide (SCP) to the NP surface slightly increased the particle size as well as the zeta potential, due to the positive charges of both peptides (FIG. 1f). In addition, we characterized all the nanoformulations used in this study, including Luc mRNA NPs, GFP mRNA NPs, and p53 mRNA NPs. As shown in FIG. 8, all the nanoformulations used in this study exhibited similar average size and zeta potential.

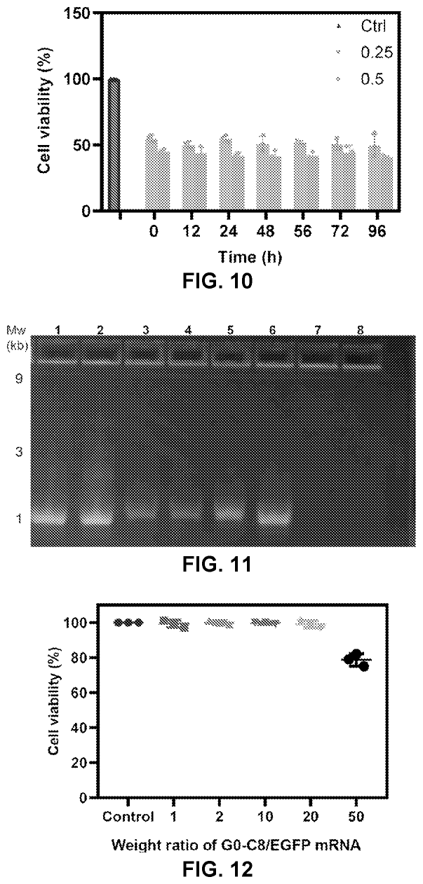

The organic solvent DMF (dimethylformamide) had no effect on the integrity or stability of EGFP mRNA, either as naked mRNA or encapsulated in NPs (FIG. 9a). Moreover, we detected no obvious changes in the size of p53-mRNA NPs over a period of 96 h in the presence of 10% serum, suggesting the in vivo stability of our targeted mRNA NPs (FIG. 9b). To further evaluate the stability of the p53-mRNA NPs, the cell viability was measured using RIL-175 cells after treatment with p53-mRNA NPs pre-incubated with 10% serum for various time points up to 96 h (at 37° C.). Comparable cell viability in all the groups (FIG. 10) further supported the stability of these p53-mRNA NPs.

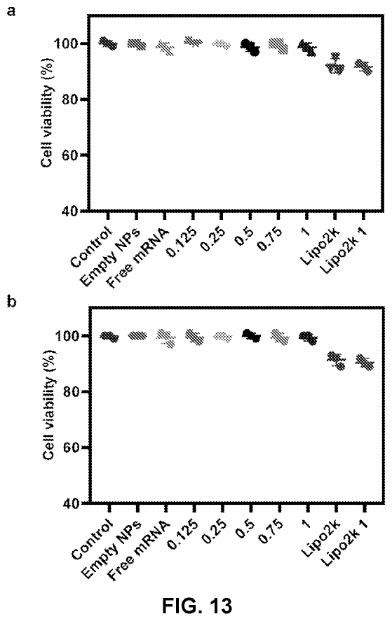

In particular, pH played a role in complexing mRNA for the ionizable G₀-C₈, and effective mRNA complexation with G₀-C₈ was achieved in acidic conditions. As shown by agarose gel electrophoresis assay at pH 7.4 (FIG. 11), G₀-C₈ could not fully complex mRNA even at a weight ratio of 200 G₀-C₈/mRNA. In comparison, when the pH was adjusted to 3.5 in citrate buffer solution, the mRNA could be completely complexed at a weight ratio of G₀-C₈/mRNA as low as 2. In addition, this ratio is favorable for mRNA delivery in vivo because it reduces the need to use ionizable lipid-like materials and may, in some non-limiting instances, improve the safety of the mRNA NPs. A cytotoxicity assay was further performed to evaluate the in vitro cytotoxicity of G₀-C₈/EGFP mRNA (FIG. 12), which showed ˜100% viability at various ratios of G₀-C₈/mRNA from 1 to 20 in RIL-175 cells. In addition, in vitro cytotoxicity was further examined in both RIL-175 and normal hepatocyte THLE-3 cells. The near-100% cell viability at all tested concentrations in both cell lines (FIG. 13) indicated the safety of these mRNA NPs.

### Example 4: CXCR4-Targeting Improves mRNA NP Delivery to HCC Cells In Vitro and In Vivo

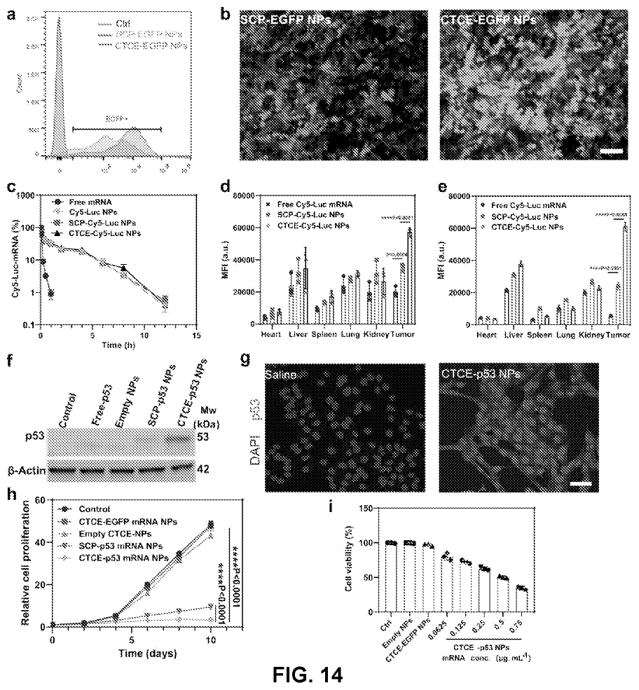

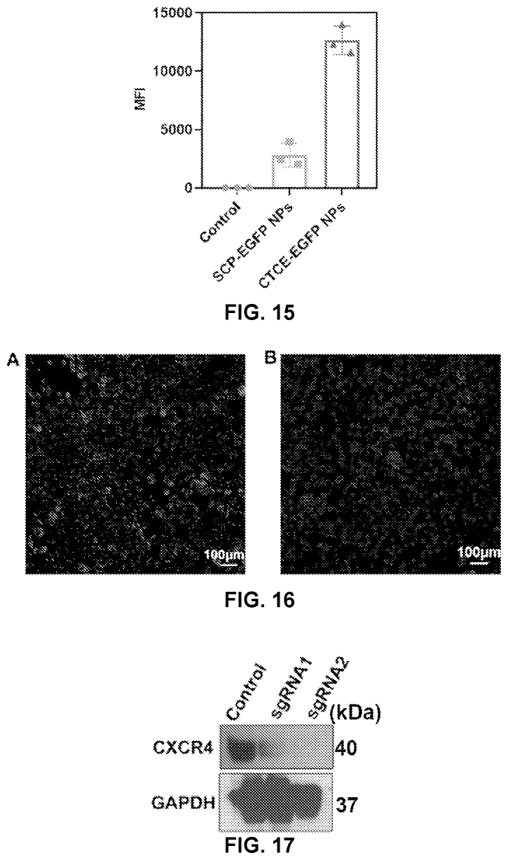

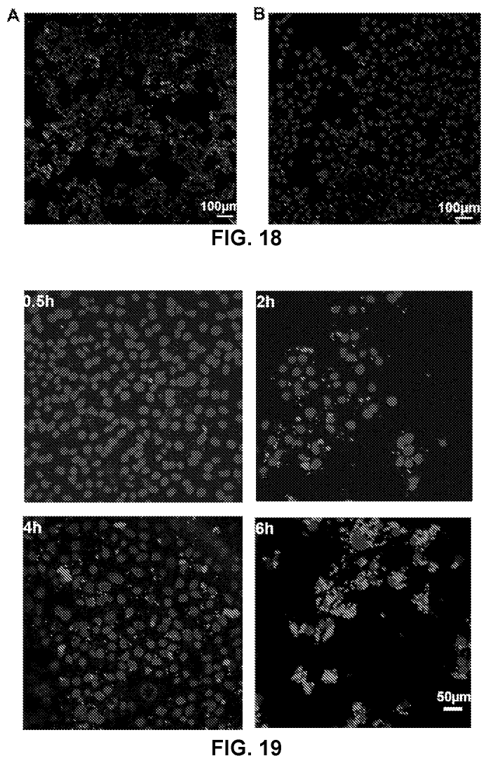

We then investigated the CTCE-targeting effect of our nRNA NPs on cellular uptake and mJRNA transfection inp33-deficient murine I-CC cells (R-L,-175) using flow cytometry. We first examined the transfection efficacy of the targeted mRNA NPs and non-targeted mRNA NPs in vitro using EGFP-mRNA as the model mRNA, by counting EGFP-positive cells (FIG. 14a). Both SCP-EGFP NPs and CTCE-EGFP NPs showed higher fractions (>90%) of EGFP-positive cells after mRNA NP-transfection compared to controls (free/naked EGFP mRNA). Notably, the CTCE-EGFP NPs induced a ˜4.5-fold higher mean fluorescence intensity in cells, as compared to the SCP-EGFP NPs (FIG. 15). The higher transfection efficiency of CTCE-EGFP NPs was confirmed by fluorescence microscopy (FIG. 14b). To further verify the selectivity of the CTCE-mRNA NPs, we also examined the targeting effect of CTCE peptide by blocking the CXCR4 receptor on RIL1-75 cell surface using free CTCE peptide (FIG. 16). Upon treatment with CTCE peptide, the fluorescence intensity of RIL-175 cells co-incubated with CTCE-Cy5-Luciferase mRNA NPs was significantly lower than that without blocking. Moreover, we generated a CXCR4-knockout (CXCR4-KO) RIL-175 cell line by CRISPR/Cas9 editing and performed in vitro cellular uptake. As evidenced by Western blotting (WB, FIG. 17) CXCR4 expression of the RIL-175 cells were effectively knocked out by CRISPR/Cas9 editing. In vitro cellular uptake study (FIG. 18) showed that the fluorescence intensity of CXCR4-KO RIL-175 cells co-incubated with CTCE-Cy5-Luciferase mRNA NPs was significantly reduced than that of the sgControl RI.-175 cells (without CXCR4-KO). These results demonstrate the CXCR4-mediated active targeting effect of the CTCE-NPs on the RIL-175 cell line.

Next, intracellular uptake of the mRNA NPs in RIL-175 cells was examined by confocal fluorescence microscopy after incubating Cy5-labeled Luciferase-mRNA NPs (CTCE-Cy5-Luc NPs) with RIL-175 cells for 0.5, 2, 4, or 6 hrs. The intensity of red fluorescence from Cy5-Luc mRNA in the cells increased in proportion to incubation time (FIG. 19), suggesting the successful intracellular delivery of our mRNA NPs.

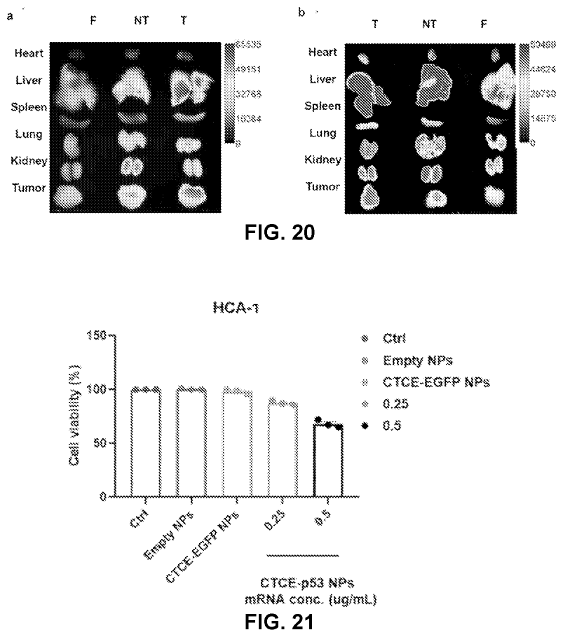

To test the efficiency of CXCR4-mediated HCC-targeting of CTCE-mRNA NP delivery in vivo, we next conducted pharmacokinetics (PK) and biodistribution (BioD) studies. We first evaluated PK parameters by administering targeted or non-targeted Cy5-Luc-mRNA NPs or free Cy5-Luc-mRNA into healthy C57B1/6 mice via the tail vein. The PK results showed that free mRNA was rapidly cleared, with a dramatic decrease to ˜8% after 15 min (FIG. 14c). In contrast, similar to Cy5-Luc NPs without peptide modification, both SCP-Cy5-Luc NPs and CTCE-Cy5-Luc NPs showed prolonged mRNA circulation, with >30% of the Cy5-Luc-mRNA still circulating after 60 min. After 4 h, nearly 20% of both NPs were still detectable, while most free mRNA was cleared within 1 h. This result also indicated that the presence of the targeting moiety (i.e., CTCE) did not alter the PK profile of the mRNA NPs. We then evaluated the BioD and tumor accumulation of these NPs in both orthotopically and ectopically (s.c.) grafted RIL-175 HCCs. Tumor-bearing mice were administered free Cy5-Luc-mRNA, non-targeted SCP-Cy5-Luc-mRNA NPs, or targeted CTCE-Cy5-mRNA NPs by tail vein. As shown in FIG. 14d,e and FIG. 20, in both HCC models, both NPs exhibited considerable intratumoral accumulation, while the fluorescent signal of free Cy5-mRNA was barely detectable in the tumor tissue 24 h post-injection. Notably, there was ˜1.5 and 2.7-fold greater intratumoral accumulation of CTCE-targeted NPs than non-targeted NPs in the orthotopic and ectopic models, respectively. Taken together, the evidence suggests that CTCE-targeted NPs demonstrated significantly enhanced cellular uptake, mRNA transfection efficiency, and intratumoral accumulation compared to non-targeted NPs irrespective of tumor site/stroma, supporting the use of CTCE peptide ligands for selective HCC cell targeting. Such testing could be implemented to test the effectiveness of other targeting ligands in selectively targeting other cell types.

### Example 5: Cxcr4-Targeted mRNA Np Increases P53 Protein Expression and Reduces HCC Cell Viability In Vitro

To determine whether the targeted p53-mRNA NPs could induce the expression of therapeutic p53 inp53-null RIL-175 cells, we first checked p53 protein expression after treatment with CTCE-p53 NPs versus SCP-p53 NPs. Both WB and immunofluorescence (IF) staining (FIG. 14f,g) confirmed the successful restoration of p53 expression in RIL-175 cells. The WB data further showed that targeted NPs exhibited enhanced level of p53 expression compared with non-targeted NPs. In addition, the IF images showed that p53 protein was mainly localized in the cytoplasm of RIL-175 cells. Next, we tested cell growth and cell viability after treatment with CTCE-p53 NPs versus SCP p53 NPs. FIG. 14h shows that the number of viable cells was dramatically decreased after 10-day treatment with SCP-p53 NPs or CTCE-p53 NPs compared to control-treated cells, or to cells treated with CTCE-EGFP NPs or empty CTCE-NPs. Of note, the CTCE-p53 NPs elicited greater growth inhibition than non-targeted SCP-p53 NPs, consistent with higher p53 expression. Moreover, CTCE-p53 NPs significantly decreased cell viability in a dose-dependent manner compared to the control, free mRNA, and control NPs (FIG. 14i). These results indicate that the CTCE-targeting NP system effectively delivers p53 mRNA to HCC cells, restoring functional p53 activity and reducing HCC cell viability.

In addition, we tested whether the CTCE-p53 NPs could induce the suppressing function of p53 inp53-wild type murine HCC cell line HCA-1. As shown in FIG. 21, modest cytotoxicity was observed at high doses in HCA-1 cells, whereas empty NPs and control NPs (CTCE-EGFP NPs) had no effects on HCA-1 cell viability.

### Example 6: Combining CXCR4-Targeted p53 mRNA NPs with PD-1 Blockade Inhibits Tumor Growth and Reprograms the Immune TME in Orthotopic p53-Null Murine HCC

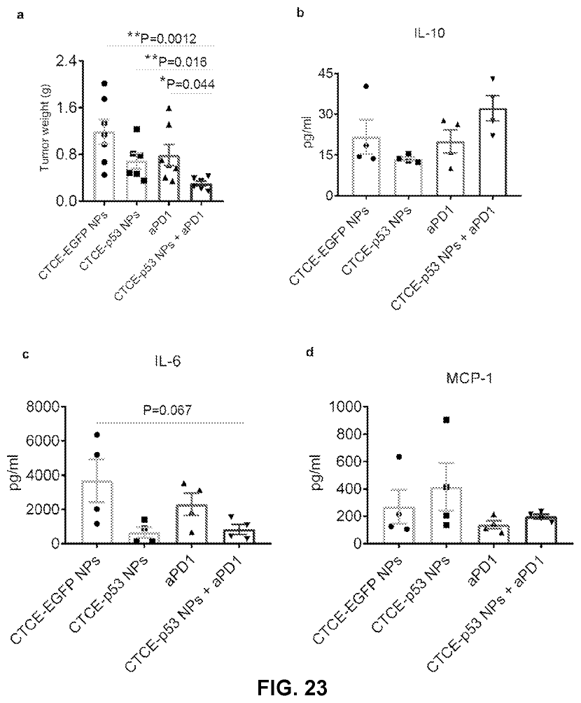

To examine the role of p53 in immunosuppression in HCC, we tested the CTCE-p53 NPs and aPD1 againstp53-null HCC. Mice with established orthotopic RIL-175 tumors were treated with either CTCE-p53 NPs at a mRNA dose of 350 μg/kg by intravenous (i.v.) injection, aPD1 by intraperitoneal (i.p.) injection, or their combination, every 3 days for 4 cycles (FIG. 22a). Tumor growth was monitored by high-frequency ultrasound imaging (FIG. 22b). In vivo results revealed that CTCE-p53 NPs treatment or aPD1 therapy alone inhibited HCC growth compared to IgG-treated control mice, but their combination was significantly more effective than either treatment alone (individual growth curves in FIG. 22c, mean tumor volumes in FIG. 22d, and mean tumor weight in FIG. 23a). We also performed immunohistochemistry (IHC) analysis to confirm the expression of p53 in the orthotopic tumors. As shown in FIG. 22e, p53 was expressed at the highest levels in the CXCR4-p53 NP-treated groups, confirming the successful delivery of p53 mRNA to the orthotopic tumors.

We then examined the impact of treatment on immune cell infiltration and activation in the RIL-175 tumors by flow cytometry analyses of digested HCC tissues. Compared to treatment with CTCE-EGFP NPs, CTCE-p53 NPs, or aPD1 alone, we found that the combination of CTCE-p53 NPs with aPD1 significantly increased the number of infiltrating CD8⁺ T cells (FIG. 22f). Importantly, the fraction of activated (IFN-γ*TNF-α⁺) CD8⁺ T cells was significantly increased in the HCC tissue after combination therapy (FIG. 22g). In addition, the fraction of infiltrating CD4*FoxP3-effector T cells (FIG. 22h), mature (KLRG1⁻CD11b⁺) NK cells (FIG. 22i,j), and activated (IFN-7⁻ and IFN-7R⁻) NK cells (FIG. 22k,l) all increased after combined treatment with CTCE-p53 NPs and aPD1.

Moreover, we found that combination therapy effectively polarized tumor-associated macrophages (TAMs) towards the M1-like phenotype and decreased M2-like TAMs in HCC (FIG. 22m,n). It is worth noting that CTCE-p53 NPs alone increased the fractions of mature NK cells and M1 TAMs while reducing M2 TAMs (FIG. 22l-n); in contrast, aPD1 alone had the opposite effect by polarizing TAMs toward the M2-phenotype (FIG. 22m,n). We also examined changes in key immune cytokines post-treatment by multiplexed array analysis of whole tumor tissue protein extract. We found that CTCE-p53 NPs and aPD1 significantly increased TNF-α and IL-10 levels; they also tended to increase IFN-γ⁺ and IL-2 and decrease IL-6 but neither IL-10 nor MCP1 (CCL2) (FIG. 22o-q and FIG. 23b-d). Collectively, these results suggest that the combination of CTCE-p53 NPs and PD-1 blockade effectively and globally reprogrammed the immune TME of HCC by increasing effector immune cells and cytokine levels in the tumor.

We further compared side-by-side the survival benefit of the combination of CTCE-p53 NPs with aPD1 against a regimen similar to the new standard of care in HCC patients (i.e., anti-VEGFR2 antibody (DC101)+aPD-L1 antibody) in the orthotopic RIL-175 tumor model (FIG. 24). Results showed that both treatments were effective and comparable in increasing overall survival and delaying disease morbidity in the p53-null murine HCC model. In addition, the in vivo therapeutic efficacy of the combination of CTCE-p53 NPs with aPD1 were also evaluated in an orthotopic p53-wild type HCC tumor model (HCA-1) in C₃H mice. Though the CTCE-p53 NPs showed modest in vitro cytotoxicity in HCA-1 cells (FIG. 21), this modest in vitro effect did not translate into an in vivo survival benefit (FIG. 18) with the same dosage and dosing frequency used in the RIL-175 model.

### Example 7: Combining CXCR4-Targeted p53 mRNA NPs with PD-1 Blockade is Effective in Ectopic p53-Null Murine HCC

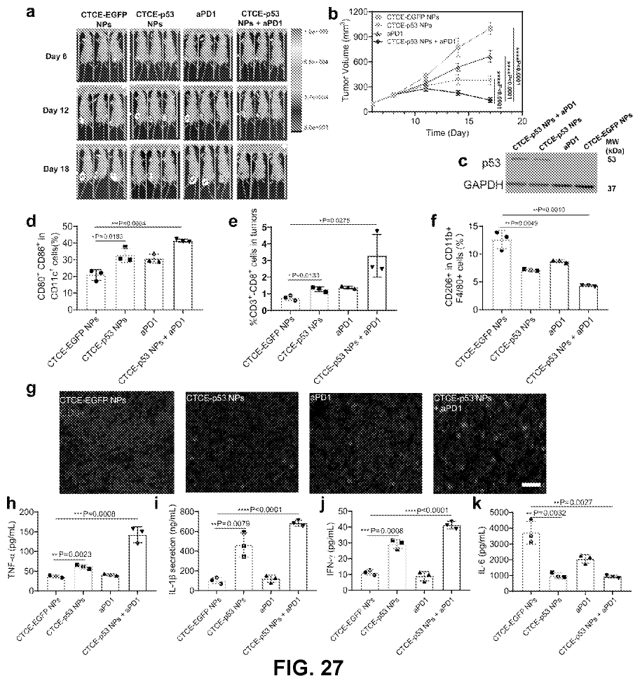

To determine whether the comprehensive reprogramming of the immune TME was dependent on the localization of tumor within the liver, we next evaluated in vivo p53 expression, anti-tumor immune response, and anti-tumor efficacy in a subcutaneously grafted HCC model in immunocompetent C57B1/6 mice. We administered four injections of CTCE-p53 NPs i.v. (350 μg/kg body weight) and aPD1 i.p. (100 g per dose) every 3 days in mice with established tumors (FIG. 26a). Tumor-bearing mice treated with CTCE-EGFP NPs served as controls. We first evaluated the anti-tumor effect of CTCE-p53 NPs and aPD1 by bioluminescence imaging of the luciferase-expressing RIL-175 tumors to estimate viable tumor burdens (FIG. 27a). The combination treatment markedly limited the increase of bioluminescence signals compared to CTCE-p53 NPs or aPD1 treatment alone, indicating a potent anti-tumor effect. Moreover, RIL-175 tumor-bearing mice treated with CTCE-EGFP NPs showed aggressive tumor growth, while aPD1 treatment and CTCE-p53 NPs alone delayed the growth of RIL-175 tumors (FIG. 26b and FIG. 27b). The combination of CTCE-p53 NPs with anti-PD1 showed a significantly greater anti-tumor effect than either treatment alone, significantly reducing tumor volume and inducing tumor regression after 4 cycles of treatment (FIG. 27b).

Next, protein extracts from tumor tissues from the different treatment groups were analyzed by WB. As shown in FIG. 27c, CTCE-p53 NP treatment alone and combined with aPD1 treatment both elicited high levels of p53 protein expression in ectopic p53-null RIL-175 tumors, whereas neither the aPD1 nor the control NPs (i.e., CTCE-EGFP NPs) had any effect on p53 expression. IHC analysis of tumor sections further confirmed p53 expression (FIG. 26c). These results demonstrate that the p53 mRNA NPs effectively restored p53 expression in vivo and significantly enhanced the anti-tumor effects of aPD1 therapy in HCC growing outside the liver.

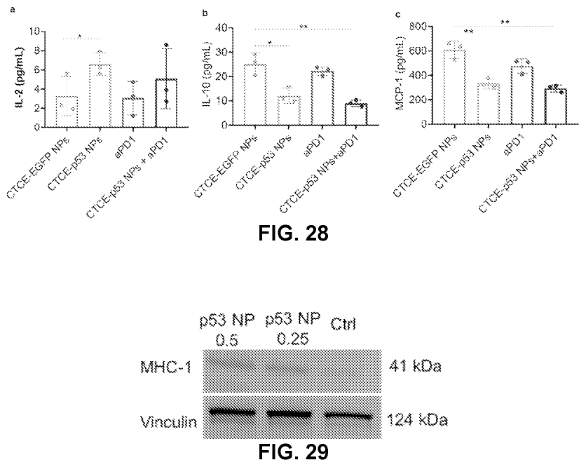

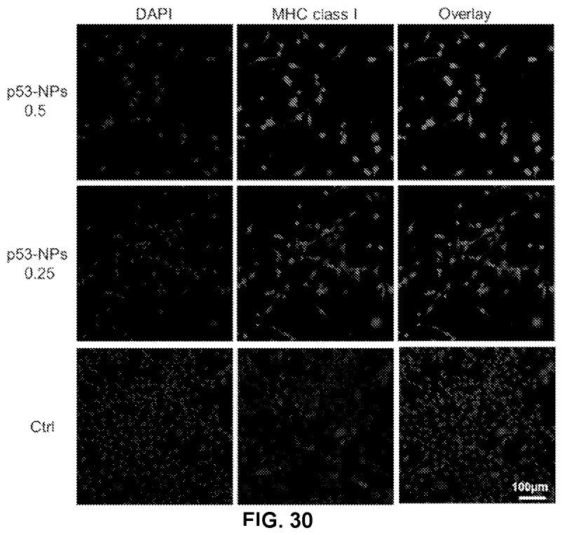

Using the same model, we also harvested tumors and lymph nodes to examine the number and phenotype of immune cells and the changes in secreted cytokines after four cycles of treatment. CTCE-p53 NPs alone or in combination with aPD1 induced a significant increase in CD80⁺CD86⁻ lymph node-resident dendritic cells (LNDCs) and intratumoral CD8⁺ T cells (FIG. 27d,e), and a significant decrease in M2-type TAMs (FIG. 27f). IF analysis of tumor tissues confirmed the increased intratumoral infiltration by CD8⁺ T cells after combination treatment (FIG. 27g). Multiplexed array analysis revealed, similar to orthotopic HCCs, increased expression of cytokines associated with immune cell activation (e.g., TNF-α, IL-1β, IFN-γ, and IL-2) and also decreased expression of immunosuppressive cytokines (e.g., IL-10 and MCP-1) in the ectopic HCCs after combination treatment (FIG. 27h-k and FIG. 28). Moreover, we also studied the role of p53 on MHC class I expression by WB and IF. Results in FIGS. 29 and 30 revealed an association between p53 and MHC class I expression, indicating the potential role of p53 restoration in inducing immune responses. These results demonstrate that targeting HCC cells with CTCE-p53 NPs combined with aPD1 therapy triggers anti-tumor immunity and reprograms the immune TME of HCC both in the liver and in other organs.

### Example 8: Combination Therapy Prolongs Survival and Reduces Bloody Ascites, Pleural Effusions, and Lung Metastases

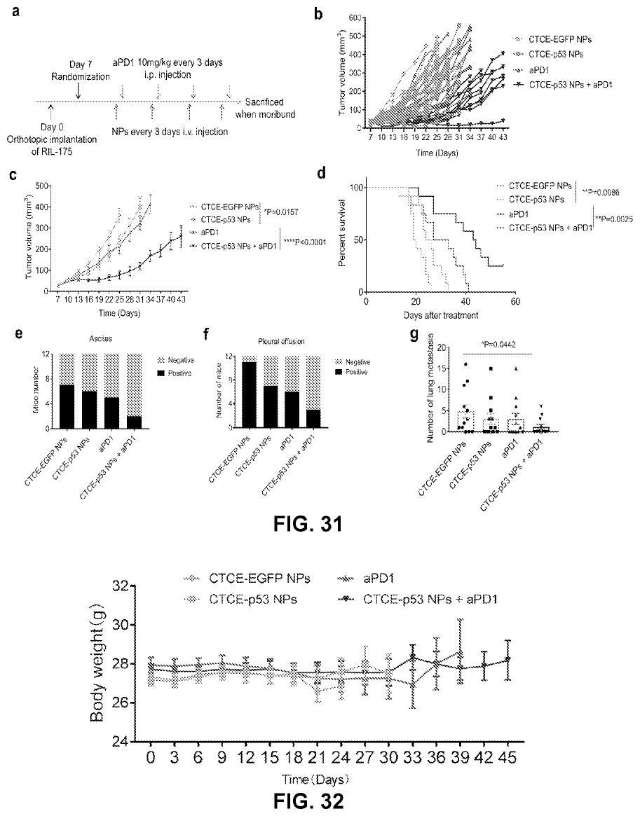

Using the orthotopic RIL-175 tumor model, we further evaluated the therapeutic efficacy of combining aPD1 with CTCE-p53 NPs in mice with established tumors (FIG. 31a). We treated the mice by i.v. injection of NPs and i.p. injection of aPD1 for four cycles and then monitored the tumor growth by ultrasound imaging and the survival. CTCE-p53 NPs alone and aPD1 alone modestly inhibited tumor growth, but their combination elicited a significant delay in tumor growth (FIG. 31b,c). Notably, the group treated with CTCE-p53 NPs plus aPD1 showed a significant and substantial survival benefit (median overall survival of 43.5 days, almost double that in the control group in this model, HR=0.26; p=0.0001) (FIG. 31d). In addition, only the combination treatment reduced the incidence of bloody ascites (FIG. 31e) and pleural effusions (FIG. 31f), which are potentially lethal adverse effects of orthotopic HCC. Moreover, when we assessed the lung metastatic burden by enumerating metastatic nodules, we found it significantly reduced in the group that received a combination of CTCE-p53 NPs with aPD1 (FIG. 31g). These findings suggest that p53 restoration using CXCR4-targeted mRNA NPs can markedly improve the efficacy of aPD1 therapy in p53-deficient HCC.

### Example 9: Combination of p53 mRNA NPs with aPD1 is Safe In Vivo

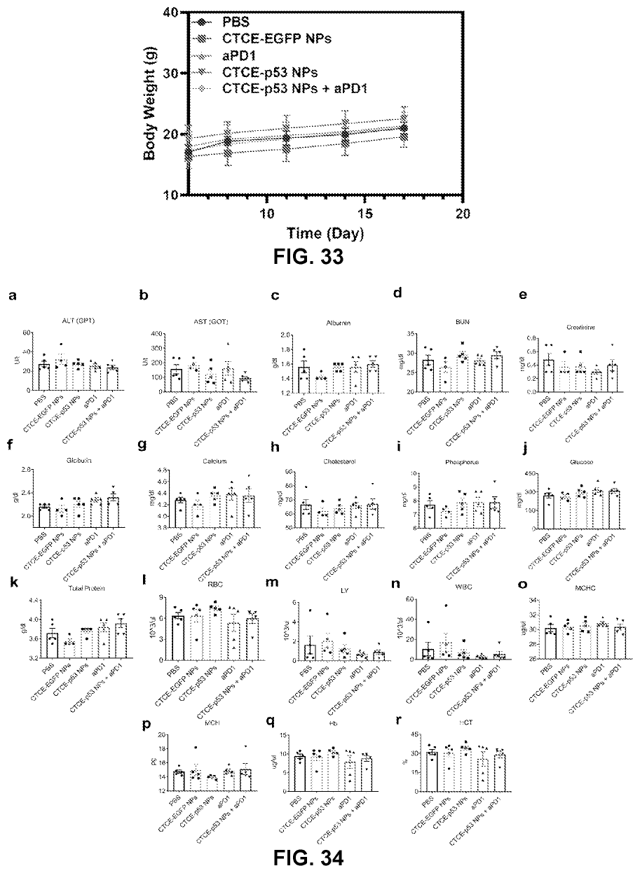

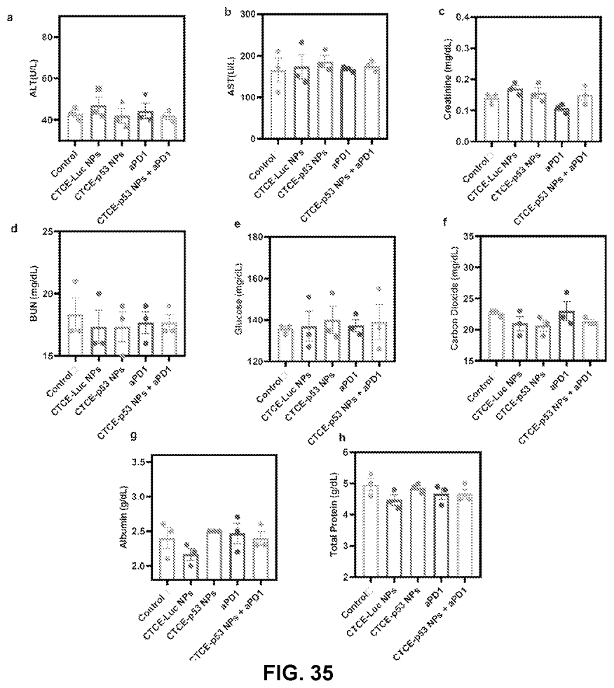

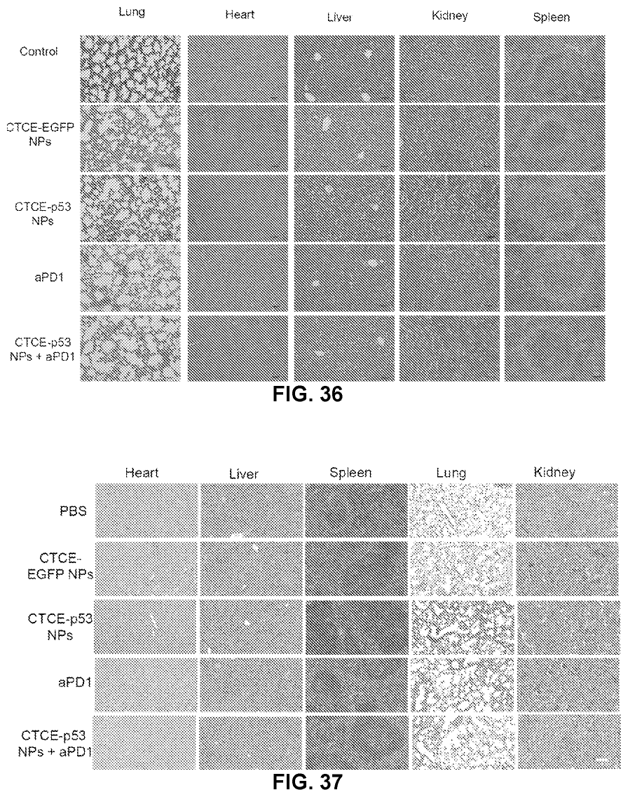

Finally, to evaluate the in vivo safety of CXCR4-targeted p53-mRNA NPs alone and in combination with aPD1, mouse weight was monitored during the above animal studies with the s.c. grafted and orthotopic models, and blood and major organs (e.g., heart, kidneys, liver, lung, and spleen) were harvested at the end of these studies. No significant change in body weight was observed in any of the treatment groups (FIGS. 32 and 33). We performed hematological analysis based on serum biochemistry and whole blood panel tests. A series of parameters were tested, including alanine aminotransferase (ALT), aspartate aminotransferase (AST), urea nitrogen (BUN), albumin, BUN, creatinine, globulin, calcium, cholesterol, phosphorus, glucose, total protein, red blood cells (RBC), white blood cells (WBC), hemoglobin (Hb), mean corpuscular hemoglobin concentration (MCHC), mean corpuscular hemoglobin (MCH), hematocrit (HCT), and lymphocytes (LY). As shown in FIGS. 34 and 35, no obvious changes were detected in any hematological parameter across groups, indicating negligible side effects of the CTCE-p53 NPs and their combination with aPD1. We also examined the major organs by H&E staining. Histological analyses revealed no obvious abnormality and no differences in the main organs among the treatment groups (FIGS. 36 and 37), further demonstrating the in vivo safety of the combination treatment.

### Example 10: Further Directions

The last decade has witnessed a tremendous shift in cancer treatment toward immunotherapy with ICBs, significantly extending the survival of cancer patients, including those with HCC. However, benefits may be seen in only a fraction of patients. Combinations of ICB therapy with other therapy modalities (e.g., chemotherapy, radiotherapy, and targeted therapy) are being actively explored for their ability to activate anti-tumor immune response and/or alter the immunosuppressive TME. These strategies are designed to increase the recruitment of activated effector T cells in ‘immunologically cold’ tumors that lack T cells and do not respond to ICB-based therapy.

The tumor suppressor p53 is one of the most frequently mutated genes in a wide range of cancers and is strongly associated with tumorigenesis, tumor progression, treatment resistance, and adverse prognosis. Compelling evidence suggests that p53 dysfunction can lead to immunosuppression and immune evasion. Restoration of p53 function thus may offer the opportunity to reverse immunosuppression of the TME and improve the anti-tumor efficacy of ICB therapy. Current efforts towards p53 reactivation include small molecules and DNA therapies,³¹⁻³⁷ which may be associated with certain drawbacks,³⁸⁻³⁹ highlighting the need for new therapeutic strategies to restore p53 functions.

The use of synthetic mRNA has attracted tremendous attention, as exemplified by the recent clinical approval of COVID-19 mRNA nano-vaccines and the clinical trials of a number of mRNA nanotherapeutics for diverse diseases including cancer.^{28,40-43} As a compelling alternative to DNA, mRNA requires only cytosolic delivery for translation, thus largely avoiding host genome integration and eliciting faster and more predictable protein expression. As described herein, we developed a CXCR4-targeted mRNA NP platform for effective p53 restoration and tested it in combination with aPD1 immunotherapy using p53-null murine HCC models. We extensively optimized the p53 mRNA NP platform by screening a series of ionizable lipid-like compounds and varying densities of CXCR4-targeting ligands for improving mRNA translation and HCC targeting in vivo. Our results demonstrate that the combination of CXCR4-targeted p53 mRNA NPs with aPD1 can lead to a potent antitumor effect in intrahepatic and ectopic models of HCC with p53 loss. The combination of p53 mRNA NPs and aPD1 effectively and globally reprogrammed the immune TME by promoting MHC-I expression and anti-tumor immunity, and decreasing the expression of immunosuppressive cytokines in HCC, irrespective of organ location. These findings suggest that p53 mRNA nanotherapy could enhance the efficacy of ICB therapy, substantially improving the treatment of p53-deficient HCC and potentially other p53-deficient cancers. Further studies can include those to understand the role of p53 in immune regulation, such as how the p53 status of cancer cells (e.g., p53 mutation) affects the immune TME and how the transfection of p53 mRNA NPs in immune cells (e.g., T cells, NK cells, and macrophages) affects their function in vivo. Compositions and methods that implement such understanding are encompassed by the present disclosure.

In addition, new combinatorial strategies between p53 targeting, ICB, with or without VEGF blockade may be required to increase durability of responses. If successfully translated, the mRNA nanotherapy-based p53 restoration strategy could be transformative and impactful in cancer immunotherapy. Compositions and methods including other agents (e.g., ICB, VEGF blockade, and the like) in any useful combinatorial manner are also encompassed by the present disclosure.

### Example 11: Transfection with Other Lipid-Like Compounds

The compositions and methods herein can be employed with any useful lipid-like compound. As discussed herein, compound having a generation 0 (Go) dendrimer can be used to deliver mRNA. Other dendrimers may be employed. In particular, we synthesized a series of ionizable lipid-like compounds (referred to as Gₘ-Cₙ compounds) based on different generations of poly(amidoamine) (PAMAM) dendrimers (G₀, G₁, G₂, G₃, and G₄ dendrimers) and epoxide chemistry to introduce different alkyl groups (substituted alkyl groups having 8, 10, 12, 14, or 16 carbons, e.g., —CH₂CH(OH)Cₙ₋₂H₂ₙ₋₃, in which n is 8, 10, 12, 14, or 16).

An optimized and robust self-assembly technique was employed to prepare mRNA-encapsulated polymer-lipid hybrid NPs having such Gₘ-Cₙ lipid-like compounds. Briefly, Gₘ-Cₙ and PLGA were dissolved separately in anhydrous DMF to form a homogeneous solution at concentrations of 2.5 mg/ml and 5 mg/ml, respectively. DSPE-MPEG was dissolved in DNase/RNase-free HyPure water at the concentration of 1 mg/mL. Citrate buffer with pH 3.0-3.5 was first added to 80 g of Gₘ-Cₙ(in 32 μl of DMF), then 16 μg of p53 mRNA (in 16 μl of citrate buffer) was added, mixed gently (at a Gₘ-Cₙ/mRNA weight ratio of 5), and allowed to stay at room temperature for 15 min to ensure the sufficient electrostatic complexation. Afterwards, 250 g of PLGA polymer (in 50 μl of DMF) was added to the mixture and gently mixed. The final mixture was added dropwise to 10 ml of DNase/RNase-free HyPure water having 1 mg of hybrid lipid-PEGs under uniform magnetic stirring (1000 rpm) for 30 min. An ultrafiltration device (EMD Millipore, MWCO 100 kDa) was used to remove the organic solvent and free compounds from the NP dispersion via centrifugation at 4° C. After washing three times with DNase/RNase-free HyPure water, the mRNA NPs were collected and finally concentrated in pH 7.4 PBS buffer. The NPs were used fresh or stored at −80° C. for further use.

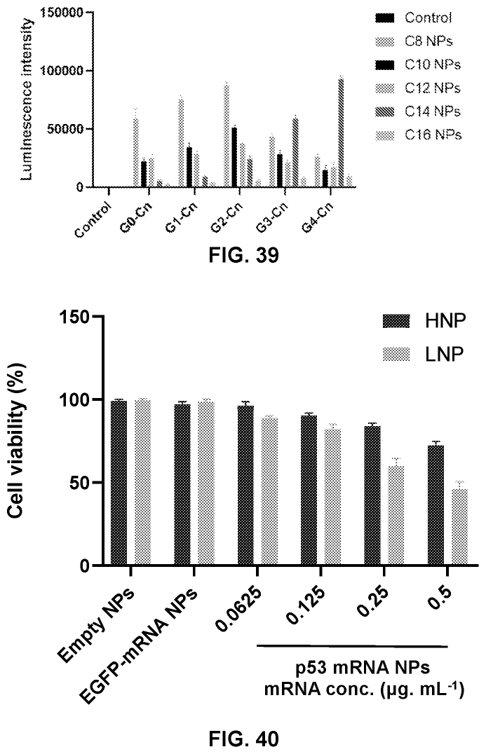

We screened the mRNA hybrid NPs for mRNA translation efficiency using a model luciferase-mRNA (Luc-mRNA). The chemical structures of these Gₘ-Cₙ were characterized by ¹H-NMR spectra. This non-limiting series of mRNA hybrid NPs was composed of PLGA polymer, Gₘ-Cₙ, and DSPE-PEG and showed an average size ˜95-120 nm with nearly neutral surface charge. From screening studies, we identified structure-activity relationships (FIG. 39). With respect to tail length, enhanced translation was observed in lipid-like compounds having tails that were 8 carbons in length or 14 carbons in length (FIG. 39). Of note, under these tested conditions, G₀-C₈, G₁-C₈, G₂-C₈, and G₄-C₁₄ showed the most effective mRNA translation. Based on this screening study, most of the Gₘ-Cₙ compounds could facilitate high luciferase mRNA transfection. Our results suggest that an optimized combination of amine group for Gₘ and tail length for Cₙ could impart desired mRNA delivery, and the present disclosure encompasses such combinations that can be present within the Gₘ-Cₙ compounds.

### Example 12: Effectiveness of Lipid Nanoparticles (LNP) and Hybrid Polymer-Lipid Nanoparticles (HNP)

The compositions herein can include any useful combination of mRNA and lipid-liked compounds that form particles (e.g., nanoparticles). In certain instances, the particle can be a hybrid polymer-lipid nanoparticle (HNP), which can include a polymer core and an outer layer including one or more lipids. In other instances, the particle can be a lipid nanoparticle (LNP), in which a polymer is not employed. Instead, the LNP can include the mRNA and lipid-like compound within a core of the particle, and the outer layer can include one or more lipids. For either the HNP or LNP, the outer layer can include any useful lipid (e.g., pegylated lipids, non-pegylated lipids, phospholipids, cholesterol, and/or other lipids described herein, as well as combinations thereof).

We generated a non-limiting LNP formulation having cholesterol, DSPC, ionizable lipid, and lipid-PEG, as well as an HNP formulation having a polymer. The LNPs included four components: an ionizable cationic lipid (e.g., DLin-MC3-DMA), a lipid-PEG (e.g., DMG-PEG), a phospholipid (e.g., DSPC), and cholesterol. The HNPs included another ionizable lipid-like compound (e.g., G₀-C₈), a biocompatible polymer (e.g., poly(lactic-co-glycolic acid) (PLGA)), and an outer layer including lipid-poly(ethylene glycol) (pegylated lipid).

Such formulations were tested for transfection efficiency, and p53 mRNA LNPs treatment can also led to cell death (FIG. 40). Accordingly, LNPs can be considered as an alternative mRNA formulation. Other lipid-containing nanoparticle formulations are also encompassed by the present disclosure (e.g., formulations including lipid nanoparticles, hybrid polymer-lipid nanoparticles, solid lipid nanoparticles, liposomes such as bilayer or multilayer liposomes, micelles, and the like, as well as combinations thereof).

### Example 13: Combination of p53 mRNA NPs with Radiotherapy

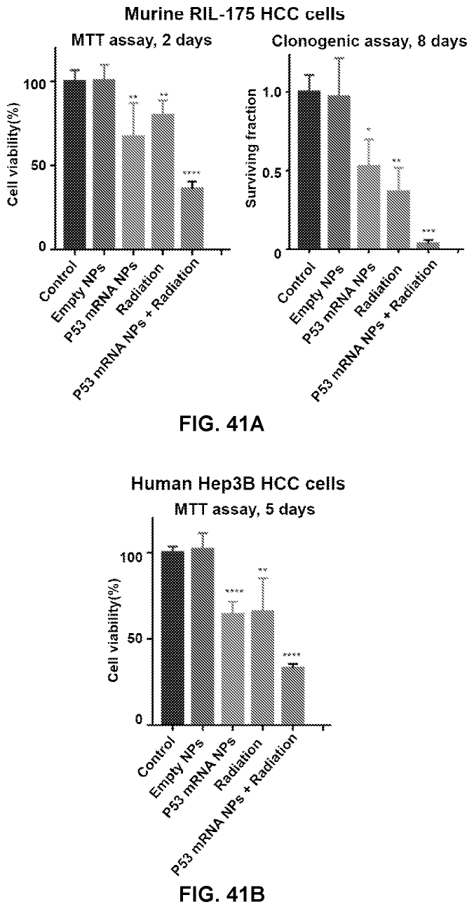

The compositions herein can be combined with one or more therapies. For example, such therapies can include any combination therapy described herein, including radiotherapy. Further experiments were conducted to determine the feasibility of combining NP-based treatment with radiotherapy. In particular, restoring p53 expression using hybrid nanoparticles (HNPs) sensitized human and murine HCC cells to irradiation in vitro, both in terms of acute cell killing (day 2) and clonogenic survival (day 8) (FIG. 41A-41B).

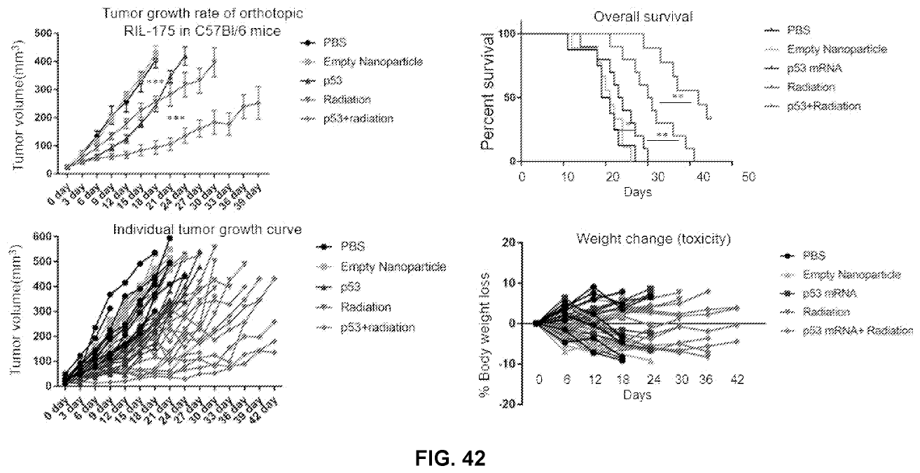

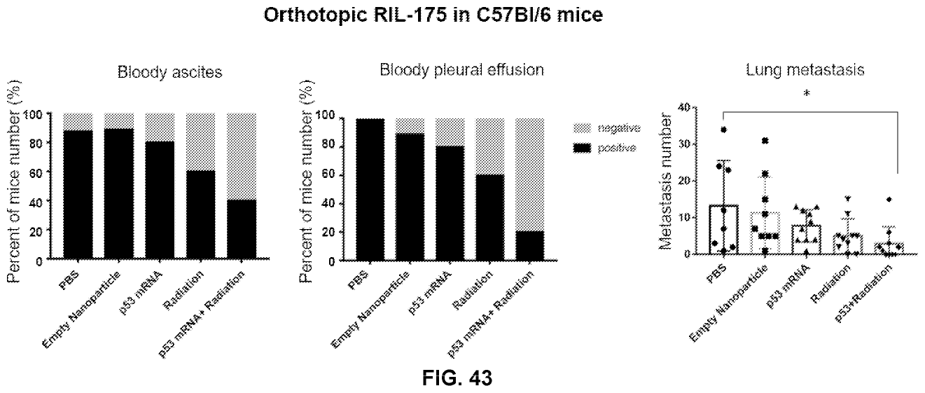

In vivo experiments were also conducted. As seen in FIG. 42, restoring p53 expression using NPs sensitized murine HCC tumors to irradiation in vivo, both in terms of tumor growth delay and overall survival, without overt toxicity (weight loss). Furthermore restoring p53 expression using NPs combined with radiotherapy reduced the morbidity of murine HCC tumors, both in terms of bloody ascites and pleural effusion formation, and decreased the incidence of lung metastases (FIG. 43).

Whilst the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references (including those listed above), scientific articles, patent publications, and any other documents cited herein are hereby incorporated by reference for the substance of their disclosure.

### REFERENCES

1. Kastenhuber E R & Lowe S W. Putting p53 in context. Cell 170, 1062-1078 (2017).
2. Guo G et al. Local activation of p53 in the tumor microenvironment overcomes immune suppression and enhances antitumor immunity. Cancer Res. 77, 2292 (2017).
3. Cui Y & Guo G. Immunomodulatory function of the tumor suppressor p53 in host immune response and the tumor microenvironment. Int. J. Mol. Sci. 17, 1942 (2016).
4. Lujambio A et al. Non-cell-autonomous tumor suppression by p53. Cell 153, 449-460 (2013).
5. Blagih J et al. p53, cancer and the immune response. J. Cell Sci. 133, jcs237453 (2020).
6. Bezzi M et al. Diverse genetic-driven immune landscapes dictate tumor progression through distinct mechanisms. Nat. Med. 24, 165-175 (2018).
7. Rodier F et al. Persistent DNA damage signalling triggers senescence-associated inflammatory cytokine secretion. Nat. Cell Biol. 11, 973-979 (2009).
8. Pathak S et al. Radiation and SN38 treatments modulate the expression of microRNAs, cytokines and chemokines in colon cancer cells in a p53-directed manner. Oncotarget 6, 44758-44780 (2015).
9. Xue W et al. Senescence and tumour clearance is triggered by p53 restoration in murine liver carcinomas. Nature 445, 656-660 (2007).
10. Tiwari N et al. p53- and PAI-1-mediated induction of C—X-C chemokines and CXCR2: importance in pulmonary inflammation due to cigarette smoke exposure. Am. J. Physiol. Lung Cell. Mol. Physiol. 310, L496-L506 (2016).
11. Owens D M. p53, chemokines, and squamous cell carcinoma. J. Clin. Invest. 117, 1752-1755 (2007).
12. Taura M et al. p53 regulates Toll-like receptor 3 expression and function in human epithelial cell lines. Mol. Cell. Biol. 28, 6557-6567 (2008).
13. Shatz M et al. p53 amplifies Toll-like receptor 5 response in human primary and cancer cells through interaction with multiple signal transduction pathways. Oncotarget 6, 16963-16980 (2015).
14. Sharma M D et al. Activation of p53 in immature myeloid precursor cells controls differentiation into Ly6c(+)CD103(+) monocytic antigen-presenting cells in tumors. Immunity 48, 91-106.e106 (2018).
15. Textor S et al. Human NK cells are alerted to induction of p53 in cancer cells by upregulation of the NKG2D ligands ULBP1 and ULBP2. Cancer Res. 71, 5998-6009 (2011).
16. Kim S S et al. A tumor-targeting nanomedicine carrying the p53 gene crosses the blood-brain barrier and enhances anti-PD-I immunotherapy in mouse models of glioblastoma. Int. J. Cancer 145, 2535-2546 (2019).
17. Li L et al. A unique role for p53 in the regulation of M2 macrophage polarization. Cell Death Differ. 22, 1081-1093 (2015).
18. Guo G et al. Local activation of p53 in the tumor microenvironment overcomes Immune Suppression and Enhances Antitumor Immunity. Cancer Res. 77, 2292-2305 (2017).
19. Tesniere A et al. Molecular characteristics of immunogenic cancer cell death. Cell Death Differ. 15, 3-12 (2008).
20. Levine A J et al. The p53 tumour suppressor gene. Nature 351, 453-456 (1991).
21. Petitjean A et al. TP53 mutations in human cancers: functional selection and impact on cancer prognosis and outcomes. Oncogene 26, 2157-2165 (2007).
22. Vogelstein B et al. Surfing the p53 network. Nature 408, 307-310 (2000).
23. Shigeta K et al. Dual programmed death receptor-1 and vascular endothelial growth factor receptor-2 blockade promotes vascular normalization and enhances antitumor immune responses in hepatocellular carcinoma. Hepatology 71, 1247-1261 (2020).
24. Shigeta K et al. Regorafenib combined with PD1 blockade increases CD8 T-cell infiltration by inducing CXCL10 expression in hepatocellular carcinoma. J.
Immunother. Cancer 8, e001435 (2020).
25. Finn R S et al. Atezolizumab plus bevacizumab in unresectable hepatocellular carcinoma. New Engl. J. Med. 382, 1894-1905 (2020).
26. Ren Z et al. Sintilimab plus a bevacizumab biosimilar (IB1I305) versus sorafenib in unresectable hepatocellular carcinoma (ORIENT-32): a randomised, open-label, phase 2-3 study. Lancet Oncol. 22, 977-990 (2021).
27. Zhu X et al. Long-circulating siRNA nanoparticles for validating Prohibitinl-targeted non-small cell lung cancer treatment. Proc. Natl Acad. Sci. USA 112, 7779 (2015).
28. Islam M A et al. Restoration of tumour-growth suppression in vivo via systemic nanoparticle-mediated delivery of PTEN mRNA. Nat. Biomed. Eng. 2, 850-864 (2018).
29. Chen Y et al. CXCR4 inhibition in tumor microenvironment facilitates anti-programmed death receptor-I immunotherapy in sorafenib-treated hepatocellular carcinoma in mice. Hepatology 61, 1591-1602 (2015).
30. Chen Y et al. Overcoming sorafenib evasion in hepatocellular carcinoma using CXCR4-targeted nanoparticles to co-deliver MEK-inhibitors. Sci. Rep. 7, 44123 (2017).
31. Bykov VJN et al. Reactivation of mutant p53 and induction of apoptosis in human tumor cells by maleimide analogs. J. Biol. Chem. 280, 30384-30391 (2005).
32. Muller PAJ & Vousden K H. Mutant p53 in cancer: new functions and therapeutic opportunities. Cancer Cell 25, 304-317 (2014).
33. Bykov VJN et al. Restoration of the tumor suppressor function to mutant p53 by a low-molecular-weight compound. Nat. Med. 8, 282-288 (2002).
34. Vassilev L T et al. In vivo activation of the p53 pathway by small-molecule antagonists of MDM2. Science 303, 844 (2004).
35. van Beusechem V W et al. Conditionally replicative adenovirus expressing p53 exhibits enhanced oncolytic potency. Cancer Res. 62, 6165 (2002).
36. Guan Y S et al. p53 gene (Gendicine) and embolisation overcame recurrent hepatocellular carcinoma. Gut 54, 1318 (2005).
37. Pirollo K F et al. Safety and efficacy in advanced solid tumors of a targeted nanocomplex carrying the p53 gene used in combination with docetaxel: a phase lb study. Mol. Ther. 24, 1697-1706 (2016).
38. Cheok C F et al. Translating p53 into the clinic. Nat. Rev. Clin. Oncol. 8, 25-37 (2011).
39. Kapoor R et al. Challenges in the gene therapy commercial ecosystem. Nat. Biotechnol. 35, 813-815 (2017).
40. Hou X et al. Vitamin lipid nanoparticles enable adoptive macrophage transfer for the treatment of multidrug-resistant bacterial sepsis. Nat. Nanotechnol. 15, 41-46 (2020).
41. Li B et al. Engineering CRISPR-Cpf1 crRNAs and mRNAs to maximize genome editing efficiency. Nat. Biomed. Eng. 1, 0066 (2017).
42. Miao L et al. Delivery of mRNA vaccines with heterocyclic lipids increases anti-tumor efficacy by STING-mediated immune cell activation. Nat. Biotechnol. 37, 1174-1185 (2019).
43. Kong N et al. Synthetic mRNA nanoparticle-mediated restoration of p53 tumor suppressor sensitizes p53-deficient cancers to mTOR inhibition. Sci. Transl. Med. 11, eaawl565 (2019).
44. Xiao Y et al. Combining p53 mRNA nanotherapy with immune checkpoint blockade reprograms the immune microenvironment for effective cancer therapy. Nat. Commun. 13, 758 (2022).

1. A method of treating a cancer, the method comprising administering a therapeutically effective amount of an mRNA encoding tumor suppressor protein p53 with a combination therapy to a subject in need thereof.

2. The method of claim 1, wherein the combination therapy comprises immunotherapy, anti-angiogenesis therapy, radiotherapy, or a combination thereof.

3. The method of claim 2, wherein the immunotherapy comprises administering a therapeutically effective amount of at least one immune checkpoint inhibitor to the subject.

4. The method of claim 3, wherein the at least one immune checkpoint inhibitor is an anti-PD1 antibody, an anti-PD-L1 antibody, an anti-CTLA-4 antibody, an anti-TIM3 antibody, an anti-LAG3 antibody, an anti-CD137 antibody, or an anti-CD40 antibody.

5. The method of claim 2, wherein the anti-angiogenesis therapy comprises administering a therapeutically effective amount of at least one angiogenesis inhibitor to the subject.

6. The method of claim 5, wherein the at least one angiogenesis inhibitor is an anti-VEGF antibody, an anti-VEGF receptor antibody, a VEGF receptor kinase inhibitor, an anti-FGF antibody, an anti-FGF receptor antibody, an FGF receptor kinase inhibitor, an anti-PDGF antibody, an anti-PDGF receptor antibody, a PDGF receptor kinase inhibitor, an anti-EGF antibody, an anti-EGF receptor antibody, or an EGF receptor kinase inhibitor.

7. The method of claim 2, wherein the radiotherapy comprises administering a therapeutically effective amount of irradiation to the subject.

8. The method of claim 7, wherein the irradiation comprises x-rays, gamma rays, electron beam radiation, proton beam radiation, or ionizing particles.

9. The method of claim 1, wherein the cancer is associated with loss of p53 expression or activity.

10. The method of claim 1, wherein the cancer is selected from the group consisting of liver cancer, lung cancer, prostate cancer, breast cancer, glioblastoma, melanoma, pancreatic cancer, colorectal cancer, and leukemia.

11. The method of claim 1, wherein the p53-encoding mRNA is within a delivery vehicle capable of providing release of the p53-encoding mRNA in a cancer cell.

12. The method of claim 11, wherein the delivery vehicle is a particle comprising the p53-encoding mRNA and a complexing agent within a core.

13. The method of claim 12, wherein the particle further comprises an outer layer comprising at least one amphiphilic material disposed around the core.

14. The method of claim 13, wherein the amphiphilic material is selected from a group consisting of lecithin, a phospholipid, and a pegylated lipid.

15. The method of claim 14, wherein the phospholipid is selected from a group consisting of phosphatidic acid, phosphatidylcholine, phosphatidylethanolamine, phosphatidylglycerol, phosphatidylserine, phosphatidylinositol, lysophosphatidyl, cardiolipin, and β-acyl-y-alkyl phospholipid.

16. The method of claim 14, wherein the pegylated lipid is selected from a group consisting of ceramide-polyethylene glycol (PEG) and 1,2-distearoyl-sn-glycero-3-phosphoethanolamine (DSPE)-terminated PEG.

17. The method of claim 12, wherein the complexing agent is selected from a group consisting of a cationic lipid, an ionizable lipid-like compound, and an ionizable lipid.

18. The method of claim 17, wherein the ionizable lipid-like compound is Gₘ-Cₙ, wherein m≥0 and n<20; and wherein the ionizable lipid is DLin-MC3-DMA, SM-102, or ALC-0315.

19. The method of claim 12, wherein the core further comprises a water-insoluble polymer.

20. The method of claim 19, wherein the water-insoluble polymer is a polyester selected from a group consisting of poly(lactic-co-glycolic acid) (PLGA), poly(lactic acid) (PLA), and poly(glycolic acid) (PGA); or a copolymer of polyethylene glycol (PEG) and a polyester selected from a group consisting of PLGA, PLA, PGA.

21. A composition comprising:
a core, wherein the core comprises:
a tumor suppressor-encoding mRNA;
an ionizable lipid-like compound of Gₘ-Cₙ, wherein m≥0 and n<20; and
an optional water-insoluble polymer; and
an outer layer surrounding the core, wherein the outer layer comprises:
a targeting ligand for a chemokine receptor; and
a pegylated lipid.

22. The composition of claim 21, wherein the targeting ligand comprises: a targeting moiety configured to bind to the chemokine receptor, a pegylated lipid configured to form a portion of the outer layer, and a linker disposed between the targeting moiety and the pegylated lipid.

23. The composition of claim 21, wherein the tumor suppressor-encoding mRNA comprises an mRNA encoding a p53 protein, and/or wherein the targeting ligand comprises a CXCR4-targeting ligand.

24. The composition of claim 23, wherein the CXCR4-targeting ligand comprises a CXCR4-targeting moiety bound to a lipid and an optional linker disposed between the CXCR4-targeting moiety and the lipid.

25. The composition of claim 24, wherein the CXCR4-targeting moiety comprises a sequence having at least 80% sequence identity to KGVSLSYRCRYSLSVGK (SEQ ID NO: 1) or a fragment thereof.

26. The composition of claim 23, wherein a density of the CXCR4-targeting ligand is between about 3% to about 10%.

27. The composition of claim 21, wherein the tumor suppressor-encoding mRNA comprises an mRNA encoding a p53 protein, and/or wherein the targeting ligand comprises a GPC3-targeting ligand.

28. The composition of claim 21, wherein the outer layer further comprises a pegylated lipid, a lipid, or a combination thereof.

29. The composition of claim 21, wherein m is an integer from 0 to 5, and wherein n is an integer from 6 to 18.

30. The composition of claim 29, wherein the ionizable lipid-like compound comprises G₀-C₈, G₀-C₁₀, G₀-C₁₂, G₁-C₈, G₁-C₁₀, G₁-C₁₂, G₁-C₁₄, G₂-C₈, G₂-C₁₀, G₂-C₁₂, G₂-C₁₄, G₃-C₈, G₃-C₁₀, G₃-C₁₂, G₃-C₁₄, G₄-C₈, G₄-C₁₀, G₄-C₁₂, or G₄-C₁₄.

31. The composition of claim 21, wherein a weight ratio of the ionizable lipid-like compound to the mRNA is from about 1:1 to about 40:1 (wt:wt).

32. The composition of claim 21, wherein the core and the outer layer forms a nanoparticle, and wherein the nanoparticle has an average size from about 80 nm to about 150 nm.

33. The composition of claim 21, wherein the ionizable lipid-like compound is not G₀-C₁₄.

34. A composition comprising:
a core, wherein the core comprises:
a nucleic acid;
a lipid-like compound of Gₘ-Cₙ, wherein (i) m=0 and n<14; or (ii) m>0 and n<20; and
an optional water-insoluble polymer; and
an outer layer surrounding the core, wherein the outer layer comprises:
a lipid or a pegylated lipid; and
an optional targeting ligand.

35. The composition of claim 34, wherein the targeting ligand comprises a protein, a peptide, an aptamer, a nucleic acid, a monosaccharide, a polysaccharide, a carbohydrate, a vitamin, or a small molecule.

36. The composition of claim 34, wherein m is 0; and wherein n is an integer from about 6 to about 12.

37. The composition of claim 34, wherein m is 1; and wherein n is an integer from about 6 to about 12.

38. The composition of claim 34, wherein m is 2, 3, or 4; and wherein n is an integer from about 6 to about 14.

39. The composition of claim 34, wherein m is 0, 1, 2, or 3; and wherein n is about 8.

40. The composition of claim 34, wherein m is 3 or 4; and wherein n is about 14.

41. A formulation comprising a therapeutically effective amount of a composition of any one of claims 21-40 and a pharmaceutically acceptable excipient.

42. The formulation of claim 41, wherein the formulation is formulated for injection, implantation, and the like.

43. A method of treating cancer, the method comprising:
administering a therapeutically effective amount of a composition of any one of claims 21-40 or a formulation of any one of claims 41-42 to a subject in need thereof.

44. The method of claim 43, wherein the cancer is a p53-deficient cancer, a p53-deficient cancer primary liver cancer, or liver metastases from a p53-deficient cancer.

45. The method of claim 44, wherein the p53-deficient cancer is primary colon cancer, primary lung cancer, primary liver cancer, primary hepatocellular carcinoma, primary cholangiocarcinoma, primary pancreatic cancer, metastatic colon cancer, metastatic lung cancer, metastatic liver cancer, or metastatic pancreatic cancer.

46. The method of claim 43, further comprising:
administering a therapeutically effective amount of at least one immune checkpoint inhibitor at a timepoint before, after, or during said administering the composition.

47. The method of claim 46, wherein the at least one immune checkpoint inhibitor comprises an anti-PD-1 (aPD1) antibody, an anti-PD-L1 (aPD-L1) antibody, an anti-CTLA-4 antibody, an anti-TIM3 antibody, an anti-LAG3 antibody, an anti-CD137 antibody, or an anti-CD40 antibody.

48. The method of claim 22, further comprising:
administering a therapeutically effective amount of at least one angiogenesis inhibitor at a timepoint before, after, or during said administering the composition.

49. The method of claim 22, further comprising:
administering a therapeutically effective amount of irradiation at a timepoint before, after, or during said administering the composition.

50. A method of modulating an interaction between a tumor and an immune cell, the method comprising:
administering a therapeutically effective amount of a composition of any one of claims 21-40 or a formulation of any one of claims 41-42 to a subject in need thereof.

51. The method of claim 50, wherein the immune cell comprises an NK cell, a T cell, and/or a tumor-associated macrophage (TAM).

52. The method of claim 50, further comprising:
administering a therapeutically effective amount of at least one immune checkpoint inhibitor at a timepoint before, after, or during said administering the composition.

53. The method of claim 52, wherein the at least one immune checkpoint inhibitor comprises an anti-PD-1 (aPDI) antibody, an anti-PD-L1 (aPD-L1) antibody, an anti-CTLA-4 antibody, an anti-TIM3 antibody, an anti-LAG3 antibody, an anti-CD137 antibody, or an anti-CD40 antibody.

54. The method of claim 50, further comprising:
administering a therapeutically effective amount of at least one VEGF inhibitor at a timepoint before, after, or during said administering the composition.

55. The method of claim 50, further comprising:
administering a therapeutically effective amount of irradiation at a timepoint before, after, or during said administering the composition.

56. A method of making a composition, the method comprising:
complexing an mRNA with a lipid-like compound of Gₘ-Cₙ in an acidic environment, wherein m≥0 and n<20, in the presence of an optional water-insoluble polymer, thereby forming a core; and
surrounding the core with an outer layer comprising a lipid, a pegylated lipid, or a target ligand, thereby providing the composition.

57. The method of claim 56, wherein the acidic environment comprises a pH from about 4 to about 2.

58. The method of claim 56, wherein the acidic environment comprises an acidic buffered solution.

59. The method of claim 56, wherein said forming comprises the water-insoluble polymer in an organic solvent.

60. The method of claim 56, wherein said surrounding comprises stirring at a rate of about 500 rpm to about 1500 rpm.

61. The method of claim 56, wherein the outer layer comprises the pegylated lipid and the target ligand.

62. The method of claim 56, wherein the method provides the composition of any one of claims 21-40.
